SEQUENCE LISTING (25 sequences)

SEQ ID NO: 1 - protein, 447 aa, homo sapiens
QVQLVQSGAEVKKPGASVKVSCKASGYTFTSYWMHWVRQAPGQGLEWIGNIYPGSGSTFYDEKFKGRATLTVDTSISTAYMELSRLRSDDTAVYYCTRRNYVTGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVGVSHEDPEVKFNWYVDGVEVHNAKTKPREEQYNSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 468 aa, homo sapiens
DIQLTQSPSFLSASVGDRVTITCKSSQSVLYSSNQKNYLAWYQQKPGKAPKLLIYWASTRESGVPSRFSGSGSGTEFTLTISSLQPEDFATYYCHQYLSSYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASQVQLVESGGGVVQPGRSLRLSCAASGFSLSDYGVHWVRQAPGKGLEWLGVIWAGGGTNYNSALMSRKTISKDNSKNTVYLQMNSLRAEDTAVYYCARDKGYSYYYSMDYWGQGTTVTVSSGGGGSGGGGSGGGGSDIQMTQSPSTLSASVGDRVTIICRASESVEYYVTSLMQWYQQKPGKAPKLLIFAASNVESGVPSRFSGSGSGTEFTLTISSLQPDDFATYYCQQSRKVPYTFGQGTKLEIKR

SEQ ID NO: 3 - protein, 483 aa, homo sapiens
DIQLTQSPSFLSASVGDRVTITCKSSQSVLYSSNQKNYLAWYQQKPGKAPKLLIYWASTRESGVPSRFSGSGSGTEFTLTISSLQPEDFATYYCHQYLSSYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASGGGGSGGGGSGGGGSQVQLVESGGGVVQPGRSLRLSCAASGFSLSDYGVHWVRQAPGKGLEWLGVIWAGGGTNYNSALMSRKTISKDNSKNTVYLQMNSLRAEDTAVYYCARDKGYSYYYSMDYWGQGTTVTVSSGGGGSGGGGSGGGGSDIQMTQSPSTLSASVGDRVTIICRASESVEYYVTSLMQWYQQKPGKAPKLLIFAASNVESGVPSRFSGSGSGTEFTLTISSLQPDDFATYYCQQSRKVPYTFGQGTKLEIKR

SEQ ID NO: 4 - protein, 696 aa, homo sapiens
QVQLVQSGAEVKKPGASVKVSCKASGYTFTSYWMHWVRQAPGQGLEWIGNIYPGSGSTFYDEKFKGRATLTVDTSISTAYMELSRLRSDDTAVYYCTRRNYVTGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVGVSHEDPEVKFNWYVDGVEVHNAKTKPREEQYNSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKASQVQLVESGGGVVQPGRSLRLSCAASGFSLSDYGVHWVRQAPGKGLEWLGVIWAGGGTNYNSALMSRKTISKDNSKNTVYLQMNSLRAEDTAVYYCARDKGYSYYYSMDYWGQGTTVTVSSGGGGSGGGGSGGGGSDIQMTQSPSTLSASVGDRVTIICRASESVEYYVTSLMQWYQQKPGKAPKLLIFAASNVESGVPSRFSGSGSGTEFTLTISSLQPDDFATYYCQQSRKVPYTFGQGTKLEIKR

SEQ ID NO: 5 - protein, 711 aa, homo sapiens
QVQLVQSGAEVKKPGASVKVSCKASGYTFTSYWMHWVRQAPGQGLEWIGNIYPGSGSTFYDEKFKGRATLTVDTSISTAYMELSRLRSDDTAVYYCTRRNYVTGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVGVSHEDPEVKFNWYVDGVEVHNAKTKPREEQYNSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKASGGGGSGGGGSGGGGSQVQLVESGGGVVQPGRSLRLSCAASGFSLSDYGVHWVRQAPGKGLEWLGVIWAGGGTNYNSALMSRKTISKDNSKNTVYLQMNSLRAEDTAVYYCARDKGYSYYYSMDYWGQGTTVTVSSGGGGSGGGGSGGGGSDIQMTQSPSTLSASVGDRVTIICRASESVEYYVTSLMQWYQQKPGKAPKLLIFAASNVESGVPSRFSGSGSGTEFTLTISSLQPDDFATYYCQQSRKVPYTFGQGTKLEIKR

SEQ ID NO: 6 - protein, 219 aa, homo sapiens
DIQLTQSPSFLSASVGDRVTITCKSSQSVLYSSNQKNYLAWYQQKPGKAPKLLIYWASTRESGVPSRFSGSGSGTEFTLTISSLQPEDFATYYCHQYLSSYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 710 aa, homo sapiens
QVQLVESGGGVVQPGRSLRLSCAASGFSLSDYGVHWVRQAPGKGLEWLGVIWAGGGTNYNSALMSRKTISKDNSKNTVYLQMNSLRAEDTAVYYCARDKGYSYYYSMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVGVSHEDPEVKFNWYVDGVEVHNAKTKPREEQYNSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKASGGGGSGGGGSGGGGSQVQLVQSGAEVKKPGASVKVSCKASGYTFTSYWHWVRQAPGQGLEWIGNIYPGSGSTFYDEKFKGRATLTVDTSISTAYMELSRLRSDDTAVYYCTRRNYVTGFDYWGQGTTVTVSSGGGGSGGGGSGGGGSDIQLTQSPSFLSASVGDRVTITCKSSQSVLYSSNQKNYLAWYQQKPGKAPKLLIYWASTRESGVPSRFSGSGSGTEFTLTISSLQPEDFATYYCHQYLSSYTFGQGTKLEIK

SEQ ID NO: 8 - protein, 711 aa, homo sapiens
QVQLVESGGGVVQPGRSLRLSCAASGFSLSDYGVHWVRQAPGKGLEWLGVIWAGGGTNYNSALMSRKTISKDNSKNTVYLQMNSLRAEDTAVYYCARDKGYSYYYSMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVGVSHEDPEVKFNWYVDGVEVHNAKTKPREEQYNSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKASGGGGSGGGGSGGGGSDIQLTQSPSFLSASVGDRVTITCKSSQSVLYSSNQKNYLAWYQQKPGKAPKLLIYWASTRESGVPSRFSGSGSGTEFTLTISSLQPEDFATYYCHQYLSSYTFGQGTKLEIKGGGGSGGGGSGGGGSQVQLVQSGAEVKKPGASVKVSCKASGYTFTSYWMHWVRQAPGQGLEWIGNIYPGSGSTFYDEKFKGRATLTVDTSISTAYMELSRLRSDDTAVYYCTRRNYVTGFDYWGQGTTVTVSS

SEQ ID NO: 9 - protein, 219 aa, homo sapiens
DIQMTQSPSTLSASVGDRVTIICRASESVEYYVTSLMQWYQQKPGKAPKLLIFAASNVESGVPSRFSGSGSGTEFTLTISSLQPDDFATYYCQQSRKVPYTFGQGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 693 aa, homo sapiens
EVQLEQSGPELVKPGTSVKISCKTSGYTFTEYTMHWVKQSHGKSLEWIGGINPNNGGTTYNQIFKNKATLTVDKSSSTAYMELRSLTSEDSAVYYCARRGYHVSSWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVGVSHEDPEVKFNWYVDGVEVHNAKTKPREEQYNSTYRVVSVLTVLHQDWLNGKEYKCKVSNKQLPSPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGQVQLVQSGAEVKKPGASVKVSCKASGYTFTSYVMHWVRQAPGQGLEWIGYINPYNDVTKYNEKFKGRVTMTRDTSTSTVYMELSSLRSEDTAVYYCARGSYYDYDGFVYWGQGTLVTVSSGGGGSGGGGSGGGGSEIVLTQSPATLSLSPGERATLSCSATSSVSYMHWYQQKPGLAPRRWIYDTSKLASGIPDRFSGSGSGTDFTLTISRLEPEDFAVYYCQQWSSNPLTFGAGTKLELK

SEQ ID NO: 11 - protein, 213 aa, homo sapiens
DIVLTQSTAIMSASPGEKVTMTCSASSSVSYMHWYQQKSGTSPKRWIYDTSKLASGVPARFSGSGSGTSYSLTISSMETEDSATYYCLQWHSNPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 448 aa, artificial
  Heavy chain_huEng (Kro-22.2) Fc silenced optimized
QVQLVQSGAEVKKPGASVKVSCKASGYTFTSYWMHWVRQAPGQGLEWIGNIYPGSGSTFYDEKFKGRATLTVDTSISTAYMELSRLRSDDTAVYYCTRRNYVTGFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 488 aa, artificial
  LC+L (Light chain)_huEng (Kro-22.6)_kappa_ CD28(hu9.3.8.V1) optimized
DIQLTQSPSFLSASVGDRVTITCKSSQSVLYSSNQKNYLAWYQQKPGKAPKLLIYWASTRESGVPSRFSGSGSGTEFTLTISSLQPEDFATYYCHQYLSSYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECASGGGGSGGGGSGGGGSGGSQVQLVQSGGGVVQPGQSLRLSCAASGFSLSDYGVHWVRQAPGKGLEWLGVIWAGGGTNYNSALQSRFTISKDNSKNTVYLQMNSLRAEDTAVYYCARDKGYSYYYSMDYWGQGTTVTVSSGGGGSGGGGSGGGGSGGSDIQMTQSPSTLSASVGDRVTITCRASESVEYYVTSLMQWYQQKPGKAPKLLIFAASNVESGVPSRFSGSGSGTEFTLTISSLQPDDFATYYCQQSRKVPYTFGQGTKLEIK

SEQ ID NO: 14 - protein, 5 aa, artificial
  Endoglin humanized AB Clone Kro22.2
SYWMH

SEQ ID NO: 15 - protein, 17 aa, artificial
  Endoglin humanized AB Clone Kro22.2
NIYPGSGSTFYDEKFKG

SEQ ID NO: 16 - protein, 9 aa, artificial
  Endoglin humanized AB Clone Kro22.2
RNYVTGFDY

SEQ ID NO: 17 - protein, 118 aa, artificial
  Endoglin humanized AB Clone Kro22.2
QVQLVQSGAEVKKPGASVKVSCKASGYTFTSYWMHWVRQAPGQGLEWIGNIYPGSGSTFYDEKFKGRATLTVDTSISTAYMELSRLRSDDTAVYYCTRRNYVTGFDYWGQGTTVTVSS

SEQ ID NO: 18 - protein, 17 aa, artificial
  Endoglin humanized AB Clone Kro22.6
KSSQSVLYSSNQKNYLA

SEQ ID NO: 19 - protein, 7 aa, artificial
  Endoglin humanized AB Clone Kro22.6
WASTRES

SEQ ID NO: 20 - protein, 8 aa, artificial
  Endoglin humanized AB Clone Kro22.6
HQYLSSYT

SEQ ID NO: 21 - protein, 112 aa, artificial
  Endoglin humanized AB Clone Kro22.6
DIQLTQSPSFLSASVGDRVTITCKSSQSVLYSSNQKNYLAWYQQKPGKAPKLLIYWASTRESGVPSRFSGSGSGTEFTLTISSLQPEDFATYYCHQYLSSYTFGQGTKLEIK

SEQ ID NO: 22 - DNA, 354 bp, artificial
  Endoglin humanized AB Clone Kro22.2
caggttcagctggttcagtctggcgccgaagtgaagaaacctggcgcctctgtgaaggtgtcctgcaaggccagcggctacacctttaccagctactggatgcactgggtccgacaggctccaggacaaggcctggaatggatcggcaacatctaccctggcagcggcagcaccttctacgacgagaagttcaagggcagagccacactgaccgtggacaccagcatcagcaccgcctacatggaactgagccggctgagatccgatgacaccgccgtgtactactgcaccagacggaattacgtgaccggcttcgactattggggccagggcacaaccgtgaccgttagctct

SEQ ID NO: 23 - DNA, 336 bp, artificial
  Endoglin humanized AB Clone Kro22.6
gacatccagctgactcagagccctagctttctgagcgccagcgtgggcgacagagtgaccatcacctgtaaaagcagccagagcgtgctgtactccagcaaccagaagaactacctggcctggtatcagcagaagcccggcaaggctcctaagctgctgatctactgggccagcaccagagaaagcggcgtgccaagcagattttctggcagcggctctggcaccgagttcaccctgaccatatctagcctgcagcctgaggacttcgccacctactactgccaccagtacctgagcagctacacctttggccagggcaccaagctggaaatcaag

SEQ ID NO: 24 - protein, 249 aa, artificial
  Optimized anti-CD28 scFv
QVQLVQSGGGVVQPGQSLRLSCAASGFSLSDYGVHWVRQAPGKGLEWLGVIWAGGGTNYNSALQSRFTISKDNSKNTVYLQMNSLRAEDTAVYYCARDKGYSYYYSMDYWGQGTTVTVSSGGGGSGGGGSGGGGSGGSDIQMTQSPSTLSASVGDRVTITCRASESVEYYVTSLMQWYQQKPGKAPKLLIFAASNVESGVPSRFSGSGSGTEFTLTISSLQPDDFATYYCQQSRKVPYTFGQGTKLEIK

SEQ ID NO: 25 - protein, 5 aa, homo sapiens
GGGGS

(19) United States
(12) Patent Application Publication
(10) Pub. No.: US 2023/0406961 A1
(43) Pub. Date: Dec. 21, 2023

(54) TARGET-CELL RESTRICTED, COSTIMULATORY, BISPECIFIC AND BIVALENT ANTI-CD28 ANTIBODIES

(71) Applicants: Deutsches Krebsforschungszentrum Stiftung des öffentlichen Rechts, Heidelberg (DE); Eberhard Karls Universität Tübingen, Tübingen (DE)

(72) Inventors: Helmut SALIH, Tübingen (DE); Gundram JUNG, Rottenburg (DE); Martin PFLÜGLER, Tübingen (DE); Latifa ZEKRI, Heidelberg (DE); Timo MANZ, Heidelberg (DE)

(73) Assignees: Deutsches Krebsforschungszentrum Stiftung des öffentlichen Rechts, Heidelberg (DE); Eberhard Karls Universität Tübingen, Tübingen (DE)

(21) Appl. No.: 18/035,082

(22) PCT Filed: Nov. 3, 2021
(86) PCT No.: PCT/EP2021/080564

§ 371 Date: May 2, 2023

(30) Foreign Application Priority Data
Nov. 3, 2020 (EP) 20205442.5

(51) Int. Cl. C07K 16/46; A61P 35/00

(52) U.S. Cl. CPC C07K 16/468; A61P 35/00; C07K 2317/31; C07K 2317/622

(57) ABSTRACT

The present invention provides a novel bispecific anti-CD28 antibody format which is bivalent and comprises two CD28 binding sites, and at least one target binding site. The bispecific anti CD28 antibody of the invention is surprisingly advantageous due to its costimulatory activity which is strictly target cell restricted. The bispecific CD28 antibody of the invention is provided for use in the treatment of diseases either alone or in combination with a further bispecific antibody inducing a CD3/T cell receptor signal.

### FIELD OF THE INVENTION

The present invention provides a novel bispecific anti-CD28 antibody format which is bivalent and comprises two CD28 binding sites, and at least one target binding site. The bispecific anti CD28 antibody of the invention is surprisingly advantageous due to its costimulatory activity which is strictly target cell restricted. The bispecific CD28 antibody of the invention is provided for use in the treatment of diseases either alone or in combination with a further bispecific antibody inducing a CD3/T cell receptor signal.

### DESCRIPTION

The activation of T cells is central for the initiation of an antigen specific immune response. In the past decades numerous T cell surface receptors have been identified that shape this process. Of central importance is the antigen-specific T cell receptor (TCR)/CD3 complex. Upon binding of antigenic peptides presented by MHC molecules by the TCR the signal via the CD3 part of the complex, also termed “signal one”, initiates T cell activation [1]. Importantly, for an effective and sustained response additional costimulatory signals (“second signals”) via receptors like CD28, 4-1BB or Ox40 are required [2,3]. In fact, in the absence of such signals an isolated TCR/CD3 stimulus might eventually result in T cell non-responsiveness or even—death, and thus costimulatory signals appear to be a central prerequisite for an effective T cell response [4,5]

The identification of the surface receptors involved in T cell activation is the basis of two functionally very similar strategies that aim to recruit T cells against tumor cells [6]:
CAR T cells are transfected with a chimeric receptor comprising an antibody moiety that binds to a tumor associated antigen (TAA) and the intracellular signaling motifs of the CD3 molecule [7].
Similarly, bispecific antibodies (bsAb) have been constructed that bind to TAM as well as to the CD3 molecule, designated here bsAbCD3 [8].

Both types of reagents are capable of redirecting T cells towards tumor cells independent of the antigen specificity of the T cells and have been suggested already in the late eighties [9-13]. Equipped with antibody moieties directed to CD19 they are nowadays firmly established for the treatment of B cell derived leukemias and lymphomas. However, both reagent types also share a major side effect: excessive generalized T cell activation resulting in an—often severe- and dose limiting cytokine release syndrome [14]. There is, however also a remarkable difference: The “first generation” CAR T cells contained only CD3-derived signaling motifs and were of limited therapeutic efficiency. Only when “second generation” CAR constructs were equipped with additional motifs derived from the costimulatory molecules CD28 and/or 4-1BB they were capable of exerting durable therapeutic effects [15]. Notably a combinatorial application of bispecific antibodies with TAAxCD3- and TAAxCD28 has been suggested already in the late eighties but was not developed to the stage of regulatory approval. This was due to the considerable technical effort required to realize such an approach but also to the negative impact of the so called Tegenero incident in 2006, when six healthy volunteers experienced a severe cytokine release syndrome during the infusion of a superagonistic CD28 antibody that required immediate intensive medical care for all participants [17]. This illustrated dramatically that the agonistic activity of T cell stimulating antibodies is to be target cell restricted, that is, dependent upon binding to the respective target antigen, to avoid untolerable side effects upon clinical application. In practice this may be achieved by (i) choosing a suitable, Fc-depleted antibody format to prevent T cell activation by FcR expressing cells rather than by target cells and (ii) by careful design of the affinity and/or valency of the stimulating antibody.

Recombinant antibody technology offers multiple formats for the construction of bi- and multi-functional antibody-based molecules [18]. The benchmark bsAb blinatumomab is an antibody with a CD19xCD3 specificity in the so-called bispecific T cell engager (BiTE)-format that consists of two single chain antibodies fused by a glycin serin linker. This format supports target cell restriction due to the lack of Fc parts and the univalency and rather low affinity of the CD3 binding part. However, BiTE molecules have a low molecular weight resulting in correspondingly low serum half lifes of approx. 1 hr. This requires cumbersome continuous infusion regimes. Full length IgG based formats that are Fc-attenuated by selected mutation in the CH2 domain offer the advantage of a considerably larger half-life but require careful design in particular if the T cell stimulating part is bivalent and directed to CD28 rather than CD3. This is because in general, CD3 antibodies in a bivalent form do not activate T cells [19]. They require immobilization, e.g. by binding to FcR expressing cells. In contrast CD28 antibodies may deliver efficient costimulation as soluble bivalent antibodies [20]. Thus, it appears that, in principle, it is difficult to achieve target cell restricted activity with TAAxCD28 antibodies that contain bivalent CD28 binding parts.

In this application the present invention describes a bispecific TAAxCD28 antibodies with bivalent CD28 binding parts that are entirely target cell restricted to avoid systemic T cell activation during clinical application of these reagents.

Even if target cell restriction is achieved specificity problems remain in as much as the selected target antigens are —in almost all cases—expressed on normal, healthy tissue. This results in undesirable “off-tumor on target” activation of T cells. This problem is exemplified by the well-known side effects of blinatumomab, a BsAbCD3 that targets CD19, an antigen that is expressed on normal B cells and on brain mural cells [21]. This results in systemic T cell activation among others in brain tissue and corresponding dose limiting side effects. To tackle the specificity problem exemplified here, we also suggest with this application the use of target cell restricted BiCos in a combination together with TAAxTCR/CD3bsAb that are directed to B7H3, a TAA expressed on tumor cells as well as on tumor vasculature. On their own these bsAbCD3 induce only weak, submitogenic T cell activation and are thus largely dependent on the presence of the BiCos. Alternatively, submitogenic CD3 stimulation might be achieved by suitable low doses of mitogenic bsAbCD3. In any case, if the two components are directed to two different target antigens the specificity of such bsAb combinations would be largely increased because their activity depends on the simultaneous presence of two different target antigens.

As summarized, there is the need for further therapeutic options which allow for an efficient targeting of tumor mass, which may be achieved by targeting such tumor associated antigens which are presented on the tumor vasculature. Hence, the present invention seeks to identify antibodies which can be used for the treatment of cancerous disorders.

### BRIEF DESCRIPTION OF THE INVENTION

Generally, and by way of brief description, the main aspects of the present invention can be described as follows:

In a first aspect, the invention pertains to a binding molecule which is at least bispecific comprising at least two first antigen binding sites and at least one second antigen binding site for use in the treatment of a disease in a subject, wherein
The at least two first antigen binding sites are capable of specifically binding to an epitope of T-cell-specific surface glycoprotein CD28; and
The at least one second antigen binding site is capable of binding to an epitope of an antigenic target protein expressed on or in a cell associated with the disease in the subject;
   wherein the treatment comprises an administration of the binding molecule to the subject.

In an alternative aspect, the invention also pertains to a endoglin binding molecule, comprising at least a heavy chain complementary determining region (CDR) 3 having the sequence RNYVTGFDY (SEQ ID NO: 16), or a sequence with no more than 3, 2, preferably no more than 1, amino acid mutations compared to this sequence; and a light chain CDR3 having the sequence HQYLSSYT (SEQ ID NO: 20), or a sequence with no more than 3, 2, preferably no more than 1, amino acid mutations compared to this sequence.

In a second aspect, the invention pertains to an isolated nucleic acid comprising a sequence encoding for the binding molecule, or for an antigen binding fragment or a monomer, such as a heavy or light chain, of the binding molecule, of any one of the first aspect.

In a third aspect, the invention pertains to a nucleic acid construct (NAC) comprising a nucleic acid of the second aspect and one or more additional sequence features permitting the expression of the encoded binding molecule, or a component of said binding molecule or (such as an antibody heavy chain or light chain) in a (host) cell.

In a fourth aspect, the invention pertains to a recombinant host cell comprising a nucleic acid or a NAC according to the second or third aspect.

In a fifth aspect, the invention pertains to a pharmaceutical composition comprising: (i) binding molecule of the first aspect, or (ii) a nucleic acid or NAC of the second or third aspect, or (iii) a recombinant host cell according to the fourth aspect, and a pharmaceutically acceptable carrier, stabiliser and/or excipient.

In a sixth aspect, the invention pertains to a kit of packages or pharmaceutical compositions, the kit comprising in separate containers: (i) an isolated binding molecule recited in any one of the preceding aspects, an isolated nucleic acid encoding the isolated binding molecule, and/or a recombinant host cell comprising such nucleic acid or isolated binding molecule; and (ii) an isolated further binding molecule recited in any one of the preceding aspects, an isolated nucleic acid encoding the isolated further binding molecule, and/or a recombinant host cell comprising such nucleic acid or isolated further binding molecule.

In a seventh aspect, the invention pertains to a component for use in medicine, wherein the component is selected from the list consisting of: (i) an binding molecule of the first aspect, or (ii) a nucleic acid or NAC of the second or third aspect, or (iii) a recombinant host cell according to the fourth aspect and (iv) a pharmaceutical composition or kit according to the fifth or sixth aspect.

### DETAILED DESCRIPTION OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In a first aspect, the invention pertains to a binding molecule which is at least bispecific comprising at least two first antigen binding sites and at least one second antigen binding site, wherein
The at least two first antigen binding sites are capable of specifically binding to an epitope of T-cell-specific surface glycoprotein CD28; and
The at least one second antigen binding site is capable of binding to an epitope of an antigenic target protein expressed on or in a cell associated with a disease.

The binding molecule of the invention is preferably for use in the treatment of a disease in a subject, wherein the treatment comprises an administration of the binding molecule to the subject.

In some specific embodiments the at least two first and/or the at least one second antigen binding site(s) are derived from an antibody or antibody like molecule, and wherein the at least two first antigen binding sites are each provided as an antigen binding fragment of an antibody which is not a F(ab′)₂ or Fab, and preferably is a single-chain construct, most preferably is a single chain Fv (scFv).

In some embodiments of the invention the binding molecule when contacted with a first cell that is a CD28 positive immune cell (such as a T-cell) in absence of a second cell expressing the antigenic target protein, does not induce CD28 signalling and preferably does not activate the immune cell (T cell).

In some preferred embodiments of the invention the antigenic target protein is selected from a protein expressed on cells associated with a proliferative disorder, a protein or other molecule associated with a pathogenic organism, such as a parasite, virus or a bacterium. For example, the antigenic target protein may be selected from endoglin, CD105, PSMA, FLT3, B7H3, or FAB.

In some embodiments, each one of the at least two first antigen binding sites may be connected directly, such as covalently or non-covalently, with the at least one second antigen binding site.

Preferred embodiments of the binding molecule comprise at least two second antigen binding sites.

In one preferred embodiment of the invention the herein disclosed binding molecule comprises exactly, and not more than, two first antigen binding sites and exactly, and not more than, one or two second antigen binding sites.

Other preferred embodiments of the invention pertain to a binding molecule of the first aspect, wherein the at least two first antigen binding sites bind the same epitope on CD28, preferably wherein the at least two antigen binding sites are identical.

It may be preferred in some embodiments, that at least one of the at least two first antigen binding sites and the at least one second antigen binding site are linked to each other by a protein-linker comprising one or more antibody-derived human constant domains, preferably of an IgG, for example they are linked via human IgG-derived hinge, CH1, CH₂, and/or CH_{3 .}

In preferred embodiments of the invention the at least two first antigen binding sites comprise an antibody heavy chain sequence and an antibody light chain sequence (preferably at least CDR1 to CDR3), each derived from, and competitively binding to the same antigen as, the C-terminal binding site (scFv) comprised in an antibody sequence shown in SEQ ID NO: 2, 3, 4, or 5 (or the N-terminal binding site comprised in an antibody sequence shown in SEQ ID NO 7/8 and 9).

In a preferred embodiment, the present invention pertains to a binding molecule, wherein the at least one second antigen binding site comprises an antibody heavy chain sequence and an antibody light chain sequence, each derived from, and competitively binding to, the same antigen (epitope) as, the N-terminal binding site comprised in an antibody composed of SEQ ID NO: 1 and 2/3 (or the C-terminal binding site comprised in an antibody sequence shown in SEQ ID NO 7/8 and 9).

In preferred embodiments of the invention, the binding molecule specifically binds to an immune cell and a cell associated with the disease, preferably wherein the immune cell is an immune cell involved with a cell-mediated immune response, such as cytotoxic immune response or a helper cell mediated immune response. Such immune cell is a cytotoxic or helper cell, such as a cell expressing CD28 and CD3 (and a TCR) and preferably is a T cell.

The antibody variable heavy and light chain sequences used for the bispecific antibodies of the invention, and which mediate a binding to CD28, are preferably in some embodiments derived from the CD28 antibody clone hu9.3.8.V1. In particular preferred is such an anti-CD28 scFv construct having the amino acid modifications in the heavy and light chain sequences as indicated in FIG. 12 or SEQ ID NO: 24. Preferably, the anti-CD28 scFv used in context of the invention should comprise a sequence having at least 90% sequence identity with SEQ ID NO: 24, preferably wherein such anti-CD28 scFv, or a sequence variant, at least comprises the amino acid Q at position 3 shown in SEQ ID NO: 24, and comprises the amino acid Q at position 16 shown in SEQ ID NO: 24; and comprises the amino acid Q at position 64 shown in SEQ ID NO: 24; and comprises the amino acid F at position 67 shown in SEQ ID NO: 24; and comprises the amino acid T at position 160 shown in SEQ ID NO: 24.

In certain alternative aspects of the invention, also provided is an anti-CD28 scFv comprising an amino acid sequence in its heavy and light chain sequences (excluding the linker) of at least 90% sequence identity, preferably 95% sequence identity, most preferably 100% sequence identity, to the sequence shown in SEQ ID NO: 24, optionally, wherein the linker sequences may be a 4GS linker of variable length, preferably wherein such anti-CD28 scFv has a sequence shown in SEQ ID NO: 24.

For the medical uses according to the invention, a subject is preferably characterized in that CD3/TCR signalling is activated by treatment, or endogenously in response to a disease-associated antigen, such as an antigenic protein. In this context, it is preferred that the treatment further comprises stimulation and/or activation of immune cells towards cells associated with the disease, such as an activation and/or stimulation of T-cells.

In one additional embodiment, the binding molecule of the invention is preferred, wherein the binding molecule comprises an equal number of first- and second antigen binding sites, and wherein one of the at least two first antigen binding sites is terminally connected (via a peptide bond) to the light chain (or alternatively the heavy chain) of one of the at least two second antigen binding sites, and wherein the other of the at least two first antigen binding sites is terminally connected (via a peptide bond) to the light chain (or alternatively the heavy chain) of the other of the at least two second antigen binding sites. Terminally connected in this embodiment shall refer to the connection to the amino acid chain directly or via a peptide linker sequence as described herein elsewhere.

The binding molecule of the invention in further preferred embodiments, comprises two antibody heavy chain sequences, and two antibody light chain sequences, and wherein
One of the at least two first antigen binding sites is covalently connected to a C-terminal end of one of the two antibody light chain sequences, and the other of the at least two first antigen binding sites is covalently connected to a C-terminal end of the other of the two antibody light chain sequences; or
One of the at least two first antigen binding sites is covalently connected to a C-terminal end of one of the two antibody heavy chain sequences, and the other of the at least two first antigen binding sites is covalently connected to a C-terminal end of the other of the two antibody heavy chain sequences.

The present invention provides a new CD28 bispecific antibody format, which comprises an IgG based antigen binding site and a (more flexible) scFv anti-CD28 binding site. In this context, the invention in preferred embodiments pertains to binding molecules, wherein the binding sites are connected either without a peptide linker or with a short peptide linker having not more than 5 amino acids, or via a long peptide linker having at least 6, preferably up to 50 amino acids, wherein further preferably the peptide linker is a (GGGGS)ₙ linker, and n is larger or equal to two, such as n=2-40, 2-30, or 2-20.

In one further embodiment of the invention, the linker may have the sequence of the formula above with n=3 to 6, preferably 4, and an additional -GGS- at the end of the sequence.

Hence, the binding molecule of the invention is preferred, wherein the at least one second antigen binding site, comprises a Fab, F(ab′)₂ or most preferably an IgG.

In another specific embodiment of the invention, the use of the binding molecule directed to CD28 is used in combination with a further binding molecule which mediates an activation of the CD3/TCR signalling axis in non-activated T cells. Hence, a preferred treatment comprises the sequential or concomitant administration of (i) a further binding molecule which is bispecific and which is capable of specifically binding to (and activating) a T cell, such as via binding to CD3 and/or a T cell receptor (TCR); or any other reagent capable of providing or enhancing signals for T cell activation such as (ii) genetically modified immune cells (heterologous or autologous T-cell) expressing an antigen receptor, such as a chimeric antigen receptor (CAR), which receptor is capable of specifically binding the antigenic target protein or (iii) vaccines providing antigenic structures from infectious agents or cancer cells (TAA or TSA) or (iv) reagents that block suppressive “second signals”, via checkpoint molecules such as PD1, PDL1, CTLA4 etc. Such reagents are termed checkpoint inhibitors.

Such a further binding molecule is at least bispecific and comprises at least one third antigen binding site and at least one fourth antigen binding site, wherein
The least one third antigen binding site is capable of specifically binding to CD3 and/or a T cell receptor (TCR) (CD3/TCR); and
The at least one fourth antigen binding site is capable of specifically binding to an epitope of a further antigenic target protein expressed on or in a cell associated with the disease in the subject.

In context of the present invention which is directed to the use of the binding molecule and the further binding molecule in combination, the antigenic target protein and the further antigenic target protein are (i) identical or (ii) different but in close spatial proximity to each other, such as being expressed on the same cell associated with the disease or located in the same diseases tissue such as being expressed in the same tumor environment. In this embodiment alternative (ii) is preferred as the selection of two different antigenic proteins for both binding molecules allows a more stringent target cell restriction and tighter regulation of T cell activation for the treatment.

Diseases to be treated by the compounds/compositions and methods of the invention are preferably proliferative diseases, preferably selected from a cancer disease, such as a cancer, for example lung cancer, breast cancer, colorectal cancer, gastric cancer, hepatocellular carcinoma, pancreatic cancer, ovarian cancer, melanoma, myeloma, kidney cancer, head and neck cancer, Hodgkin lymphoma, bladder cancer or prostate cancer, in particular one selected from the list consisting of: melanoma, lung cancer (such as non-small cell lung cancer), bladder cancer (such as urothelial carcinoma), kidney cancer (such as renal cell carcinoma), head and neck cancer (such as squamous cell cancer of the head and neck) and Hodgkin lymphoma. Preferably, the proliferative disease is melanoma, or lung cancer (such as non-small cell lung cancer), preferably a cancer positive for an expression of the target antigenic protein.

Binding Molecules which Comprise Antigen Binding Proteins Targeting CD28

An “antigen binding protein” (“ABP”) as used herein means that one or more binding sites of the binding molecule of the invention are provided by a protein that specifically binds to a target antigen, such as to one or more epitope(s) displayed by or present on a target antigen. One central antigen of the ABPs of the invention is CD28 or a orthologue (or paralogue) or other variant thereof; and the ABP can, optionally bind to one or more domains of said CD28 or variant (such as the epitope(s) can be displayed by or present on one or more extracellular domains of said 28 or variant). Typically, an antigen binding protein is an antibody (or a fragment thereof), however other forms of antigen binding protein are also envisioned by the invention. For example, the ABP may be another (non-antibody) receptor protein derived from small and robust non-immunoglobulin “scaffolds”, such as those equipped with binding functions for example by using methods of combinatorial protein design (Gebauer & Skerra, 2009; Curr Opin Chem Biol, 13:245). Particular examples of such non-antibody ABPs include: Affibody molecules based on the Z domain of Protein A (Nygren, 2008; FEBS J 275:2668); Affilins based on gamma-B crystalline and/or ubiquitin (Ebersbach et al, 2007; J Mo Biol, 372:172); Affimers based on cystatin (Johnson et al, 2012; Anal Chem 84:6553); Affitins based on Sac7d from Sulfolobus acidcaldarius (Krehenbrink et al, 2008; J Mol Biol 383:1058); Alphabodies based on a triple helix coiled coil (Desmet et al, 2014; Nature Comms 5:5237); Anticalins based on lipocalins (Skerra, 2008; FEBS J 275:2677); Avimers based on A domains of various membrane receptors (Silverman et al, 2005; Nat Biotechnol 23:1556); DARPins based on an ankyrin repeat motif (Strumpp et al, 2008; Drug Discov Today, 13:695); Fynomers based on an SH3 domain of Fyn (Grabulovski et al, 2007; J Biol Chem 282:3196); Kunitz domain peptides based on Kunitz domains of various protease inhibitors (Nixon et al, Curr opin Drug Discov Devel, 9:261) and Centyrins and Monobodies based on a 10th type III domain of fibronectin (Diem et al., 2014; Protein Eng Des Sel 27:419 doi: 10.1093/protein/gzu016; Koide & Koide, 2007; Methods Mol Biol 352:95).

The term “epitope” includes any determinant capable of being bound by an antigen binding protein, such as an antibody. An epitope is a region of an antigen that is bound by an antigen binding protein that targets that antigen, and when the antigen is a protein, includes specific amino acids that bind the antigen binding protein (such as via an antigen binding domain of said protein). Epitope determinants can include chemically active surface groupings of molecules such as amino acids, sugar side chains, phosphoryl or sulfonyl groups, and can have specific three-dimensional structural characteristics, and/or specific charge characteristics. Generally, antigen binding proteins specific for a particular target antigen will preferentially recognize an epitope on the target antigen in a complex mixture of proteins and/or macromolecules.

An antigen binding protein is “specific” when it binds to one antigen (such as CD28, CD3, endoglin, etc; eg human CD28) more preferentially (eg, more strongly or more extensively) than it binds to a second antigen. The term “specifically binds” (or “binds specifically” and the like) used herein in the context of an ABP means that said ABP will preferentially bind to the desired antigen than to bind to other proteins (or other molecules), such as preferentially binding to such compared to one or more of other Immunoglobulin (Ig) superfamily genes. Therefore, preferably, the binding affinity of the ABP to the one antigen (e.g. CD28) is at least 2-fold, 5-fold, at least 10-fold, at least 20-fold, at least 50-fold, at least 100-fold, at least 200-fold, at least 500-fold, at least 1000-fold, at least 2000-fold, at least 5000-fold, at least 10000-fold, at least 105-fold or even at least 106-fold, most preferably at least 2-fold, compared to its affinity to the other targets (e.g. unrelated proteins such as mouse or human Fc domain, or streptavidin).

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073.

In calculating percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, WI). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm.

A standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) may also be used by the algorithm.

Examples of parameters that can be employed in determining percent identity for polypeptides or nucleotide sequences using the GAP program are the following: (i) Algorithm: Needleman et al., 1970, J. Mol. Biol. 48:443-453; (ii) Comparison matrix: BLOSUM 62 from Henikoff et al., 1992, supra; (iii) Gap Penalty: 12 (but with no penalty for end gaps); (iv) Gap Length Penalty: 4; (v) Threshold of Similarity: 0.

A preferred method of determining similarity between a protein or nucleic acid and (or between) human CD28, or a binding molecule of the invention, is that provided by the Blast searches supported at Uniprot supra (e.g., http://www.uniprot.org/uniprot); in particular for amino acid identity, those using the following parameters: Program: blastp; Matrix: blosum62; Threshold: 10; Filtered: false; Gapped: true; Maximum number of hits reported: 250.

Certain alignment schemes for aligning two amino acid sequences may result in matching of only a short region of the two sequences, and this small, aligned region may have very high sequence identity even though there is no significant relationship between the two full-length sequences. Accordingly, the selected alignment method (GAP program) can be adjusted if so desired to result in an alignment that spans at least about 10, 15, 20, 25, 30, 35, 40, 45, 50 or other number of contiguous amino acids of the target polypeptide or region thereof.

In particular embodiments, an ABP of the invention can preferentially comprise at least one complementarity determining region (CDR), such as one from an antibody (in particular from a human antibody), and in particular embodiments the ABP can comprise a CDR having an amino acid sequence with at least 80%, 85%, 90% or 95% sequence identity to (preferably, at least 90% sequence identity to), or having no more than three or two, preferably no more than one amino acid substitution(s), deletion(s) or insertion(s) compared to, a CDR sequence as comprised in an antibody sequence shown in SEQ ID NO: 1-11.

The term “complementarity determining region” (or “CDR” or “hypervariable region”), as used herein, refers broadly to one or more of the hyper-variable or complementarily determining regions (CDRs) found in the variable regions of light or heavy chains of an antibody. See, for example: “IMGT”, Lefranc et al, 20003, Dev Comp Immunol 27:55; Honegger & Plückthun, 2001, J Mol Biol 309:657, Abhinandan & Martin, 2008, Mol Immunol 45:3832, Kabat, et al. (1987): Sequences of Proteins of Immunological Interest National Institutes of Health, Bethesda, Md. These expressions include the hypervariable regions as defined by Kabat et al (1983) Sequences of Proteins of Immunological Interest, US Dept of Health and Human Services, or the hypervariable loops in 3-dimensional structures of antibodies (Chothia and Lesk, 1987; J Mol Biol 196:901). The CDRs in each chain are held in close proximity by framework regions and, with the CDRs from the other chain, contribute to the formation of the antigen-binding site. Within the CDRs there are select amino acids that have been described as the selectivity determining regions (SDRs) which represent the critical contact residues used by the CDR in the antibody-antigen interaction. (Kashmiri, 2005; Methods 36:25).

An ABP of the invention may, alternatively or as well as a CDR3 sequence, comprise at least one CDR1, and/or at least one CDR2 (such as one from an antibody, in particular from a human antibody). Preferably, and ABP of the invention comprises at least one such CDR3, as well as at least one such CDR1 and at least one such CDR2, more preferably where each of such CDRs having an amino acid sequence with at least 80%, 85%, 90% or 95% (preferably at least 90%) sequence identity to, or having no more than three or two, preferably no more than one amino acid substitution(s), deletion(s) or insertion(s) compared to, a sequence selected from the corresponding (heavy and light chain) CDR1, CD2 and CD3 sequences comprised in any of the sequences shown in SEQ ID NO: 1 to 12.

In particular embodiments, an ABP of the invention can be an antibody or an antigen binding fragment thereof.

As used herein, the term “antibody” may be understood in the broadest sense as any immunoglobulin (Ig) that enables binding to its epitope. An antibody as such is a species of an ABP. Full length “antibodies” or “immunoglobulins” are generally heterotetrameric glycoproteins of about 150 kDa, composed of two identical light and two identical heavy chains. Each light chain is linked to a heavy chain by one covalent disulphide bond, while the number of disulphide linkages varies between the heavy chain of different immunoglobulin isotypes. Each heavy and light chain also has regularly spaced intrachain disulphide bridges. Each heavy chain has an amino terminal variable domain (VH) followed by three carboxy terminal constant domains (CH). Each light chain has a variable N-terminal domain (VL) and a single C-terminal constant domain (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to cells or factors, including various cells of the immune system (e.g., effector cells) and the first component (C1q) of the classical complement system. Other forms of antibodies include heavy-chain antibodies, being those which consist only of two heavy chains and lack the two light chains usually found in antibodies. Heavy-chain antibodies include the hcIgG (IgG-like) antibodies of camelids such as dromedaries, camels, llamas and alpacas, and the IgNAR antibodies of cartilaginous fishes (for example sharks). And yet other forms of antibodies include single-domain antibodies (sdAb, called Nanobody by Ablynx, the developer) being an antibody fragment consisting of a single monomeric variable antibody domain. Single-domain antibodies are typically produced from heavy-chain antibodies, but may also be derived from conventional antibodies.

Antibodies (or those from which fragments thereof can be isolated) can include, for instance, chimeric, humanized, (fully) human, or hybrid antibodies with dual or multiple antigen or epitope specificities, antibody fragments and antibody sub-fragments, e.g., Fab, Fab′ or F(ab′)₂ fragments, single chain antibodies (scFv) and the like (described below), including hybrid fragments of any immunoglobulin or any natural, synthetic or genetically engineered protein that acts like an antibody by binding to a specific antigen to form a complex.

Accordingly, in certain embodiments an ABP of the invention can comprise an antibody heavy chain, or an antigen binding fragment thereof, and/or an antibody light chain, or an antigen binding fragment thereof.

In further embodiments, an ABP of the invention can comprise an antibody heavy chain variable region, or an antigen binding fragment thereof, and/or an antibody light chain variable region, or an antigen binding fragment thereof, and in yet further embodiments, an ABP of the invention can comprise an antibody heavy chain variable region CDR1, CDR2, and CDR3, and/or an antibody light chain variable region CDR1, CDR2, and CDR3.

The present invention pertains to a binding molecule which is “bispecific” or “bifunctional”, and preferably is an ABP that has two different epitope/antigen binding domains (or “sites”), and accordingly has binding specificities for two different target epitopes. These two epitopes may be epitopes of the same antigen or, as preferred in the present invention, of different antigens, such as the different antigens endoglin and CD3/TCR.

A “bispecific ABP”, may be an ABP that binds one antigen or epitope with one of two or more binding arms, defined by a first pair of heavy and light chain or of main and shorter/smaller chain, and binds a different antigen or epitope on a second arm, defined by a second pair of heavy and light chain or of main and smaller chain. Such an embodiment of a bispecific ABP has two distinct antigen binding arms, in both specificity and CDR sequences. Typically, a bispecific ABP is monovalent for each antigen it binds to, that is, it binds with only one arm to the respective antigen or epitope. However, bispecific antibodies can also be dimerized or multimerized, which is preferred in context of the present invention. For example, in the dimeric IgGsc format as described herein, the antibody has two binding sites for each antigen (FIG. 3A-F). A bispecific antibody may be a hybrid ABP, which may have a first binding region that is defined by a first light chain variable region and a first heavy chain variable region, and a second binding region that is defined by a second light chain variable region and a second heavy chain variable region. It is envisioned by the invention that one of these binding regions may be defined by a heavy/light chain pair. In the context of the present invention the bispecific binding molecule may have a second antigen binding site, defined by variable regions of a main chain and a smaller chain, and a first, different binding site defined by a variable region of a scFv fragment that is included in the main chain of the binding molecule.

Methods of making a bispecific ABP are known in the art, e.g. chemical conjugation of two different monoclonal antibodies or for example, also chemical conjugation of two antibody fragments, for example, of two Fab fragments. Alternatively, bispecific ABPs are made by quadroma technology, that is by fusion of the hybridomas producing the parental antibodies. Because of the random assortment of H and L chains, a potential mixture of ten different antibody structures are produced of which only one has the desired binding specificity.

The bispecific ABP of the invention can act as a monoclonal antibody (mAb) with respect to each target. In some embodiments the antibody is chimeric, humanized or fully human. A bispecific ABP may for example be a bispecific tandem single chain Fv, a bispecific Fab2, or a bispecific diabody.

On the basis of the domains included in an ABP of the invention the bispecific ABP of the invention may comprise a Fab fragment, which may generally include a hinge region, a CH2 domain and a single chain Fv fragment. Such bispecific ABPs are termed “Fabsc”-ABPs and have been described for the first time in International patent application WO 2013/092001. More specifically, a “Fabsc” format ABP as used here typically refers to a bispecific ABP of the invention having a Fab fragment, which generally includes a hinge region, which is at the C-terminus of the Fab fragment linked to the N-terminus of a CH2 domain, of which the C-terminus is in turn linked to the N-terminus of a scFv fragment. Such a “Fabsc” does not or does not essentially comprise a CH3 domain. In this context, “not comprising” or “not essentially comprising” means that the ABP does not comprise a full length CH3 domain. It preferably means that the ABP comprises 10 or less, preferably 5 or less, preferably 3 or even less amino acids of the CH3 domain.

In accordance with the publication of Coloma and Morrison (Nat Biotechnol 15:159-63, 1997), a bispecific ABP of the invention may have a Fab fragment, which may generally include a hinge region, a CH2 domain, a CH3 domain, generally arranged C-terminally of the CH2 domain, and a single chain Fv fragment. Such a molecule is also referred to herein as an “IgGsc” format ABP and means a bispecific ABP of the invention having a Fab fragment, which generally includes a hinge region, which is at the C-terminus of the Fab fragment typically linked to the N-terminus of a CH2 domain, of which the C-terminus is in turn typically linked to the N-terminus of a CH3 domain, of which the C-terminus is in turn typically linked to the N-terminus of a scFv fragment. An illustrative example of an IgGsc format ABP is shown in FIG. 3. Such bispecific ABP format is preferred in context of the present invention. Such IgGsc formats occur when a.

In addition, or alternatively, an “IgGsc”ABP of the invention may also have an “Fc-attenuated” CH2 domain (that includes the hinge region). This “Fc-attenuation” is achieved by deleting and/or substituting (mutating) at least one of selected amino acid residues in the CH2 domain that are able to mediate binding to an Fc-receptor. In illustrative embodiments, the at least one amino acid residue of the hinge region or the CH2 domain that is able to mediate binding to Fc receptors and that is lacking or mutated, is selected from the group consisting of sequence position 228, 230, 231, 232, 233, 234, 235, 236, 237, 238, 265, 297, 327, and 330 (numbering of sequence positions according to the EU-index). In an illustrative example, such an Fc-attenuated ABP may contain at least one mutation selected from the group consisting of a deletion of amino acid 228, a deletion of amino acid 229, a deletion of amino acid 230, a deletion of amino acid 231, a deletion of amino acid 232, a deletion of amino acid 233, a substitution Glu233→Pro, a substitution Leu234→Val, a deletion of amino acid 234, a substitution Leu235→Ala, a deletion of amino acid 235, a deletion of amino acid 236, a deletion of amino acid 237, a deletion of amino acid 238, a substitution Asp265→Gly, a substitution Asn297→Gln, a substitution Ala327→Gln, and a substitution Ala330→Ser (numbering of sequence positions according to the EU-index, see in respect, for example, also FIG. 10 and FIG. 1P of International patent application WO 2013/092001). In the case of bispecific antibodies that activate T cells, e.g. against tumor cells, Fc-attenuation may be desired to prevent binding of the antibodies to Fc-receptor carrying cells which may lead to undesirable off-target activation of T cells.

In one preferred embodiment of the invention the CD28 costimulatory antibodies are Fc silenced and half time optimized by combining the so-called LALA Fc mutant and YTE mutants. Both modifications are well known in the art and for example in detail explained in Saunders KO. Conceptual Approaches to Modulating Antibody Effector Functions and Circulation Half-Life. Front Immunol. 2019;10:1296. Published 2019 Jun. 7. doi:10.3389/fimmu.2019.01296, incorporated herein in its entirety. Generally, the mutations are found in the human CH domains. YTE is characterized by the mutations Met252Tyr/Ser254Thr/Thr256Glu (see also Dall'acqua WF, Woods RM, Ward ES, Palaszynski SR, Patel NK, Brewah YA, et al. . Increasing the affinity of a human IgG1 for the neonatal Fc receptor: biological consequences. J Immunol. (2002) 169:5171-80. 10.4049/jimmunol 1.169.9.5171). The LALA modification is Leu234Ala/Leu235Ala (see also Hezareh M, Hessell AJ, Jensen RC, van de Winkel JG, Parren PW. Effector function activities of a panel of mutants of a broadly neutralizing antibody against human immunodeficiency virus type 1. J Virol. 2001;75(24):12161-12168. doi:10.1128/ JVI.75.24.12161-12168.2001).

The ABP of the invention therefore in a preferred embodiment comprises a combination of both LALA and YTE modifications, and as such a sequence of the CH domains shown in SEQ ID NO: 12 and 13 respectively.

The IgGsc format for the CD28 costimulatory antibodies of the invention is surprisingly effective, since unlike typically known smaller scFv only based bispecific antibodies suh as BiTE antibodies, were shown as less favourable for CD28 use due to their reduced half life and generally observed multimerization capability. The latter being complicated to control and probably bearing a risk of inducing superagonistic effects. On the other side, IgG-based formats for CD28 having superagonistic activity rendering a cell-restriction impossible. The herein suggested use of IgGsc as a CD28 bispecific format, were the CD28 binding sites are provided as the scFv in the IgGsc, was surprisingly effective in activating T cells in a target cell restricted manner, while still being only co-stimulatory and not superagonistic.

The antibody formats IgGsc have both in common that the N-terminal targeting part consists of “physiological” Fab- or Fab2 regions, respectively, thereby avoiding the use of single chain moieties in this part of the molecule. If these formats are to be used for target cell restricted T cell activation, attenuation of Fc receptor (FcR) binding may be employed (if wanted or required) to prevent FcR mediated activation. This can be achieved e.g. by introduction of defined and well-known mutations in the CH2 domain of the molecule as described in above and also in International patent application WO 2013/092001 and in Armour et al. Eur J Immunol 1999; 29:2613. Accordingly, also an IgGsc ABP of the invention may have a CH2 domain (including the hinge region) in which at least one amino acid residue of the hinge region or the CH2 domain that is able to mediate binding to Fc receptors is lacking or mutated. As explained above, this residue in the CH2 and hinge region, respectively, may be selected from the group consisting of sequence position 228, 230, 231, 232, 233, 234, 235, 236, 237, 238, 265, 297, 327, and 330 (numbering of sequence positions according to the EU-index). However, due to the presence of the CH3 domain in the IgGsc molecule, two individual molecules will (spontaneously) homodimerize via the CH3 domain to form a tetravalent molecule (see again FIG. 1B in this respect). Thus, it is not necessary to delete or mutate the cysteine residues at sequence position 226 and/or sequence position 229 of the hinge region. Thus, such a tetrameric IgGsc ABP of the invention may have a cysteine residue at sequence position 226 and/or at sequence position 229 of one of the respective hinge domain, in line with the Kabat numbering [EU-Index].

In a preferred embodiment, the invention pertains to an isolated binding molecule which is an ABP:
comprises two antibody heavy chain sequence shown in SEQ ID NO: 4 or 5, or a sequence having at least 80%, 85%, 90% or 95% (preferably at least 90%) sequence identity to any of these sequences, and two antibody light chain sequence shown in SEQ ID NO: 6, or a sequence having at least 80%, 85%, 90% or 95% (preferably at least 90%) sequence identity to this sequence; in each case independently, optionally with no more than ten, nine, preferably no more than eight, seven, six, five, four, or three or two, preferably no more than one, amino acid substitution(s), insertion(s) or deletion(s) compared to these sequences; or
comprises two antibody heavy chain sequences shown in SEQ ID NO: 1, or a sequence having at least 80%, 85%, 90% or 95% (preferably at least 90%) sequence identity to this sequence, and two antibody light chain sequence shown in SEQ ID NO: 2 or 3, or a sequence having at least 80%, 85%, 90% or 95% (preferably at least 90%) sequence identity to these sequences; in each case independently, optionally with no more than ten, nine, preferably no more than eight, seven, six, five, four, or three or two, preferably no more than one, amino acid substitution(s), insertion(s) or deletion(s) compared to these sequences; or
comprises two antibody heavy chain sequence shown in SEQ ID NO: 7 or 8, or a sequence having at least 80%, 85%, 90% or 95% (preferably at least 90%) sequence identity to any of these sequences, and two antibody light chain sequence shown in SEQ ID NO: 9, or a sequence having at least 80%, 85%, 90% or 95% (preferably at least 90%) sequence identity to this sequence; in each case independently, optionally with no more than ten, nine, preferably no more than eight, seven, six, five, four, or three or two, preferably no more than one, amino acid substitution(s), insertion(s) or deletion(s) compared to these sequences; or

In a alternative aspect, the invention pertains to an isolated binding molecule which is an ABP specifically binding endoglin, wherein the ABP:
comprises two antibody heavy chain sequence shown in SEQ ID NO: 1, or a sequence having at least 80%, 85%, 90% or 95% (preferably at least 90%) sequence identity to this sequence, and two antibody light chain sequence shown in SEQ ID NO: 6, or a sequence having at least 80%, 85%, 90% or 95% (preferably at least 90%) sequence identity to this sequence; in each case independently, optionally with no more than ten, nine, preferably no more than eight, seven, six, five, four, or three or two, preferably no more than one, amino acid substitution(s), insertion(s) or deletion(s) compared to these sequences.

In a preferred embodiment of the above ABP based binding molecules, the CDR regions are identical to the corresponding CDR sequences of the reference SEQ ID NO.

In line with the disclosure of the bispecific ABPs the invention further suggests the use of additional (further) binding molecules having an antigen binding site that specifically binds to another receptor on an immune cell such as a T cell or an NK cells, as well as to an antigen on the target cell. This receptor present on the immune cell maybe a receptor that is capable of activating the immune cell or of stimulating an immune response of the immune cell. The evoked immune response may preferably be a cytotoxic immune response. Such a suitable receptor may, for example, be CD3, the antigen specific T cell receptor (TCR), CD16, NKG2D, 0x40, 4-1 BB, CD2, CD5, programmed cell death protein 1 (PD-1) and CD95. Particularly preferred is an ABP in which the second binding site binds to CD3, TCR or CD16.

The term “isolated” as used herein in the context of a protein, such as an ABP (an example of which could be an antibody), refers to a protein that is purified from proteins or polypeptides or other contaminants that would interfere with its therapeutic, diagnostic, prophylactic, research or other use. An isolated ABP according to the invention may be a recombinant, synthetic or modified (non-natural) ABP. The term “isolated” as used herein in the context of a nucleic acid or cells refers to a nucleic acid or cells that is/are purified from DNA, RNA, proteins or polypeptides or other contaminants (such as other cells) that would interfere with its therapeutic, diagnostic, prophylactic, research or other use, or it refers to a recombinant, synthetic or modified (non-natural) nucleic acid. Preferably an isolated ABP or nucleic acid or cells is/are substantially pure. In this context, a “recombinant” protein or nucleic acid is one made using recombinant techniques. Methods and techniques for the production of recombinant nucleic acids and proteins are well known in the art.

The term “isolated” as used herein in the context of a binding molecule, such as an ABP (an example of which could be an antibody), refers to a protein that is purified from proteins or polypeptides or other contaminants that would interfere with its therapeutic, diagnostic, prophylactic, research or other use. An isolated ABP according to the invention may be a recombinant, synthetic or modified (non-natural) ABP. The term “isolated” as used herein in the context of a nucleic acid or cells refers to a nucleic acid or cells that is/are purified from DNA, RNA, proteins or polypeptides or other contaminants (such as other cells) that would interfere with its therapeutic, diagnostic, prophylactic, research or other use, or it refers to a recombinant, synthetic or modified (non-natural) nucleic acid. Preferably an isolated ABP or nucleic acid or cells is/are substantially pure. In this context, a “recombinant” protein or nucleic acid is one made using recombinant techniques. Methods and techniques for the production of recombinant nucleic acids and proteins are well known in the art.

In one embodiment, an ABP of the invention is a polyclonal antibody (mixture), or the antigen binding fragment is a fragment of a polyclonal antibody (mixture).

In an alternative, and preferred, embodiment of all ABPs of the invention, the ABP is an antibody or an antigen binding fragment thereof, and the antibody is a monoclonal antibody, or wherein the antigen binding fragment is a fragment of a monoclonal antibody.

The term “monoclonal antibody” or “mAb” as used herein refers to an antibody obtained from a population of substantially identical antibodies based on their amino acid sequence. Monoclonal antibodies are typically highly specific. Furthermore, in contrast to conventional (polyclonal) antibody preparations which typically include different antibodies directed against different determinants (e.g. epitopes) of an antigen, each mAb is typically directed against a single determinant on the antigen. In addition to their specificity, mAbs are advantageous in that they can be synthesized by cell culture (hybridomas, recombinant cells or the like) uncontaminated by other immunoglobulins. The mAbs herein include for example chimeric, humanized or human antibodies or antibody fragments.

Monoclonal antibodies in accordance with the present invention may be prepared by methods well known to those skilled in the art. For example, mice, rats or rabbits may be immunized with an antigen of interest together with adjuvant. Splenocytes are harvested as a pool from the animals that are administered several immunisations at certain intervals with test bleeds performed to assess for serum antibody titers. Splenocytes are prepared that are either used immediately in fusion experiments or stored in liquid nitrogen for use in future fusions. Fusion experiments are then performed according to the procedure of Stewart & Fuller, J. Immunol. Methods 1989, 123:45-53. Supernatants from wells with growing hybrids are screened by eg enzyme-linked immunosorbent assay (ELISA) for mAb secretors. ELISA-positive cultures are cloned either by limiting dilutions or fluorescence-activated cell sorting, typically resulting in hybridomas established from single colonies. The ability of an antibody, including an antibody fragment or sub-fragment, to bind to a specific antigen can be determined by binding assays known in the art, for example, using the antigen of interest as the binding partner.

In a further preferred embodiment, an ABP of the invention is an antibody or an antigen binding fragment thereof, wherein the antibody is a human antibody a humanised antibody or a chimeric-human antibody, or wherein the antigen binding fragment is a fragment of a human antibody a humanised antibody or a chimeric-human antibody.

Human antibodies can also be derived by in vitro methods. Suitable examples include but are not limited to phage display (CAT, Morphosys, Dyax, Biosite/Medarex, Xoma, Yumab, Symphogen, Alexion, Affimed) and the like. In phage display, a polynucleotide encoding a single Fab or Fv antibody fragment is expressed on the surface of a phage particle (see e.g., Hoogenboom et al., J. Mol. Biol., 227: 381 (1991); Marks et al., J Mol Biol 222: 581 (1991); U.S. Pat. No. 5,885,793). Phage are “screened” to identify those antibody fragments having affinity for target. Thus, certain such processes mimic immune selection through the display of antibody fragment repertoires on the surface of filamentous bacteriophage, and subsequent selection of phage by their binding to target. In certain such procedures, high affinity functional neutralizing antibody fragments are isolated. A complete repertoire of human antibody genes may thus be created by cloning naturally rearranged human V genes from peripheral blood lymphocytes (see, e.g., Mullinax et al., Proc Natl Acad Sci (USA), 87: 8095-8099 (1990)) or by generating fully synthetic or semi-synthetic phage display libraries with human antibody sequences (see Knappik et al 2000; J Mol Biol 296:57; de Kruif et al, 1995; J Mol Biol 248):97).

The antibodies described herein may alternatively be prepared through the utilization of the XenoMouse® technology. Such mice are capable of producing human immunoglobulin molecules and antibodies and are deficient in the production of murine immunoglobulin molecules and antibodies. In particular, a preferred embodiment of transgenic production of mice and antibodies is disclosed in U.S. patent application Ser. No. 08/759,620, filed Dec. 3, 1996 and International Patent Application Nos. WO 98/24893, published Jun. 11, 1998 and WO 00/76310, published Dec. 21, 2000. See also Mendez et al., Nature Genetics, 15:146-156 (1997). Through the use of such technology, fully human monoclonal antibodies to a variety of antigens have been produced. Essentially, XenoMouse® lines of mice are immunized with an antigen of interest. e.g. CD28, endoglin etc., lymphatic cells (such as B-cells) are recovered from the hyper-immunized mice, and the recovered lymphocytes are fused with a myeloid-type cell line to prepare immortal hybridoma cell lines. These hybridoma cell lines are screened and selected to identify hybridoma cell lines that produce antibodies specific to the antigen of interest. Other “humanised” mice are also commercially available: eg, Medarex—HuMab mouse, Kymab—Kymouse, Regeneron—Velocimmune mouse, Kirin—TC mouse, Trianni—Trianni mouse, OmniAb—OmniMouse, Harbour Antibodies—H2L2 mouse, Merus—MeMo mouse. Also are available are “humanised” other species: rats: OmniAb—OmniRat, OMT—UniRat. Chicken: OmniAb—OmniChicken.

The term “humanised antibody” according to the present invention refers to immunoglobulin chains or fragments thereof (such as Fab, Fab′, F(ab′)2, Fv, or other antigen-binding sub-sequences of antibodies), which contain minimal sequence (but typically, still at least a portion) derived from non-human immunoglobulin. For the most part, humanised antibodies are human immunoglobulins (the recipient antibody) in which CDR residues of the recipient antibody are replaced by CDR residues from a non-human species immunoglobulin (the donor antibody) such as a mouse, rat or rabbit having the desired specificity, affinity and capacity. As such, at least a portion of the framework sequence of said antibody or fragment thereof may be a human consensus framework sequence. In some instances, Fv framework residues of the human immunoglobulin need to be replaced by the corresponding non-human residues to increase specificity or affinity. Furthermore, humanised antibodies can comprise residues which are found neither in the recipient antibody nor in the imported CDR or framework sequences. These modifications are made to further refine and maximise antibody performance. In general, the humanised antibody will comprise substantially all of at least one, and typically at least two, variable domains, in which all or substantially all of the CDR regions correspond to those of a non-human immunoglobulin and all or substantially all of the framework regions are those of a human immunoglobulin consensus sequence. The humanised antibody optimally also will comprise at least a portion of an immunoglobulin constant region, typically that of a human immunoglobulin, which (eg human) immunoglobulin constant region may be modified (eg by mutations or glycoengineering) to optimise one or more properties of such region and/or to improve the function of the (eg therapeutic) antibody, such as to increase or reduce Fc effector functions or to increase serum half-life. Exemplary such Fc modification (for example, Fc engineering or Fc enhancement) are described elsewhere herein.

The term “chimeric antibody” according to the present invention refers to an antibody whose light and/or heavy chain genes have been constructed, typically by genetic engineering, from immunoglobulin variable and constant regions which are identical to, or homologous to, corresponding sequences of different species, such as mouse and human. Alternatively, variable region genes derive from a particular antibody class or subclass while the remainder of the chain derives from another antibody class or subclass of the same or a different species. It covers also fragments of such antibodies. For example, a typical therapeutic chimeric antibody is a hybrid protein composed of the variable or antigen-binding domain from a mouse antibody and the constant or effector domain from a human antibody, although other mammalian species may be used.

In particular of such embodiments, an ABP of the invention comprises an antigen binding domain of an antibody wherein the antigen binding domain is of a human antibody. Preferably, ABP comprises an antigen binding domain of an antibody or an antigen binding fragment thereof, which is a human antigen binding domain; (ii) the antibody is a monoclonal antibody, or wherein the antigen binding fragment is a fragment of a monoclonal antibody; and (iii) the antibody is a human antibody or a humanised antibody, or wherein the antigen binding fragment is a fragment of a human antibody, a humanised antibody or a chimeric-human antibody.

Light chains of human antibodies generally are classified as kappa and lambda light chains, and each of these contains one variable region and one constant domain. Heavy chains are typically classified as mu, delta, gamma, alpha, or epsilon chains, and these define the antibody's isotype as IgM, IgD, IgG, IgA, and IgE, respectively. Human IgG has several subtypes, including, but not limited to, IgG1, IgG2, IgG3, and IgG4. Human IgM subtypes include IgM, and IgM2. Human IgA subtypes include IgA1 and IgA2. In humans, the IgA and IgD isotypes contain four heavy chains and four light chains; the IgG and IgE isotypes contain two heavy chains and two light chains; and the IgM isotype contains ten or twelve heavy chains and ten or twelve light chains. Antibodies according to the invention may be IgG, IgE, IgD, IgA, or IgM immunoglobulins.

In some embodiments, the ABP of the invention is an IgG antibody or fragment thereof. In some embodiments, the ABP of the invention is an IgE antibody or fragment thereof. In some embodiments, the ABP of the invention is an IgD antibody or fragment thereof. In some embodiments, the ABP of the invention is an IgA antibody or fragment thereof. In some embodiments, the ABP of the invention is an IgM antibody or fragment thereof. Preferably the ABP of the invention is, comprises or is derived from an IgG immunoglobulin or fragment thereof; such as a human, human-derived IgG immunoglobulin, or a rabbit- or rat-derived IgG, and/or an IgG2 immunoglobulin, or fragment thereof. When the ABP of the invention is, comprises or is derived from a rat-derived IgG, then preferably, the ABP is, comprises or is derived from, a rat IgG2a or IgG2b immunoglobulin. When the ABP of the invention is, comprises or is derived from a human-derived IgG, then more preferably, the ABP of the invention is, comprises or is derived from a human IgG1, IgG2 or IgG4, most preferably, the ABP of the invention is, comprises or is derived from a human IgG1 or IgG2

Accordingly, in particular embodiments of the invention, an ABP is an antibody wherein the antibody is an IgG, IgE, IgD, IgA, or IgM immunoglobulin; preferably an IgG immunoglobulin.

An ABP of the invention, where comprising at least a portion of an immunoglobulin constant region (typically that of a human immunoglobulin) may have such (eg human) immunoglobulin constant region modified—for example eg by glycoengineering or mutations—to optimise one or more properties of such region and/or to improve the function of the (eg therapeutic) antibody, such as to increase or reduce Fc effector functions or to increase serum half-life.

ABPs of the invention, in particular those useful in the present methods include antibodies that induce antibody-dependent cytotoxicity (ADCC) of endoglin-expressing cells. The ADCC of an anti-endoglin antibody can be improved by using antibodies that have low levels of or lack fucose. Antibodies lacking fucose have been correlated with enhanced ADCC (antibody-dependent cellular cytotoxicity) activity, especially at low doses of antibody (Shields et ah, 2002, J. Biol. Chem. 277:26733-26740; Shinkawa et ah, 2003, J. Biol. Chem. 278:3466).

Methods of preparing fucose-less antibodies or antibodies with reduced fucose levels include growth in rat myeloma YB2/0 cells (ATCC CRL 1662). YB 2/0 cells express low levels of FUT8 mRNA, which encodes an enzyme (.alpha. 1,6- fucosyltransferase) necessary for fucosylation of polypeptides.

Alternatively, during the expression of such antibodies, an inhibitor against an enzyme relating to the modification of a sugar chain may be used, including: tunicamycin which selectively inhibits formation of GlcNAc-P-P-Dol which is the first step of the formation of a core oligosaccharide which is a precursor of an N- glycoside-linked sugar chain, castanospermin and W-methyl-1-deoxynojirimycin which are inhibitors of glycosidase I, kifunensine which is an inhibitor of mannosidase I, bromocondulitol which is an inhibitor of glycosidase II, 1-deoxynojirimycin and 1 ,4-dioxy-1,4-imino-D-mannitol which are inhibitors of mannosidase I, swainsonine which is an inhibitor of mannosidase II, swainsonine which is an inhibitor of mannosidase II and the like. Examples of an inhibitor specific for a glycosyltransf erase include deoxy derivatives of substrates against N-acetylglucosamine transferase V (GnTV) and the like. Also it is known that 1-deoxynojirimycin inhibits synthesis of a complex type sugar chain and increases the ration of high mannose type and hybrid type sugar chains (Glycobiology series 2-Destiny of Sugar Chain in Cell, edited by Katsutaka Nagai, Senichiro Hakomori and Akira Kobata, 1993).

Based on these data, several cell lines have been genetically engineered to produce antibodies containing no or low levels of fucose (Mori et al, 2004; Yamane-Ohnuki et al., 2004) to engineer the glycosylation patterns of IgG in order to select therapeutic monoclonal antibodies exhibiting particular profiles of Fc-gamma-R engagement that could be used in various pathologies.

Umana et al. and Davis et al. showed that an IgG1 antibody engineered to contain increasing amounts of bisected complex oligosaccharides (bisecting A/-acetylglucosamine, GlcNAC) allows triggering a strong ADCC as compared to its parental counterpart (Umana et al., 1999; Davies et al., 2001). Second, a lack of fucose on human IgG1 N-linked oligosaccharides has been shown to improve FCGRIII binding and ADCC.

GLYCART BIOTECHNOLOGY AG (Zurich, CH) has expressed N-acetyl-glucosaminyltransferase III (GnTIII) which catalyses the addition of the bisecting GlcNac residue to the N-linked oligosaccharide, in a Chinese hamster ovary (CHO) cell line, and showed a greater ADCC of IgG1 antibody produced (WO 99/54342; WO 03/011878; WO 2005/044859).

WO20070166306 is related to the modification of an antibody anti-CD19 containing 60% N-acetylglucosamine bisecting oligosaccharides and 10% non-fucosylated N-acetylglucosamine bisecting oligosaccharides produced in a mammalian human 293T embryonal kidney cells transfected with (i) the cDNA for the anti-CD19 antibody and (ii) the cDNA for the GnTIII enzyme.

Recombinant human IgG1 produced in YB2/0 cells (Shinkawa et al., 2003; Siberil et al., 2006) or in CHO-Lec13 (Shields et al., 2002) which exhibited a low-fucose content or were deficient in fucose as compared to the same IgG1 produced in wild-type CHO cells, showed an enhanced ability to trigger cellular cytotoxicity. By contrast, a correlation between galactose and ADCC was not observed and the content of bisecting GlcNAC only marginally affected ADCC (Shinkawa et al., 2003).

By removing or supplanting fucose from the Fc portion of the antibody, KYOWA HAKKO KOGYO (Tokyo, Japan) has enhanced Fc binding and improved ADCC, and thus the efficacy of the MAb (U.S. Pat. No. 6,946,292). This improved Fc-gamma-RIIIA-dependent effector functions of low-fucosylated IgG has been shown to be independent from Fc-gamma-RI I I allelic form (Niwa et al., 2005). Moreover, it has been recently shown that the antigenic density required to induce an efficient ADCC is lower when the IgG has a low content in fucose as compared to a highly fucosylated IgG (Niwa et al., 2005)

The Laboratoire Frangais du Fractionnement et des Biotechnologies (LFB) (France) showed that the ratio Fuc/Gal in MAb oligosaccharide should be equal or lower than 0.6 to get antibodies with a high ADCC (FR 2 861080).

Cardarelli et al., 2019 produce an anti-CD19 antibody in Ms-704PF CHO cells deficient in the FUT8 gene which encodes alphal ,6-fucosyltransferase, Non-fucosylation of the antibody in this paper requires the engineering of an enzymes-deficient cell line. This paper does not consider amino acid mutations.

Herbst et al. generated a humanized IgG1 MAb MEDI-551 expressed in a fucosyltransferase-deficient producer CHO cell line This paper does not consider amino acid mutations (Herbst et al., 2010) [0048] S. Siberil et al used the rat myeloma YB2/0 cell line to produce a MAb anti RhD with a low fucose content. Whereas the MAb produced in a wild type CHO exhibited a high fucose content (81%), the same MAb produced in YB2/0 cell exhibited a lower fucose content (32%). This paper does consider amino acid mutations (Siberil et al., 2006).

Accordingly, An ABP of the invention may be prepared and/or may have one or more of the characteristics of such glycoengineering (eg afucosylated) approaches/antibodies described above.

Alternative methods for increasing ADDC activity for an ABP of the invention include mutations in an Fc portion of such ABP, particularly mutations which increase antibody affinity for an Fc-gamma-R receptor.

Accordingly, any of the ABPs of the invention described above can be produced with different isotypes or mutant isotypes to control the extent of binding to different Fc-gamma receptors. Antibodies lacking an Fc region (e.g., Fab fragments) lack binding to different Fc-gamma receptors. Selection of isotype also affects binding to different Fc-gamma receptors. The respective affinities of various human IgG isotypes for the three different Fc-gamma receptors, Fc-gamma-RI, Fc-gamma-RII, and Fc-gamma-RIII, have been determined. (See Ravetch & Kinet, Annu. Rev. Immunol. 9, 457 (1991)) Fc-gamma-RI is a high affinity receptor that binds to IgGs in monomeric form, and the latter two are low affinity receptors that bind IgGs only in multimeric form. In general, both IgG1 and IgG3 have significant binding activity to all three receptors, IgG4 to Fc-gamma-RI, and IgG2 to only one type of Fc-gamma-RII called IIaLR (see Parren et al., J. Immunol. 148, 695 (1992). Therefore, human isotype IgG1 is usually selected for stronger binding to Fc-gamma receptors is desired, and IgG2 is usually selected for weaker binding.

A correlation between increased Fc-gamma-R binding with mutated Fc has been demonstrated using targeted cytoxicity cell-based assays (Shields et ah, 2001, J. Biol. Chem. 276:6591-6604; Presta et ah, 2002, Biochem Soc. Trans. 30:487-490). Methods for increasing ADCC activity through specific Fc region mutations include the Fc variants comprising at least one amino acid substitution at a position selected from the group consisting of: 234, 235, 239, 240, 241, 243, 244, 245, 247, 262, 263, 264, 265, 266, 267, 269, 296, 297, 298, 299, 313, 325, 327, 328, 329, 330 and 332, wherein the numbering of the residues in the Fc region is that of the EU index as in Kabat (Kabat et ah, Sequences of Proteins of Immunological Interest (National Institute of Health, Bethesda, Md. 1987).

In certain specific embodiments, said Fc variants comprise at least one substitution selected from the group consisting of L234D, L234E, L234N, L234Q, L234T, L234H, L 234Y, L234I, L234V, L234F, L235D, L235S, L235N, L235Q, L235T, L235H, L235Y, L235I, L235V, L235F, S239D, S239E, S239N, S239Q, S239F, S239T, S239H, S239Y, V240I, V240A, V240T, V240M, F241W, F241L, F241Y, F241E, F241R, F243W, F243L, F243Y, F243R, F243Q, P244H, P245A, P247V, P247G, V262I, V262A, V262T, V262E, V263I, V263A, V263T, V263M, V264L, V264I, V264W, V264T, V264R, V264F, V264M, V264Y, V264E, D265G, D265N, D265Q, D265Y, D265F, D265V, D265I, D265L, D265H, D265T, V266I, V266A, V266T, V266M, S267Q, S267L, E269H, E269Y, E269F, E269R, Y296E, Y296Q, Y296D, Y296N, Y296S, Y296T, Y296L, Y296I, Y296H, N297S, N297D, N297E, A298H, T299I, T299L, T299A, T299S, T299V, T299H, T299F, T299E, W313F, N325Q, N325L, N325I, N325D, N325E, N325A, N325T, N325V, N325H, A327N, A327L, L328M, L328D, L328E, L328N, L328Q, L328F, L328I, L328V, L328T, L328H, L328A, P329F, A330L, A330Y, A330V, A330I, A330F, A330R, A330H, I332D, I332E, I332N, I332Q, I332T, I332H, I332Y and I332A, wherein the numbering of the residues in the Fc region is that of the EU index as in Kabat.

Fc variants can also be selected from the group consisting of V264L, V264I, F241W, F241L, F243W, F243L, F241L/F243L/V262I/V264I, F241W/F243W, F241W/F243W/V262A/V264A, F241L/V262I, F243L/V264I, F243L/V262I/V264W, F241Y/F243Y/V262T/V264T, F241E/F243R/V262E/V264R, F241E/F243Q/V262T/V264E, F241R/F243Q/V262T/V264R, F241E/F243Y/V262T/V264R, L328M, L328E, L328F, I332E, L3238M/I332E, P244H, P245A, P247V, W313F, P244H/P245A/P247V, P247G, V264I/I332E, F241E/F243R/V262E/V264R/I332E, F241E/F243Q/V262T/264E/I332E, F241R/F243Q/V262T/V264R/I332E, F241E/F243Y/V262T/V264R/I332E, S298A/I332E, S239E/I332E, S239Q/I332E, S239E, D265G, D265N, S239E/D265G, S239E/D265N, S239E/D265Q, Y296E, Y296Q, T299I, A327N, S267Q/A327S, S267L/A327S, A327L, P329F, A330L, A330Y, I332D, N297S, N297D, N297S/I332E, N297D/I332E, N297E/I332E, D265Y/N297D/I332E, D265Y/N297D/T299L/I332E, D265F/N297E/I332E, L328I/I332E, L328Q/I332E, I332N, I332Q, V264T, V264F, V240I, V263I, V266I, T299A, T299S, T299V, N325Q, N325L, N325I, S239D, S239N, S239F, S239D/I332D, S239D/I332E, S239D/I332N, S239D/I332Q, S239E/I332D, S239E/I332N, S239E/I332Q, S239N/I332D, S239N/I332E, S239N/I332N, S239N/I332Q, S239Q/I332D, S239Q/I332N, S239Q/I332Q, Y296D, Y296N, F241Y/F243Y/V262T/V264T/N297D/I332E, A330Y/I332E, V264I/A330Y/I332E, A330L/I332E, V264I/A330L/I332E, L234D, L234E, L234N, L234Q, L234T, L234H, L234Y, L234I, L234V, L234F, L235D, L235S, L235N, L235Q, L235T, L235H, L235Y, L235I, L235V, L235F, S239T, S239H, S239Y, V240A, V240T, V240M, V263A, V263T, V263M, V264M, V264Y, V266A, V266T, V266M, E269H, E269Y, E269F, E269R, Y296S, Y296T, Y296L, Y296I, A298H, T299H, A330V, A330I, A330F, A330R, A330H, N325D, N325E, N325A, N325T, N325V, N325H, L328D/I332E, L328E/I332E, L328N/I332E, L328Q/I332E, L328V/I332E, L328T/I332E, L328H/I332E, L328I/I332E, L328A, I332T, I332H, I332Y, I332A, S239E/V264I/I332E, S239Q/V264I/I332E, S239E/V264I/A330Y/I332E, S239E/V264I/S298A/A330Y/I332E, S239D/N297D/I332E, S239E/N297D/I332E, S239D/D265V/N297D/I332E, S239D/D265I/N297D/I332E, S239D/D265L/N297D/I332E, S239D/D265F/N297D/I332E, S239D/D265Y/N297D/I332E, S239D/D265H/N297D/I332E, S239D/D265T/N297D/I332E, V264E/N297D/I332E, Y296D/N297D/I332E, Y296E/N297D/I332E, Y296N/N297D/I332E, Y296Q/N297D/I332E, Y296H/N297D/I332E, Y296T/N297D/I332E, N297D/T299V/I332E, N297D/T299I/I332E, N297D/T299L/I332E, N297D/T299F/I332E, N297D/T299H/I332E, N297D/T299E/I332E, N297D/A330Y/I332E, N297D/S298A/A330Y/I332E, S239D/A330Y/I332E, S239N/A330Y/I332E, S239D/A330L/I332E, S239N/A330L/I332E, V264I/S298A/I332E, S239D/S298A/I332E, S239N/S298A/I332E, S239D/V264I/I332E, S239D/V264I/S298A/I332E, AND S239D/264I/A330L/I332E, wherein the numbering of the residues in the Fc region is that of the EU index as in Kabat. See also WO2004029207, incorporated by reference herein.

In particular embodiments, mutations on, adjacent, or close to sites in the hinge link region (e.g., replacing residues 234, 235, 236 and/or 237 with another residue) can be made, in all of the isotypes, to reduce affinity for Fc-gamma receptors, particularly Fc-gamma-RI receptor (see, eg U.S. Pat No. 6,624,821). Optionally, positions 234, 236 and/or 237 are substituted with alanine and position 235 with glutamate. (See, eg U.S. Pat. No. 5,624,821.) Position 236 is missing in the human IgG2 isotype. Exemplary segments of amino acids for positions 234, 235 and 237 for human IgG2 are Ala Ala Gly, Val Ala Ala, Ala Ala Ala, Val Glu Ala, and Ala Glu Ala. A preferred combination of mutants is L234A, L235E and G237A, or is L234A, L235A, and G237A for human isotype IgG1. A particular preferred ABP of the invention is an antibody having human isotype IgG and one of these three mutations of the Fc region. Other substitutions that decrease binding to Fc-gamma receptors are an E233P mutation (particularly in mouse IgG1) and D265A (particularly in mouse IgG2a). Other examples of mutations and combinations of mutations reducing Fc and/or C1q binding are E318A/K320A/R322A (particularly in mouse IgG1), L235A/E318A/K320A/K322A (particularly in mouse IgG2a). Similarly, residue 241 (Ser) in human IgG4 can be replaced, eg with proline to disrupt Fc binding.

Additional mutations can be made to a constant region to modulate effector activity. For example, mutations can be made to the IgG1 or IgG2a constant region at A330S, P331S, or both. For IgG4, mutations can be made at E233P, F234V and L235A, with G236 deleted, or any combination thereof. IgG4 can also have one or both of the following mutations S228P and L235E. The use of disrupted constant region sequences to modulate effector function is further described, eg in WO2006118,959 and WO2006036291.

Additional mutations can be made to the constant region of human IgG to modulate effector activity (see, e.g., WO200603291). These include the following substitutions: (i) A327G, A330S, P331S; (ii) E233P, L234V, L235A, G236 deleted; (iii) E233P, L234V, L235A; (iv) E233P, L234V, L235A, G236 deleted, A327G, A330S, P331S; and (v) E233P, L234V, L235A, A327G, A330S, P331S to human IgG1; or in particular, (vi) L234A, L235E, G237A, A330S and P331S (eg, to human IgG1), wherein the numbering of the residues in the Fc region is that of the EU index as in Kabat. See also WO2004029207, incorporated by reference herein.

The affinity of an antibody for the RR can be altered by mutating certain residues of the heavy chain constant region. For example, disruption of the glycosylation site of human IgG1 can reduce RR binding, and thus effector function, of the antibody (see, eg WO2006036291). The tripeptide sequences NXS and NXT, where X is any amino acid other than proline, are the enzymatic recognition sites for glycosylation of the N residue. Disruption of any of the tripeptide amino acids, particularly in the CH2 region of IgG, will prevent glycosylation at that site. For example, mutation of N297 of human IgG1 prevents glycosylation and reduces FcR binding to the antibody.

A preferred enhancement of Fc receptor binding can be achieved by introducing the Fc domain mutants of human IgG1 referred to herein generally as “SDIE”, which denote the mutations S239D/I332E.

Although activation of ADCC and CDC is often desirable for therapeutic antibodies, there are circumstances in which an ABP of the invention unable to activate effector functions is preferential (eg, an ABP of the invention that is an agnostic modulator). For these purposes IgG4 has commonly been used but this has fallen out of favour in recent years due the unique ability of this sub-class to undergo Fab-arm exchange, where heavy chains can be swapped between IgG4 in vivo. Accordingly, Fc engineering approaches can also be used to determine the key interaction sites for the Fc domain with Fc-gamma receptors and C1q and then mutate these positions, such as in an Fc of an ABP of the invention, to reduce or abolish binding. Through alanine scanning Duncan and Winter (1998; Nature 332:738) first isolated the binding site of C1q to a region covering the hinge and upper CH2 of the Fc domain. Researchers at Genmab identified mutants K322A, L234A and L235A, which in combination are sufficient to almost completely abolish Fc-gamma-R and C1q binding (Hezareh et al, 2001; J Virol 75:12161). In a similar manner MedImmune later identified a set of three mutations, L234F/L235E/P331S (dubbed™), which have a very similar effect (Oganesyan et al, 2008; Acta Crystallographica 64:700). An alternative approach is modification of the glycosylation on asparagine 297 of the Fc domain, which is known to be required for optimal FcR interaction. A loss of binding to FcRs has been observed in N297 point mutations (Tao et al, 1989; J Immunol 143:2595), enzymatically degylcosylated Fc domains (Mimura et al, 2001; J Biol Chem 276:45539), recombinantly expressed antibodies in the presence of a glycosylation inhibitor (Walker et al, 1989; Biochem J 259:347) and the expression of Fc domains in bacteria (Mazor et al 2007; Nat Biotechnol 25:563). Accordingly, the invention also includes embodiments of the ABPs in which such technologies or mutations have been used to reduce effector functions.

IgG naturally persists for a prolonged period in (eg human) serum due to FcRn-mediated recycling, giving it a typical half-life of approximately 21 days. Despite this there have been a number of efforts to engineer the pH dependant interaction of the Fc domain with FcRn to increase affinity at pH 6.0 while retaining minimal binding at pH 7.4. Researchers at PDL BioPharma identified the mutations T250Q/M428L, which resulted in an approximate 2-fold increase in IgG half-life in rhesus monkeys (Hinto et al, 2004; J Biol Chem 279:6213), and M252Y/S254T/T256E (dubbed YTE), which resulted in an approximate 4-fold increase in IgG half-life in cynomolgus monkeys (Dall'Acqua, et al 2006; J Biol Chem 281:23514). ABPs of the invention may aslo be PEGylated. PEGylation, ie chemical coupling with the synthetic polymer poly-ethylene glycol (PEG), has emerged as an accepted technology for the development of biologics that exercise prolonged action, with around 10 clinically approved protein and peptide drugs to date (Jevsevar et al., 2010; Biotechnol J 5:113). ABPs of the invention may also be subjected to PASylation, a biological alternative to PEGylation for extending the plasma half-life of pharmaceutically active proteins (Schlapschy et al, 2013; Protein Eng Des Sel 26:489; XL-protein GmbH, Germany). Accordingly, the invention also includes embodiments of the ABPs in which such technologies or mutations have been used to prolong serum half-life, especially in human serum.

“Fab fragments” are composed of one constant and one variable domain of each of the heavy and the light chains, held together by the adjacent constant region of the light chain and the first constant domain (CH1) of the heavy chain. These may be formed by protease digestion, e.g. with papain, from conventional antibodies, but similar Fab fragments may also be produced by genetic engineering. Fab fragments include Fab′, Fab and “Fab-SH” (which are Fab fragments containing at least one free sulfhydryl group).

Fab′ fragments differ from Fab fragments in that they contain additional residues at the carboxy terminus of the first constant domain of the heavy chain including one or more cysteines from the antibody hinge region. Fab′ fragments include “Fab′-SH” (which are Fab′fragments containing at least one free sulfhydryl group).

Further, antibody fragments include F(ab′)₂ fragments, which contain two light chains and two heavy chains containing a portion of the constant region between the CH1 and CH2 domains (“hinge region”), such that an interchain disulphide bond is formed between the two heavy chains. A F(ab′)₂ fragment thus is composed of two Fab′ fragments that are held together by a disulphide bond between the two heavy chains. F(ab′)₂ fragments may be prepared from conventional antibodies by proteolytic cleavage with an enzyme that cleaves below the hinge region, e.g. with pepsin, or by genetic engineering.

An “Fv region” comprises the variable regions from both the heavy and light chains, but lacks the constant regions. “Single-chain antibodies” or “scFv” are Fv molecules in which the heavy and light chain variable regions have been connected by a flexible linker to form a single polypeptide chain, which forms an antigen binding region.

An “Fc region” comprises two heavy chain fragments comprising the CH2 and CH3 domains of an antibody. The two heavy chain fragments are held together by two or more disulphide bonds and by hydrophobic interactions of the CH3 domains.

In a preferred embodiment, an ABP of the invention is an antibody wherein at least a portion of the framework sequence of said antibody or fragment thereof is a human consensus framework sequence, for example, comprises a human germline-encoded framework sequence.

In some embodiments, an ABP of the invention is modified or engineered to increase antibody-dependent cellular cytotoxicity (ADCC). As will now be understood by the person of ordinary skill, such ABPs of the invention will have particular utility in the therapy of diseases or disorders associated with cellular resistance against immune cells like CTLs (such as an CD28-positive cancer); as the ADCC mechanism (a cell-mediated immune defence whereby an effector cell of the immune system actively lyses a target cell, whose membrane-surface antigens have been bound by specific antibodies) would be enhanced in respect of the cells having resistance against immune cells like CTLs, hence leading to an increase in attachment by and/or lysis of such cells by effector cells of the immune system.

As used herein, “therapy” is synonymous with treating a disease, disorder or condition, which includes reducing symptoms of the disease, disorder or condition, inhibiting progression of the disease, disorder or condition, causing regression of the disease, disorder or condition and/or curing the disease, disorder or condition.

Various techniques to modify or engineer an ABP of the invention to increase ADCC are known (Satoh et al, 2006; Expert Opin Biol Ther 6:1161; WO2009/135181), and hence such embodiments include those wherein an ABP of the invention may be afucosylated (GlycArt Biotechnology) e.g., in which antibodies are produced in CHO cells in which the endogenous FUT8 gene has been knocked out; or the ABP may be a “Sugar-Engineered Antibody” (Seattle Genetics), e.g. in which fucose analogues are added to antibody-expressing CHO cells, resulting in a significant reduction in fucosylation. Other afucosylation approaches that may be applied to an ABP of the invention are described elsewhere herein.

Other techniques to modify or engineer an ABP of the invention to increase ADCC include mutations in a Fc portion of the ABP, (such as described in more detail elsewhere herein), in particular where one or more of residues 234, 235, 236 and/or 237, and/or residues 330, 331 of human Fc are so mutated; wherein such numbering of the residues in the Fc region is that of the EU index as in Kabat (Kabat et ah, Sequences of Proteins of Immunological Interest (National Institute of Health, Bethesda, Md. 1987).

Accordingly, in certain embodiments, the ABP of the invention is modified or engineered to increase antibody-dependent cell-mediated cytotoxicity (ADCC), preferably wherein said ABP is afucosylated and/or an Fc of said ABP is mutated (eg where an Fc is mutated using one or more of the following residue changes: L234A, L235E, G237A, A330S and/or P331S). In alternative embodiments, the ABP of the invention is modified or engineered to reduce antibody-dependent cell-mediated cytotoxicity (ADCC).

In other certain embodiments, the ABP of the invention is modified to prolong serum half-life, especially in human serum. For example, an ABP of the invention may be PEGylated and/or PASylated or has an Fc region with a T250Q/M428L or M252Y/S254T/T256E modification.

An ABP of the present invention may be mono-specific (i.e, it possesses antigen binding domain(s) that bind to only one antigen) or may be multi-specific (i.e, it possesses two or more different antigen binding domain(s) that bind to different antigens). For example, a “bi-specific”, “dual-specific” or “bifunctional” ABP or antibody is a hybrid ABP or antibody, respectively, having two different antigen binding sites. Bi-specific antigen binding proteins and antibodies are a species of multi-specific antigen binding protein antibody and can be produced by a variety of methods including, but not limited to, fusion of hybridomas or linking of Fab′ fragments (see, e.g., Songsivilai and Lachmann, 1990; Kostelny et al., 1992). The two binding sites of a bi-specific antigen binding protein or antibody will bind to two different epitopes, which can reside on the same or different protein targets.

Accordingly, in certain embodiments, the ABP of the invention is a multi-specific antibody comprising at least two antigen binding domains, wherein each antigen binding domain specifically binds to a different antigen epitope.

Preferred variants of the ABP of the invention pertain to bispecifics, which comprise the antigen binding regions of the antibodies against CD28 and a second antigen binding region directed at a disease associated antigenic protein such as endoglin. Preferably the anti-CD28 binding site is an scFv construct. One preferred construct of the invention comprises two antigen binding domains of the herein designated 7C4 antibody and two anti-CD3 binding domains, for example the aforementioned UCHT1 scFv construct.

In preferred embodiments, an ABP of the invention can comprise at least one antibody constant domain, in particular wherein at least one antibody constant domain is a CH1, CH2, or CH3 domain, or a combination thereof.

In further of such embodiments, an ABP of the invention having antibody constant domain comprises a mutated Fc region, for example for increasing interaction of the Fc region with a Fc receptor (Fc receptor on an immune effector cell (eg Saxena & Wu, 2016; Front Immunol 7:580). Examples and embodiments thereof are described elsewhere herein.

In other embodiments, an ABP of the invention may comprises an effector group and/or a labelling group.

The term “effector group” means any group, in particular one coupled to another molecule such as an antigen binding protein, that acts as a cytotoxic agent. Examples for suitable effector groups are radioisotopes or radionuclides. Other suitable effector groups include toxins, therapeutic groups, or chemotherapeutic groups. Examples of suitable effector groups include calicheamicins, auristatins, geldanamycins, alpha-amanitine, pyrrolobenzodiazepines and maytansines.

The term “label” or “labelling group” refers to any detectable label. In general, labels fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g. horseradish peroxidase, β-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.).

In an alternative aspect, the invention also pertains to a endoglin binding molecule, comprising at least a heavy chain complementary determining region (CDR) 3 having the sequence RNYVTGFDY (SEQ ID NO: 16), or a sequence with no more than 3, 2, preferably no more than 1, amino acid mutations compared to this sequence; and a light chain CDR3 having the sequence HQYLSSYT (SEQ ID NO: 20), or a sequence with no more than 3, 2, preferably no more than 1, amino acid mutations compared to this sequence.

In one additional aspect the endoglin binding molecule is an ABP, and comprises at least one, preferably two, antibody heavy chain sequences, and at least one, preferably two, antibody light chain sequences, wherein the at least one, preferably two, antibody heavy chain sequences comprises an HCDR1 having the sequence SYWMH (SEQ ID NO:14), and an HCDR2 having the sequence NIYPGSGSTFYDEKFKG (SEQ ID NO:15), and an HCDR3 having the sequence RNYVTGFDY (SEQ ID NO:16); and/or wherein the at least one, preferably two, antibody light chain sequences comprises an LCDR1 having the sequence KSSQSVLYSSNQKNYLA (SEQ ID NO:18), and an LCDR2 having the sequence WASTRES (SEQ ID NO:19), and an LCDR3 having the sequence HQYLSSYT (SEQ ID NO:20). Optionally, wherein the each of the sequence have no more than three, preferably no more than two, more preferably no more than 1, amino acid variations compared to the indicated sequences—such as an amino acid addition, deletion, insertion, or substitution.

In a further embodiment of this aspect, the endoglin antibody may be a mono specific ABP, such as an IgG, or may be used in context of the other aspects of the herein disclosed invention as a “second binding site” of the bispecific molecule mediating a binding to a tumor associated antigen.

Preferably the endoglin binding molecule is an ABP, wherein the heavy chain sequence comprises an antibody heavy chain variable region shown in SEQ ID NO: 17; optionally, wherein the each of the sequence have no more than 10, 9, 8, 7, 6, 5, 4, 3, preferably no more than two, more preferably no more than 1, amino acid variations compared to the indicated sequences—such as an amino acid addition, deletion, insertion, or substitution; and/or wherein the light chain sequence comprises an antibody heavy chain variable region shown in SEQ ID NO: 21; optionally, wherein the each of the sequence have no more than 10, 9, 8, 7, 6, 5, 4, 3, preferably no more than two, more preferably no more than 1, amino acid variations compared to the indicated sequences—such as an amino acid addition, deletion, insertion, or substitution.

Further alternative aspects then pertain to nucleic acid sequences encoding the anti-endoglin ABP of the invention, vectors for the expression of the heavy chain and/or light chain sequences of the anti-endoglin ABP of the invention. Exemplary such encoding nucleic acid sequences are shown in SEQ ID NO: 22 and 23.

It is to be understood that the general descriptions herein above in context of antibodies for the sake of conciseness is not repeated but correspondingly applies.

In a second aspect, the invention pertains to an isolated nucleic acid comprising a sequence encoding for a binding molecule of the invention, or for an antigen binding fragment or a monomer, such as a heavy or light chain, of a binding molecule, of any one of the first aspect, or encoding for a bispecific ABP according to the invention.

For example, the component encoded by a nucleic acid of the invention may be all or part of one chain of an antibody of the invention; or the component may be a scFv of said binding molecule. The component encoded by such a nucleic acid may be all or part of one or other of the chains of an antibody of the invention; for example, the component encoded by such a nucleic acid may be a binding molecule of the invention. The nucleic acids of the invention may also encode a fragment, derivative, mutant, or variant of a binding molecule of the invention, and/or represent components that are polynucleotides suitable and/or sufficient for use as hybridisation probes, polymerase chain reaction (PCR) primers or sequencing primers for identifying, analyzing, mutating or amplifying a polynucleotide encoding a polypeptide, anti-sense or inhibitory nucleic acids (such as RNAi/siRNA/shRNA or gRNA molecules) for inhibiting expression of a polynucleotide, and complementary sequences of the foregoing.

In particular embodiments of the invention, a nucleic acid of the invention comprises a nucleic acid having a sequence encoding a heavy or light chain CDR, a combination of heavy and/or light chain CDR1, CDR2 and CDR3 or a heavy or light chain variable domain, in each case as displayed in a sequence of table 1, or a functional fragment thereof. In other embodiments, a nucleic acid of the invention comprises a nucleic acid sequence at least 60%, 65%, 70%, 75%, 80%, 85%, 90%; or 95% (preferably at least 75%) sequence identity to (or having no more than fifty, forty, thirty, twenty, fifteen, ten or five, preferably no more than three, two or one, base substitution(s), insertion(s) or deletion(s), to a sequence encoding any of the herein disclosed CDRs, preferably CDR3, in table 1.

The nucleic acid according to the invention may be a DNA or RNA of genomic, mRNA, cDNA, or synthetic origin or some combination thereof, optionally linked to a polynucleotide to which it is not linked in nature. In some embodiments, such nucleic acid may comprise one or more (such as 2, 3, 4, 5, 6, 7, 8, 9, 10 or more than 20, in particular between 1 and about 5, or preferably all instances of a particular nucleotide in the sequence) unnatural (e.g. synthetic) nucleotides; and/or such nucleic acid may comprise (e.g. is conjugated to) another chemical moiety, such as a labelling group or an effector group; for example, a labelling group or an effector group as described elsewhere herein.

In one embodiment, the nucleic acid of the invention may be isolated or substantially pure. In another embodiment, the nucleic acid of the invention may be recombinant, synthetic and/or modified, or in any other way non-natural. For example, a nucleic acid of the invention may contain at least one nucleic acid substitution (or deletion) modification (such as 2, 3, 4, 5, 6, 7, 8, 9, 10 or more than 10 such modifications, in particular between 1 and about 5 such modifications, preferably 2 or 3 such modifications) relative to a product of nature, such as a human nucleic acid.

The nucleic acids can be any suitable length, such as about 10, 15, 20, 25, 30, 35, 40, 45, 75, 100, 125, 150, 175, 200, 250, 300, 350, 400, 450, 500, 750, 1,000, 1,500, 3,000, 5,000 or more nucleotides in length. For example: siRNA nucleic acids may, preferably, be between about to about 25 base pairs in length (preferably between about 19 and about 21 base pairs in length); shRNA nucleic acids may, preferably, comprise a 20-30 base pair stem, a loop of at least 4 nucleotides, and a dinucleotide overhang at the 3′ end; microRNA may, preferably, be about 22 base pairs in length; an mRNA or DNA sequence encoding an ABP or a component thereof (such as a heavy or light chain or an IgG antibody) of the invention may, preferably, be between about 500 and 1,500 nucleotides. More preferably, a nucleic acid encoding a mammalian light chain of an antibody may be between about 630 and about 650 nucleotides, and one encoding a mammalian heavy chain of an antibody may be between about 1,300 and about 1,650 nucleotides. A nucleic acid can comprise one or more additional sequences, for example, regulatory sequences, and/or be part of a larger nucleic acid. The nucleic acids can be single-stranded or double-stranded and can comprise RNA and/or DNA nucleotides, and artificial variants thereof (e.g., peptide nucleic acids).

Changes can be introduced by mutation into the sequence of a nucleic acid of the invention. Such changes, depending on their nature and location in a codon, can lead to changes in the amino acid sequence of a polypeptide (e.g., an antigen binding protein) that it encodes. Mutations can be introduced using any technique known in the art.

In one embodiment, one or more particular amino acid residues may be changed using, for example, a site-directed mutagenesis protocol. In another embodiment, one or more randomly selected residues may be changed using, for example, a random mutagenesis protocol. However, it is made, a mutant polypeptide can be expressed and screened for a desired property. Mutations can be introduced into a nucleic acid without significantly altering the biological activity of a polypeptide that it encodes. For example, one can make nucleotide substitutions leading to amino acid substitutions at non-essential amino acid residues.

Other changes that may be made (e.g. by mutation) to the sequence of a nucleic acid of the invention may not alter the amino acid sequence of the encoded polypeptide, but may lead to changes to its stability and/or effectiveness of expression of the encoded polypeptide. For example, by codon optimization, the expression of a given polypeptide sequence may be improved by utilizing the more common codons for a given amino acid that are found for the species in which the nucleotide is to be expressed. Methods of codon optimization, and alternative methods (such as optimization of CpG and G/C content), are described in, for example, Hass et al, 1996 (Current Biology 6:315); WO1996/09378; WO2006/015789 and WO 2002/098443).

In a third aspect, the invention pertains to a nucleic acid construct (NAC) comprising a nucleic acid of the third aspect and one or more additional sequence features permitting the expression of the encoded binding molecule (or further binding molecule) P, or a component of said binding molecule or further binding molecule (such as an antibody heavy chain or light chain) in a cell.

Such an NAC can comprise one or more additional features permitting the expression of the encoded binding molecule or component of said binding molecule (eg the antigen binding site) in a cell (such as in a host cell). Examples of NACs of the invention include, but are not limited to, plasmid vectors, viral vectors, mRNA, non-episomal mammalian vectors and expression vectors, for example, recombinant expression vectors. The nucleic acid constructs of the invention can comprise a nucleic acid of the invention in a form suitable for expression of the nucleic acid in a cell, such as a host cell, (see below). The nucleic acid constructs of the invention will be, typically, recombinant nucleic acids, and/or may be isolated and/or substantially pure. Recombinant nucleic acids will, typically, be non-natural; particularly if they comprise portions that are derived from different species and/or synthetic, in-vitro or mutagenic methods.

In some embodiments, an NAC of the invention comprises one or more constructs either of which includes a nucleic acid encoding either a heavy or a light antibody chain. In some embodiments, the NAC of the invention comprises two constructs, one of which includes a nucleic acid encoding the heavy antibody chain, the other of which includes a nucleic acid encoding the light antibody chain, such that expression from both constructs can generate a complete antibody molecule. In some embodiments, the NAC of the invention comprises a construct which includes nucleic acids encoding both heavy and light antibody chains, such that a complete antibody molecule can be expressed from one construct. In other embodiments, an NAC of the invention can comprise a single construct that encodes a single chain which is sufficient to form an ABP of the invention; for example, if the encoded binding molecule is a scFv or a single-domain antibody (such as a camelid antibody).

In some embodiments, the NAC of the invention includes sequences encoding all or part of a constant region, enabling an entire, or a part of, a heavy and/or light chain to be expressed.

An NAC according to the invention may comprise (or consist of) a mRNA molecule which includes an open reading frame encoding a binding molecule of the invention, and for example together with upstream and downstream elements (such as 5′ and/or 3′ UTRs and/or poly-A stretch) that enables expression of the binding molecule, and preferably enhancing stability of the mRNA and/or expression of the binding molecule. The use of mRNA as NACs to introduce into and express polynucleotides in cells is described, for example, in Zangi et al in Nat. Biotechnol. vol. 31, 898-907 (2013), Sahin et al (2014) Nature Reviews Drug Discovery 13:759 and by Thess et al in Mol. Ther. vol. 23 no.9, 1456-1464 (2015). Particular UTRs that may be comprised in an mRNA NAC of the invention include: 5′UTR of a TOP gene (WO2013/143699), and/or a histone stem-loop (WO 2013/120629). An mRNA NAC of the invention may further comprise one or more chemical modifications (EP 1 685 844); including a 5′-cap, such as m7G(5′)ppp, (5′(A,G(5′)ppp(5′)A or G(5′)ppp(5′)G and/or at least one nucleotide that is an analogue of naturally occurring nucleotides, such as phosphorothioates, phosphoroamidates, peptide nucleotides, methylphosphonates, 7-deaza-guanosine, 5-methylcytosine or inosine.

NACs, such as DNA-, retroviral- and mRNA-based NACs of the invention may be used in genetic therapeutic methods in order to treat or prevent diseases of the immune system (see Methods of Treatment below), whereby an NAC that comprises an expressible sequence encoding an ABP of the invention is administered to the cell or organism (e.g. by transfection). In particular, the use of mRNA therapeutics for the expression of antibodies is known from WO2008/083949.

Preferably, in context of the second and third aspect, the nucleic acid may comprise a sequence encoding for a protein having an amino acid sequence of any one of SEQ ID NO: 1 to 12 of table 1.

In a fourth aspect, the invention pertains to a recombinant host cell comprising a nucleic acid or a NAC according to the third or fourth aspect. Preferably, such cell is capable of expressing the binding molecule (or component thereof) encoded by said NAC(s). For example, if a binding molecule of the invention comprises two separate polypeptide chains (e.g. a heavy and light chain of an IgG), then the cell of the invention may comprise a first NAC that encodes (and can express) the heavy chain of such binding molecule as well as a second NAC that encodes (and can express) the light chain of such binding molecule; alternatively, the cell may comprise a single NAC that encodes both chains of such binding molecule. In these ways, such a cell of the invention would be capable of expressing a functional (e.g. binding and/or inhibitory) binding molecule of the invention. A (host) cell of invention may be one of the mammalian, prokaryotic or eukaryotic host cells as described elsewhere herein, in particularly where the cell is a Chinese hamster ovary (CHO) cell.

In certain embodiments of such aspect, the (host) cell is a human cell; in particular it may be a human cell that has been sampled from a specific individual (eg an autologous human cell). In such embodiments, such human cell can be propagated and/or manipulated in-vitro so as to introduce a NAC of the present invention. The utility of a manipulated human cell from a specific individual can be to produce a binding molecule of the invention, including to reintroduce a population of such manipulated human cells into a human subject, such as for use in therapy. In certain of such uses, the manipulated human cell may be introduced into the same human individual from which it was first sampled; for example, as an autologous human cell.

The human cell that is subject to such manipulation can be of any germ cell or somatic cell type in the body. For example, the donor cell can be a germ cell or a somatic cell selected from the group consisting of fibroblasts, B cells, T cells, dendritic cells, keratinocytes, adipose cells, epithelial cells, epidermal cells, chondrocytes, cumulus cells, neural cells, glial cells, astrocytes, cardiac cells, oesophageal cells, muscle cells, melanocytes, hematopoietic cells, macrophages, monocytes, and mononuclear cells. The donor cell can be obtained from any organ or tissue in the body; for example, it can be a cell from an organ selected from the group consisting of liver, stomach, intestines, lung, pancreas, cornea, skin, gallbladder, ovary, testes, kidneys, heart, bladder, and urethra.

In a fifth aspect, the invention pertains to a pharmaceutical composition comprising: (i) binding molecule of the first aspect, or (ii) a nucleic acid or NAC of the second or third aspect, or (iii) a recombinant host cell according to the fourth aspect, and a pharmaceutically acceptable carrier, stabiliser and/or excipient.

In a sixth aspect, the invention pertains to a kit of packages or pharmaceutical compositions, the kit comprising in separate containers: (i) an isolated binding molecule recited in any one of the preceding aspects, an isolated nucleic acid encoding the isolated binding molecule, and/or a recombinant host cell comprising such nucleic acid or isolated binding molecule; and (ii) an isolated further binding molecule recited in any one of the preceding aspects, an isolated nucleic acid encoding the isolated further binding molecule, and/or a recombinant host cell comprising such nucleic acid or isolated further binding molecule.

To be used in therapy, the binding molecules, nucleic acids or NACs (or the cells, such as host cells) of the invention may be formulated into a pharmaceutical composition appropriate to facilitate administration to animals or humans. The term “pharmaceutical composition” means a mixture of substances including a therapeutically active substance (such as an ABP of the invention) for pharmaceutical use.

By way of example, the pharmaceutical composition of the invention may comprise between 0.1% and 100% (w/w) active ingredient, such as about 0.5%, 1%, 2%, 3%, 4%, 5%, 6%, 8% 10%, 15%, 20%, 25%, 30%, 40%, 50%, 60%, 70%, 80%, 90%, 95%, 96%, 97%, 98% or 99%, preferably between about 1% and about 20%, between about 10% and 50% or between about 40% and 90%.

As used herein the language “pharmaceutically acceptable” excipient, stabiliser or carrier is intended to include any and all solvents, solubilisers, fillers, stabilisers, binders, absorbents, bases, buffering agents, lubricants, controlled release vehicles, diluents, emulsifying agents, humectants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

The pharmaceutical composition of (or for use with) the invention is, typically, formulated to be compatible with its intended route of administration. Examples of routes of administration include oral, parenteral, e.g., intrathecal, intra-arterial, intravenous, intradermal, subcutaneous, oral, transdermal (topical) and transmucosal administration.

In some embodiments, the pharmaceutical composition comprising a binding molecule or NAC is in unit dose form of between 10 and 1000 mg. In some embodiments, the pharmaceutical composition comprising a binding molecule or NAC is in unit dose form of between 10 and 200 mg. In some embodiments, the pharmaceutical composition comprising a binding molecule is in unit dose form of between 200 and 400 mg. In some embodiments, the pharmaceutical composition comprising a binding molecule or NAC is in unit dose form of between 400 and 600 mg. In some embodiments, the pharmaceutical composition comprising a binding molecule or NAC is in unit dose form of between 600 and 800 mg. In some embodiments, the pharmaceutical composition comprising a binding molecule or NAC is in unit dose form of between 800 and 100 mg.

Exemplary unit dosage forms for pharmaceutical compositions comprising a binding molecule or NAC are tablets, capsules (eg as powder, granules, microtablets or micropellets), suspensions or as single-use pre-loaded syringes. In certain embodiments, kits are provided for producing a single-dose administration unit. The kit can contain both a first container having a dried active ingredient and a second container having an aqueous formulation. Alternatively, the kit can contain single and multi-chambered pre-loaded syringes.

Toxicity and therapeutic efficacy (eg effectiveness) of such active ingredients can be determined by standard pharmaceutical procedures in cell cultures or experimental animals, eg, for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Active agents which exhibit large therapeutic indices are preferred. While compounds that exhibit toxic side effects maybe used, care should be taken to design a delivery system that targets such compounds to the site of affected tissue in order to minimise potential damage to uninfected cells and, thereby, reduce side effects.

In accordance with all aspects and embodiments of the medical uses and methods of treatment provided herein, the effective amount administered at least once to a subject in need of treatment with a binding molecule or NAC is, typically, between about 0.01 mg/kg and about 100 mg/kg per administration, such as between about 1 mg/kg and about 10 mg/kg per administration. In some embodiments, the effective amount administered at least once to said subject of a ABP or NAC is between about 0.01 mg/kg and about 0.1 mg/kg per administration, between about 0.1 mg/kg and about 1 mg/kg per administration, between about 1 mg/kg and about 5 mg/kg per administration, between about 5 mg/kg and about 10 mg/kg per administration, between about 10 mg/kg and about 50 mg/kg per administration, or between about 50 mg/kg and about 100 mg/kg per administration.

For the prevention or treatment of disease, the appropriate dosage of a binding molecule or NAC (or a pharmaceutical composition comprised thereof) will depend on the type of disease to be treated, the severity and course of the disease, whether the binding molecule or NAC and/or pharmaceutical composition is administered for preventive or therapeutic purposes, previous therapy, the patient's clinical history, age, size/weight and response to a binding molecule or NAC and/or pharmaceutical composition, and the discretion of the attending physician. The binding molecule or NAC and/or pharmaceutical composition is suitably administered to the patient at one time or over a series of treatments. If such binding molecule or NAC and/or pharmaceutical composition is administered over a series of treatments, the total number of administrations for a given course of treatment may consist of a total of about 2, 3, 4, 5, 6, 7, 8, 9, 10 or more than about 10 treatments. For example, a treatment may be given once every day (or 2, 3 or 4 times a day) for a week, a month or even several months. In certain embodiments, the course of treatment may continue indefinitely.

In a sixth aspect, the invention pertains to a component for use in medicine, wherein the component is selected from the list consisting of: (i) a binding molecule or bispecific ABP of the first aspect, or (ii) a nucleic acid or NAC of the second or third aspect, or (iii) a recombinant host cell according to the fourth aspect and (iv) a pharmaceutical composition or kit according to the fifth aspect.

In a related aspect, the invention also relates to method of treating or preventing a disease, disorder or condition in a mammalian subject in need thereof, comprising administering to said subject at least once an effective amount of modulating compound as desired above, or, and in particular administering to said subject at least once an effective amount of the binding molecule, the NAC, the (host) cells, or the pharmaceutical composition as described above.

In another related aspect, the invention also relates to the use of a product of the invention as describe above, or a modulating compound as described above (in particular a binding molecule of the invention) for the manufacture of a medicament, in particular for the treatment of a disease, disorder or condition in a mammalian subject, in particular where the disease, disorder or condition is one as set out herein.

The term “treatment” in the present invention is meant to include therapy, e.g. therapeutic treatment, as well as prophylactic or suppressive measures for a disease (or disorder or condition). Thus, for example, successful administration of a compound according to the invention prior to onset of the disease results in treatment of the disease. “Treatment” also encompasses administration of a compound of the invention after the appearance of the disease in order to ameliorate or eradicate the disease (or symptoms thereof). Administration of a CD28 binding molecule of the invention after onset and after clinical symptoms, with possible abatement of clinical symptoms and perhaps amelioration of the disease, also comprises treatment of the disease. Those “in need of treatment” include subjects (such as a human subject) already having the disease, disorder or condition, as well as those prone to or suspected of having the disease, disorder or condition, including those in which the disease, disorder or condition is to be prevented.

In particular embodiments of these aspects, the modulating compound is one described above, and/or is a binding molecule, NAC, a (host) cell, or a pharmaceutical composition or kit of the present invention; in particular is a binding molecule of the invention.

In other aspects described elsewhere herein, are provided methods to detect and/or diagnose a disease, disorder or condition in a mammalian subject.

In one particular embodiment, the disease, disorder or condition that is characterized by a pathological immune response.

In another particular embodiment, the disease, disorder or condition is a proliferative disorder (or a condition associated with such disorder or disease), in particular when the product or modulating compound (such as a binding molecule, nucleic acid, NAC or recombinant host cell of the invention, in particular a binding molecule of the invention).

A “proliferative disorder” refers to a disorder characterized by abnormal proliferation of cells. A proliferative disorder does not imply any limitation with respect to the rate of cell growth, but merely indicates loss of normal controls that affect growth and cell division. Thus, in some embodiments, cells of a proliferative disorder can have the same cell division rates as normal cells but do not respond to signals that limit such growth. Within the ambit of “proliferative disorder” is neoplasm or tumor, which is an abnormal growth of tissue or cells. Cancer is art understood, and includes any of various malignant neoplasms characterized by the proliferation of cells that have the capability to invade surrounding tissue and/or metastasise to new colonisation sites. Proliferative disorders include cancer, atherosclerosis, rheumatoid arthritis, idiopathic pulmonary fibrosis and cirrhosis of the liver. Non-cancerous proliferative disorders also include hyperproliferation of cells in the skin such as psoriasis and its varied clinical forms, Reiter's syndrome, pityriasis rubra pilaris, and hyperproliferative variants of disorders of keratinization (e.g., actinic keratosis, senile keratosis), scleroderma, and the like.

In more particular embodiments, the proliferative disorder is a cancer or tumor, in particular a solid tumor (or a condition associated with such cancer or tumor). Such proliferative disorders include, but are not limited to, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, any haemotological malignancy (e.g., chronic lymphoblastic leukemia, chronic myelomonocytic leukemia, acute lymphoblastic leukemia, acute lymphocytic leukemia, acute myelogenous leukemia, acute myeloblasts leukemia, chronic myeloblastic leukemia, Hodgkin's disease, non- Hodgkin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, marginal zone lymphoma, Burkitt Lymphoma, mycosis fungoides, seary syndrome, cutaneous T-cell lymphoma, peripheral T cell lymphoma, chronic myeloproliferative disorders, myelofibrosis, myeloid metaplasia, systemic mastocytosis), and cental nervous system tumors (e.g., brain cancer, glioblastoma, non- glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma), myeloproliferative disorders (e.g., polycythemia vera, thrombocythemia, idiopathic myelofibrosis), soft tissue sarcoma, thyroid cancer, endometrial cancer, carcinoid cancer, or liver cancer.

In one preferred embodiment, the various aspects of the invention relate to, for example a binding molecule of the invention used to detect/diagnose, prevent and/or treat, such proliferative disorders that include but are not limited to carcinoma (including breast cancer, prostate cancer, gastric cancer, lung cancer, colorectal and/or colon cancer, hepatocellular carcinoma, melanoma), lymphoma (including non-Hodgkin's lymphoma and mycosis fungoides), leukemia, sarcoma, mesothelioma, brain cancer (including glioma), germinoma (including testicular cancer and ovarian cancer), choriocarcinoma, renal cancer, pancreatic cancer, thyroid cancer, head and neck cancer, endometrial cancer, cervical cancer, bladder cancer, or stomach cancer.

Accordingly, in a preferred embodiment, a binding molecule, or NAC according to the invention, is for use in the prevention and/or treatment of a cancer, for example a cancer which is characterized by the presence of a cancer cell selected from the group consisting of a cell of an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a phaeochromocytoma, a pituitary tumor, a prostate cancer, a posterious unveal melanoma, a rare hematologic disorder, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

In other methods, the modulating (eg inhibiting) compound (eg a binding molecule, such as one of the present invention) may be used is in combination with a different anti-proliferative therapy, in particular a different anti-cancer therapy, in particular where the different anti-proliferative therapy is immunotherapy, in particular immunotherapy with a ligand to an immune checkpoint molecule. Accordingly, the composition can be for use in the treatment of a proliferative disorder in a subject in need thereof, where the subject is subjected to co-treatment by immunotherapy, in particular co-therapy (eg combination treatment) with a ligand to an immune checkpoint molecule.

In such embodiments, the ligand is one that binds to an immune (inhibitory) checkpoint molecule. For example, such checkpoint molecule may be one selected from the group consisting of: A2AR, B7-H3, B7-H4, CTLA-4, IDO, KIR, LAGS, PD-1 (or one of its ligands PD-L1 and PD-L2), TIM-3 (or its ligand galectin-9), TIGIT, or for example antigen binding molecules targeting FLT3, PSMA or other tumor associated targets. In particular of such embodiments, the ligand binds to a checkpoint molecule selected from: CTLA-4, PD-1 and PD-L1. In other more particular embodiments, the ligand is an antibody selected from the group consisting of: ipilimumab, nivolumab, pembrolizumab, BGB-A317, atezolizumab, avelumab and durvaluma; in particular an antibody selected from the group consisting of: ipilimumab (YERVOY), nivolumab (OPDIVO), pembrolizumab (KEYTRUDA) and atezolizumab (TECENTRIQ).

When a method or use in therapy of the present invention (eg, one involving an ABP of the invention) is used in combination treatments together with any of such other procedures (eg, another agent or a cancer immunotherapy, such as a ligand that binds to an immune (inhibitory) checkpoint molecule), then such method or use being a combination treatment regimen may comprise embodiments where such exposures/administrations are concomitant. In alternative embodiments, such administrations may be sequential; in particular those embodiments where an ABP of the invention is administered before such other procedure. For example, such compound of the invention may be sequentially administered within about 14 days of (eg before) the other procedure, such as within about 10 days, 7 days, 5 days, 2 days or 1 day of (eg before) is the other procedure; and further including where the compound of the invention may be sequentially administered within about 48 hours, 24 hours, 12 hours, 8 hours, 6 hours, 4 hours, 2 hours, 1 hours, 30 mins, 15 mins or 5 mins of (eg before) the other procedure.

In a further aspect the invention pertains to a method for the prevention and/or treatment of a proliferative disorder in a subject, comprising the administration of a therapeutically effective amount of a component recited in the present disclosure to a subject in need of the treatment; and wherein the proliferative disorder is characterized by an expression of an antigenic protein in cells associated with the proliferative disorder. In preferred embodiments, and as disclosed herein elsewhere, such treatment comprises an additional administration of a further binding molecule.

As described above, in one aspect, herein provided is a cell, such as (recombinant) host cell or a hybridoma capable of expressing an ABP as described above. In an alternative aspect, herein provided is a cell, which comprises at least one NAC encoding an ABP or a component of an ABP as described above. Cells of the invention can be used in methods provided herein to produce the ABPs and/or NACs of the invention.

Accordingly, in another aspect the invention relates to a method of producing a

recombinant cell line capable of expressing a binding molecule of the first aspect, the method comprising the steps of:
providing a suitable host cell;
providing at least one genetic construct comprising coding sequence(s) encoding the binding molecule of the present invention;
introducing into said suitable host cell said genetic construct(s); and
optionally, expressing said genetic construct(s) by said suitable host cell under conditions that allow for the expression of the binding molecule.

In yet another aspect, herein provided is a method of producing a binding molecule as described above, for example comprising culturing one or more cells of the invention under conditions allowing the expression of said binding molecule.

In some further aspect and embodiments, the present invention pertains to the following items which shall be interpreted considering the other herein disclosed and described aspects and embodiments, as well as the examples:

Item 1. A binding molecule which is at least bispecific comprising at least two first antigen binding sites and at least one second antigen binding site for use in the treatment of a disease in a subject, wherein
(i)The at least two first antigen binding sites are capable of specifically binding to an epitope of T-cell-specific surface glycoprotein CD28; and
(ii)The at least one second antigen binding site is capable of binding to an epitope of an antigenic target protein expressed on or in a cell associated with the disease in the subject;
   wherein the treatment comprises an administration of the binding molecule to the subject.

Item 2. The binding molecule for use of item 1, wherein the at least two first and/or the at least one second antigen binding site(s) are derived from an antibody or antibody like molecule, and wherein the at least two first antigen binding sites are each provided as an antigen binding fragment of an antibody which is not a F(ab′)₂ or Fab, and preferably is a single-chain construct, most preferably as a single chain Fv (scFv).

Item 3. The binding molecule for use of item 1 or 2, wherein the binding molecule when contacted with a first cell that is a CD28 positive immune cell (such as a T-cell) in absence of a second cell expressing the antigenic target protein, does not induce CD28 signalling and preferably does not activate the immune cell (T cell).

Item 4. The binding molecule for use of any one of items 1 to 3, wherein the antigenic target protein is selected from a protein expressed on cells associated with a proliferative disorder, a protein or other molecule associated with a pathogenic organism, such as a parasite, virus or a bacterium.

Item 5. The binding molecule for use of any one of items 1 to 4, wherein the antigenic target protein is endoglin.

Item 6. The binding molecule for use of any one of items 1 to 5, wherein each one of the at least two first antigen binding sites is directly, such as covalently or non-covalently, connected with the at least one second antigen binding site.

Item 7. The binding molecule for use of any one of items 1 to 6, comprising at least two second antigen binding sites.

Item 8. The binding molecule for use of any one of items 1 to 7, wherein the at least two first antigen binding sites bind the same epitope on CD28, preferably wherein the at least two antigen binding sites are identical.

Item 9. The binding molecule for use of any one of items 1 to 8, wherein at least one of the at least two first antigen binding sites and the at least one second antigen binding site are linked to each other by a protein-linker comprising one or more antibody-derived human constant domains, preferably of an IgG, for example they are linked via human IgG-derived CH1, CH₂ and/or CH_{3 .}

Item 10. The binding molecule for use of any one of items 1 to 9, wherein the at least two first antigen binding sites comprise an antibody heavy chain sequence and an antibody light chain sequence (preferably at least CDR1 to CDR3), each derived from, and competitively binding to the same antigen as, the C-terminal binding site comprised in an antibody sequence shown in SEQ ID NO: 2, 3, 4, 5, 7, 8 or 9.

Item 11. The binding molecule for use of any one of items 1 to 10, wherein the at least one second antigen binding site comprises an antibody heavy chain sequence and an antibody light chain sequence, each derived from, and competitively binding to the same antigen as, the N-terminal binding site comprised in an antibody composed of SEQ ID NO: 1 and 2.

Item 12. The binding molecule for use of any one of items 1 to 11, wherein the binding molecule specifically binds to an immune cell and a cell associated with the disease, preferably wherein the immune cell is an immune cell involved with a cell-mediated immune response, such as cytotoxic immune response or a helper cell mediated immune response.

Item 13. The binding molecule for use of any one of items 1 to 12, wherein the immune cell is preferably a cytotoxic cell or helper cell, such as a cell expressing CD28 and CD3 (and a TCR) and preferably is a T cell.

Item 14. The binding molecule for use of any one of items 1 to 13, wherein the subject is characterized in that CD3/TCR signalling is activated by treatment, or endogenously in response to a disease-associated antigen, such as an antigenic protein.

Item 15. The binding molecule for use of any one of items 1 to 14, wherein the treatment further comprises stimulation and/or activation of immune cells towards cells associated with the disease, such as an activation and/or stimulation of T-cells.

Item 16. The binding molecule for use of any one of items 1 to 15, comprising two antibody heavy chain sequences, and two antibody light chain sequences, and wherein
(i) One of the at least two first antigen binding sites is covalently connected to a C-terminal end of one of the two antibody light chain sequences, and the other of the at least two first antigen binding sites is covalently connected to a C-terminal end of the other of the two antibody light chain sequences; or
(ii) One of the at least two first antigen binding sites is covalently connected to a C-terminal end of one of the two antibody heavy chain sequences, and the other of the at least two first antigen binding sites is covalently connected to a C-terminal end of the other of the two antibody heavy chain sequences.

Item 17. The binding molecule for use of item 16, wherein the binding sites are connected either without a peptide linker or with a short peptide linker having not more than amino acids, or via a long peptide linker having at least 6, preferably up to 50 amino acids, wherein further preferably the peptide linker is a (GGGGS)ₙ linker, and n is larger or equal to two.

Item 18. The binding molecule for use of any one of the preceding items, wherein the at least one second antigen binding site, comprises a Fab, F(ab′)₂ or most preferably an IgG.

Item 19. The binding molecule for use of item any one of items 1 to 18, wherein the treatment comprises the sequential or concomitant administration of (i) a further binding molecule which is bispecific and which is capable of specifically binding to (and activating) a T cell, such as via binding to CD3 and/or a T cell receptor (TCR); or any other reagent capable of providing or enhancing signals for T cell activation such as (ii) genetically modified immune cells (heterologous or autologous T-cell) expressing an antigen receptor, such as a chimeric antigen receptor (CAR), which receptor is capable of specifically binding the antigenic target protein or (iii) vaccines providing antigenic structures from infectious agents or cancer cells (TAA or TSA) or (iv) reagents that block suppressive “second signals”, via checkpoint molecules such as PD1. Such reagents are termed checkpoint blockers.

Item 20. The binding molecule for use of item 19, wherein the further binding molecule is at least bispecific and comprises at least one third antigen binding site and at least one fourth antigen binding site, wherein
(i) The least one third antigen binding site is capable of specifically binding to CD3 and/or a T cell receptor (TCR) (CD3/TCR); and
(ii) The at least one fourth antigen binding site is capable of specifically binding to an epitope of a further antigenic target protein expressed on or in a cell associated with the disease in the subject.

Item 21. The binding molecule for use of item 17, wherein the antigenic target protein and the further antigenic target protein are (i) identical or (ii) different but in close spatial proximity to each other, such as being expressed on the same cell associated with the disease, or located in the same diseases tissue such as being expressed in the same tumor environment.

Item 22. The binding molecule for use of any one of items 1 to 18, wherein the disease is a proliferative disease, preferably selected from a cancer disease, such as a cancer, for example lung cancer, breast cancer, colorectal cancer, gastric cancer, hepatocellular carcinoma, pancreatic cancer, ovarian cancer, melanoma, myeloma, kidney cancer, head and neck cancer, Hodgkin lymphoma, bladder cancer or prostate cancer, in particular one selected from the list consisting of: melanoma, lung cancer (such as non-small cell lung cancer), bladder cancer (such as urothelial carcinoma), kidney cancer (such as renal cell carcinoma), head and neck cancer (such as squamous cell cancer of the head and neck) and Hodgkin lymphoma. Preferably, the proliferative disease is melanoma, or lung cancer (such as non-small cell lung cancer), preferably a cancer positive for an expression of the target antigenic protein.

Item 23. An isolated binding molecule, wherein the isolated binding molecule is the binding molecule as recited in any one of the preceding items.

Item 24. An isolated nucleic acid encoding for an isolated binding molecule of item 23.

Item 25. A recombinant host cell, comprising an isolated binding molecule of item 23 or an isolated nucleic acid according to item 24.

Item 26. A pharmaceutical composition comprising an isolated binding molecule of item 23, an isolated nucleic acid of item 24, a recombinant host cell of item 25, together with a pharmaceutically acceptable carrier and/or excipient.

Item 27. The pharmaceutical composition of item 26, further comprising an isolated further binding molecule as recited in item 19 or 20.

Item 28. A kit of packages or pharmaceutical compositions, the kit comprising in separate containers: (i) an isolated binding molecule recited in any one of the preceding items, an isolated nucleic acid encoding the isolated binding molecule, and/or a recombinant host cell comprising such nucleic acid or isolated binding molecule; and (ii) an isolated further binding molecule recited in any one of the preceding items, an isolated nucleic acid encoding the isolated further binding molecule, and/or a recombinant host cell comprising such nucleic acid or isolated further binding molecule.

The terms “of the [present] invention”, “in accordance with the invention”, “according to the invention” and the like, as used herein are intended to refer to all aspects and embodiments of the invention described and/or itemed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES AND SEQUENCES

The figures show:

FIG. 1: shows a quantification of B7H3—and endoglin expression on different cell lines. The indicated cells, U937, HT-29 and HEK293T (DSMZ, Braunschweig, Germany) were incubated with the endoglin antibody mKro-22 and analyzed by flow cytometry using the QIFIKIT assay system (ND: not detectable).

FIG. 2: shows a selection of a high affinity binder from a panel of newly developed endoglin antibodies The affinity of the different antibodies was determined by flow cytometry (A) and Biacore analysis (B).

FIG. 3: shows Bispecific antibody variants used in this application: (A) A bsAb in the IgGsc format with B7H3xTCR/CD3—specificity (bsAbCD3) that provides an attenuated first signal for T cell activation; (B-E) Variants of bispecific endoglinxCD28 antibodies in the IgGsc format (BiCos). Single chain moieties were fused to the heavy and light chains of IgG antibodies as depicted using linkers of different lengths. The BiCo variants were designated Hc−L (B), Hc+L (C), Lc−L (D) and Lc+L (E); (F) A BiCo variant that contains its CD28 binding part as an N-terminal Fab2- rather than a C-terminal single chain moiety. See list of sequences for further information.

FIG. 4: shows a binding of the different BiCo variants to CD28 expressed on human CD4+ and CD8+ T cells. Binding of the variants to normal human T cells was determined after incubation of PBMC with the respective constructs and subsequent analysis by flow cytometry

FIG. 5: shows costimulatory activity of the BiCo variants on U937 and HT-29 target cells exhibiting high and low endoglin expression, respectively. Monocyte depleted PBMC were incubated with irradiated target cells as well as a bispecific B7H3xCD3 construct and one of four different BiCo variants at the indicated concentrations depicted at the upper left of (A) and (F), and at the X-axis, respectively. After 3 days of incubation 3H thymidine uptake was measured to assess T cell proliferation. In E and J control experiments in the absence of the respective target cells are shown. Concentrations of bsAbCD3 and BiCos in these experiments were 1 μg/ml and 2 μg/ml, respectively.

FIG. 6: shows costimulatory activity of the BiCo variants on U937 and HEK293 target cells with high and undetectable endoglin expression, respectively. Experimental conditions were identical to those in FIG. 5 except that fixed concentrations of the bsAbCD3 and the BiCos were used (0,2 μg/ml and 0,4 μg/ml, respectively).

FIG. 7: shows costimulatory activity of the BiCo variants and of the monospecific, bivalent CD28 antibody used for their construction. Experimental conditions were identical to those in FIG. 5. Antibody concentrations were 0.2 μg/ml for the bsAbCD3 and 1.0 μg/ml for all other antibodies.

FIG. 8: shows Enhancement of the antitumor activity of bsAbCD3 bei Bico1 (Lc+L variant) in an immunocompromised mouse model. 3x10exp6 HT-29 colon carcinoma cells were injected i.v. into NSG mice. 6 hours later, human PBMC (2x10exp 7) were intravenously applied alone or with 2nM bsAbCD3, 50nM BiCo-1 or 50nM BiCo-ctr, alone or in combination as indicated. Treatment with antibodies was repeated on day 4. At day 8, mice were sacrificed and human PBMCs and tumor cells in the lungs were quantified by flow cytometry after enzymatic digestion. Left panel, % of cancer cells in the lungs; right panel, effector cell proliferation. Statistical analysis was performed using Mann Whitney test (**p<0.01, ***p<0.001). BiCo-ctr is a BiCo with an unrelated target specificity (PSMAxCD28) in the LC+L format.

FIG. 9: shows the result of a further bispecific antibody of the invention (“BiCo-2”); the panel A depicts the architecture of both bispecific antibodies of the invention; the panel B depicts T cell proliferation by various concentrations of a BiCo with B7H3xCD28 specificity (BiCo-2, x-axis) in combination with various concentrations of CC-1 a bispecific antibody with PSMAxCD3-specificity (A left side). The right panel of the figure presents data using an identical experimental setting in which BiCo-2 is replaced by a control BiCo in which the PSMA-binding part is replaced by an antibody directed to an unrelated target antigen (MOPC). One representative of three experiments with peripheral blood mononuclear cells (PBMC) of different healthy donors is shown.

FIG. 10: shows the result of a further bispecific antibody of the invention (“BiCo-3”); the panel A depicts schematically the architecture of both bispecific antibodies of the invention; the panel B of the figure depicts T cell proliferation induced by a fixed concentration of two BiCo variants with FAPxCD28-specificity (BiCo-3, 0.1 μg/ml=0.5 nM) and a low concentration of a bispecific antibody with PSMAxCD3-specificity (CC-1, 1 ng/ml=0.005 nM) in the presence of either (i) two different target cells that express human FAP (hFAP transfected Sp2/0 cells) or PSMA (LNCaP cells), a setting that is designated as ,,Sp2/0hFAP″ or (ii) PSMA expressing cells and coated, recombinant FAP proteins (,,hFAP″). Bars designated ,,mFAP″ depict results in the presence of coated murine FAP protein. Note that two BiCo variants were used, one comprising an antibody exerting exclusive reactivity with human FAP (,,Sibrox9.3-8″) and one comprising an antibody exerting reactivity against human and murine FAP (,,MFP5x9.3-8″). In the panel C of the figure various control settings in the absence of antibodies are explored including those with the pan-clonal T cell stimulator PHA.

FIG. 11: shows a comparison of bispecific costimulatory antibody formats. The panel A depicts the constructs used in these experiments; panel B shows T cell proliferation in the presence of LNCaP cells expressing B7H3 (but not endoglin), a bispecific antibody (bsAb) with B7H3xCD3-specificity (CC-3) and various bsAb with endoglinxCD28 specificity. EngxCD28 Lc+L refers to the BiCo molecule that was used in the experiments depicted in, for example, FIG. 8 and designated BiCo-1. Common to these molecules is that the CD28 antibody is contained as a c-terminal bivalent single chain moiety. CD28xEngLc+L and CD28xEngHc+L refer to molecules with an inverted arrangement that is, an endoglin binding single c-terminal bivalent single chain moiety either fused to the light or the heavy chain of the n-terminal, bivalent CD28 antibody (BiCo-1 inverted in panel A). In the panel C shows various control settings in the absence of antibodies that were evaluated including one comprising the polyclonal T cell mitogen PHA.

FIG. 12: shows the sequence and indicated locations of mutations of an improved hu9.3.8.V1 CD28 antibody construct of the invention.

FIG. 13: shows the optimization of the BiCo construct of the invention. A sequence variant of the CD28 scFv was tested for protein aggregation. Characterization of BiCo-10pt (=variant 1), which is an improved version of the original BiCo-1 molecule that contains (i) a new Fc modification that provides attenuation of FcR binding as well as an elongation of serum half life (LALA-YTE) and (ii) 5 modifications in the VH and VL domains of the CD28 antibody to improve aggregation tendency and producibility.

FIG. 14: shows the comparison of the costimulatory activity of the old BiCo1-molecule and the optimized variant 1.

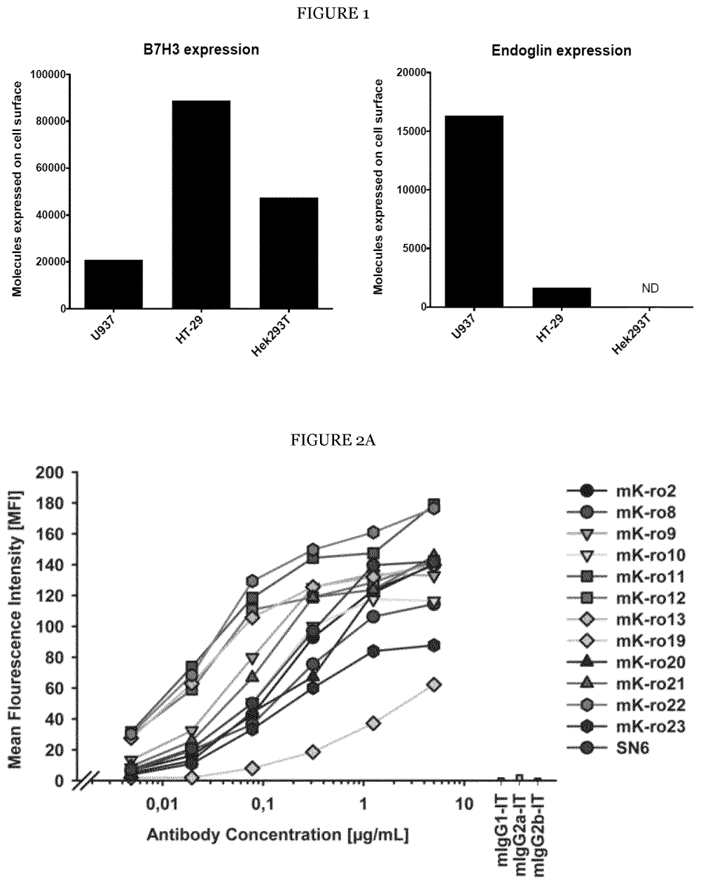

The sequences show:

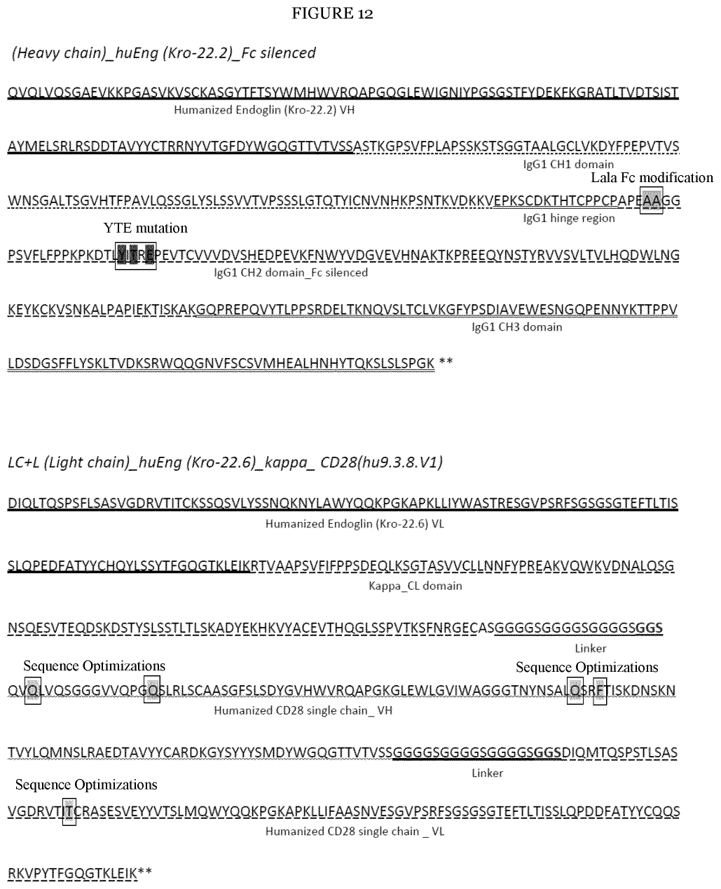

TABLE 1
Antibody Sequences of the Invention:

| SEQ ID | | |
|---|---|---|
| NO: | Description | Sequence |
| 1 | Heavy chain_hu | QVQLVQSGAEVKKPGASVKVSCKASGYTFTSYWMH |
| | Endoglin (K-r022- | WVRQAPGQGLEWIGNIYPGSGSTFYDEKFKGRATLT |
| | 2)_FcKO | VDTSISTAYMELSRLRSDDTAVYYCTRRNYVTGFDY |
| | | WGQGTTVTVSSASTKGPSVFPLAPSSKSTSGGTAALG |
| | | CLVKDYFPEPVTVSWNSGALTSGVHTFPAVLQSSGL |
| | | YSLSSVVTVPSSSLGTQTYICNVNHKPSNTKVDKKVE |
| | | PKSCDKTHTCPPCPAPPVAGPSVFLFPPKPKDTLMIS |
| | | RTPEVTCVVVGVSHEDPEVKFNWYVDGVEVHNAKT |
| | | KPREEQYNSTYRVVSVLTVLHQDWLNGKEYKCKVS |
| | | NKQLPSPIEKTISKAKGQPREPQVYTLPPSRDELTKN |
| | | QVSLTCLVKGFYPSDIAVEWESNGQPENNYKTTPPV |
| | | LDSDGSFFLYSKLTVDKSRWQQGNVFSCSVMHEAL |
| | | HNHYTQKSLSLSPGK |
| 2 | Light chain (Lc- | DIQLTQSPSFLSASVGDRVTITCKSSQSVLYSSNQKNY |
| | L)_hu Endoglin (K- | LAWYQQKPGKAPKLLIYWASTRESGVPSRFSGSGSG |
| | ro22-6)_kappa | TEFTLTISSLQPEDFATYYCHQYLSSYTFGQGTKLEIK |
| | huCD28 (9.3-8_Vh- | RTVAAPSVFIFPPSDEQLKSGTASVVCLLNNFYPREA |
| | Vl) | KVQWKVDNALQSGNSQESVTEQDSKDSTYSLSSTLT |
| | | LSKADYEKHKVYACEVTHQGLSSPVTKSFNRGECAS |
| | | QVQLVESGGGVVQPGRSLRLSCAASGFSLSDYGVHW |
| | | VRQAPGKGLEWLGVIWAGGGTNYNSALMSRKTISK |
| | | DNSKNTVYLQMNSLRAEDTAVYYCARDKGYSYYYS |
| | | MDYWGQGTTVTVSSGGGGSGGGGSGGGGSDIQMT |
| | | QSPSTLSASVGDRVTIICRASESVEYYVTSLMQWYQQ |
| | | KPGKAPKLLIFAASNVESGVPSRFSGSGSGTEFTLTIS |
| | | SLQPDDFATYYCQQSRKVPYTFGQGTKLEIKR |
| 3 | Light chain | DIQLTQSPSFLSASVGDRVTITCKSSQSVLYSSNQKNY |
| | (Lc + L)_hu Endoglin | LAWYQQKPGKAPKLLIYWASTRESGVPSRFSGSGSG |
| | (K-ro22-6) | TEFTLTISSLQPEDFATYYCHQYLSSYTFGQGTKLEIK |
| | kappa_huCD28 | RTVAAPSVFIFPPSDEQLKSGTASVVCLLNNFYPREA |
| | (9.3-8_Vh-Vl) | KVQWKVDNALQSGNSQESVTEQDSKDSTYSLSSTLT |
| | | LSKADYEKHKVYACEVTHQGLSSPVTKSFNRGECAS |
| | | GGGGSGGGGSGGGGSQVQLVESGGGVVQPGRSLRL |
| | | SCAASGFSLSDYGVHWVRQAPGKGLEWLGVIWAGG |
| | | GTNYNSALMSRKTISKDNSKNTVYLQMNSLRAEDT |
| | | AVYYCARDKGYSYYYSMDYWGQGTTVTVSSGGGGS |
| | | GGGGSGGGGSDIQMTQSPSTLSASVGDRVTIICRASE |
| | | SVEYYVTSLMQWYQQKPGKAPKLLIFAASNVESGVP |
| | | SRFSGSGSGTEFTLTISSLQPDDFATYYCQQSRKVPYT |
| | | FGQGTKLEIKR |
| 4 | Heavy chain (Hc- | QVQLVQSGAEVKKPGASVKVSCKASGYTFTSYWMH |
| | L)_hu Endoglin (K- | WVRQAPGQGLEWIGNIYPGSGSTFYDEKFKGRATLT |
| | ro22-2)_huCD28 | VDTSISTAYMELSRLRSDDTAVYYCTRRNYVTGFDY |
| | (9.3-8_Vh- | WGQGTTVTVSSASTKGPSVFPLAPSSKSTSGGTAALG |
| | Vl)_FcKO | CLVKDYFPEPVTVSWNSGALTSGVHTFPAVLQSSGL |
| | | YSLSSVVTVPSSSLGTQTYICNVNHKPSNTKVDKKVE |
| | | PKSCDKTHTCPPCPAPPVAGPSVFLFPPKPKDTLMIS |
| | | RTPEVTCVVVGVSHEDPEVKFNWYVDGVEVHNAKT |
| | | KPREEQYNSTYRVVSVLTVLHQDWLNGKEYKCKVS |
| | | NKQLPSPIEKTISKAKGQPREPQVYTLPPSRDELTKN |
| | | QVSLTCLVKGFYPSDIAVEWESNGQPENNYKTTPPV |
| | | LDSDGSFFLYSKLTVDKSRWQQGNVFSCSVMHEAL |
| | | HNHYTQKSLSLSPGKASQVQLVESGGGVVQPGRSLR |
| | | LSCAASGFSLSDYGVHWVRQAPGKGLEWLGVIWAG |
| | | GGTNYNSALMSRKTISKDNSKNTVYLQMNSLRAED |
| | | TAVYYCARDKGYSYYYSMDYWGQGTTVTVSSGGGG |
| | | SGGGGSGGGGSDIQMTQSPSTLSASVGDRVTIICRAS |
| | | ESVEYYVTSLMQWYQQKPGKAPKLLIFAASNVESGV |
| | | PSRFSGSGSGTEFTLTISSLQPDDFATYYCQQSRKVPY |
| | | TFGQGTKLEIKR |
| 5 | Heavy chain | QVQLVQSGAEVKKPGASVKVSCKASGYTFTSYWMH |
| | (Hc + L)_hu | WVRQAPGQGLEWIGNIYPGSGSTFYDEKFKGRATLT |
| | Endoglin (K-ro22- | VDTSISTAYMELSRLRSDDTAVYYCTRRNYVTGFDY |
| | 2)_huCD28 (9.3- | WGQGTTVTVSSASTKGPSVFPLAPSSKSTSGGTAALG |
| | 8_Vh-Vl)_FcKo | CLVKDYFPEPVTVSWNSGALTSGVHTFPAVLQSSGL |
| | | YSLSSVVTVPSSSLGTQTYICNVNHKPSNTKVDKKVE |
| | | PKSCDKTHTCPPCPAPPVAGPSVFLFPPKPKDTLMIS |
| | | RTPEVTCVVVGVSHEDPEVKFNWYVDGVEVHNAKT |
| | | KPREEQYNSTYRVVSVLTVLHQDWLNGKEYKCKVS |
| | | NKQLPSPIEKTISKAKGQPREPQVYTLPPSRDELTKN |
| | | QVSLTCLVKGFYPSDIAVEWESNGQPENNYKTTPPV |
| | | LDSDGSFFLYSKLTVDKSRWQQGNVFSCSVMHEAL |
| | | HNHYTQKSLSLSPGKASGGGGSGGGGSGGGGSQVQ |
| | | LVESGGGVVQPGRSLRLSCAASGFSLSDYGVHWVRQ |
| | | APGKGLEWLGVIWAGGGTNYNSALMSRKTISKDNS |
| | | KNTVYLQMNSLRAEDTAVYYCARDKGYSYYYSMDY |
| | | WGQGTTVTVSSGGGGSGGGGSGGGGSDIQMTQSPS |
| | | TLSASVGDRVTIICRASESVEYYVTSLMQWYQQKPG |
| | | KAPKLLIFAASNVESGVPSRFSGSGSGTEFTLTISSLQ |
| | | PDDFATYYCQQSRKVPYTFGQGTKLEIKR |
| 6 | Light chain_hu | DIQLTQSPSFLSASVGDRVTITCKSSQSVLYSSNQKNY |
| | Endoglin (K-ro22- | LAWYQQKPGKAPKLLIYWASTRESGVPSRFSGSGSG |
| | 6)_kappa | TEFTLTISSLQPEDFATYYCHQYLSSYTFGQGTKLEIK |
| | | RTVAAPSVFIFPPSDEQLKSGTASVVCLLNNFYPREA |
| | | KVQWKVDNALQSGNSQESVTEQDSKDSTYSLSSTLT |
| | | LSKADYEKHKVYACEVTHQGLSSPVTKSFNRGEC |
| 7 | Heavy chain | QVQLVESGGGVVQPGRSLRLSCAASGFSLSDYGVHW |
| | (Hc + L)_huCD28 | VRQAPGKGLEWLGVIWAGGGTNYNSALMSRKTISK |
| | (9.3-8)_hu Endoglin | DNSKNTVYLQMNSLRAEDTAVYYCARDKGYSYYYS |
| | (K-ro22-2-6_Vh- | MDYWGQGTTVTVSSASTKGPSVFPLAPSSKSTSGGT |
| | Vl)_FcKo | AALGCLVKDYFPEPVTVSWNSGALTSGVHTFPAVLQ |
| | | SSGLYSLSSVVTVPSSSLGTQTYICNVNHKPSNTKVD |
| | | KKVEPKSCDKTHTCPPCPAPPVAGPSVFLFPPKPKDT |
| | | LMISRTPEVTCVVVGVSHEDPEVKFNWYVDGVEVH |
| | | NAKTKPREEQYNSTYRVVSVLTVLHQDWLNGKEYK |
| | | CKVSNKQLPSPIEKTISKAKGQPREPQVYTLPPSRDE |
| | | LTKNQVSLTCLVKGFYPSDIAVEWESNGQPENNYKT |
| | | TPPVLDSDGSFFLYSKLTVDKSRWQQGNVFSCSVMH |
| | | EALHNHYTQKSLSLSPGKASGGGGSGGGGSGGGGS |
| | | QVQLVQSGAEVKKPGASVKVSCKASGYTFTSYWHW |
| | | VRQAPGQGLEWIGNIYPGSGSTFYDEKFKGRATLTV |
| | | DTSISTAYMELSRLRSDDTAVYYCTRRNYVTGFDYW |
| | | GQGTTVTVSSGGGGSGGGGSGGGGSDIQLTQSPSFL |
| | | SASVGDRVTITCKSSQSVLYSSNQKNYLAWYQQKPG |
| | | KAPKLLIYWASTRESGVPSRFSGSGSGTEFTLTISSLQ |
| | | PEDFATYYCHQYLSSYTFGQGTKLEIK |
| 8 | Heavy chain | QVQLVESGGGVVQPGRSLRLSCAASGFSLSDYGVHW |
| | (Hc + L)_ huCD28 | VRQAPGKGLEWLGVIWAGGGTNYNSALMSRKTISK |
| | (9.3-8)_hu Endoglin | DNSKNTVYLQMNSLRAEDTAVYYCARDKGYSYYYS |
| | (K-r022-2-6_Vl- | MDYWGQGTTVTVSSASTKGPSVFPLAPSSKSTSGGT |
| | Vh)_FcKo | AALGCLVKDYFPEPVTVSWNSGALTSGVHTFPAVLQ |
| | | SSGLYSLSSVVTVPSSSLGTQTYICNVNHKPSNTKVD |
| | | KKVEPKSCDKTHTCPPCPAPPVAGPSVFLFPPKPKDT |
| | | LMISRTPEVTCVVVGVSHEDPEVKFNWYVDGVEVH |
| | | NAKTKPREEQYNSTYRVVSVLTVLHQDWLNGKEYK |
| | | CKVSNKQLPSPIEKTISKAKGQPREPQVYTLPPSRDE |
| | | LTKNQVSLTCLVKGFYPSDIAVEWESNGQPENNYKT |
| | | TPPVLDSDGSFFLYSKLTVDKSRWQQGNVFSCSVMH |
| | | EALHNHYTQKSLSLSPGKASGGGGGGGGSGGGGS |
| | | DIQLTQSPSFLSASVGDRVTITCKSSQSVLYSSNQKNY |
| | | LAWYQQKPGKAPKLLIYWASTRESGVPSRFSGSGSG |
| | | TEFTLTISSLQPEDFATYYCHQYLSSYTFGQGTKLEIK |
| | | GGGGSGGGGSGGGGSQVQLVQSGAEVKKPGASVKV |
| | | SCKASGYTFTSYWMHWVRQAPGQGLEWIGNIYPGS |
| | | GSTFYDEKFKGRATLTVDTSISTAYMELSRLRSDDTA |
| | | VYYCTRRNYVTGFDYWGQGTTVTVSS |
| 9 | Light chain_ | DIQMTQSPSTLSASVGDRVTIICRASESVEYYVTSLM |
| | huCD28 (9.3- | QWYQQKPGKAPKLLIFAASNVESGVPSRFSGSGSGT |
| | 8)_kappa | EFTLTISSLQPDDFATYYCQQSRKVPYTFGQGTKLEI |
| | | KRRTVAAPSVFIFPPSDEQLKSGTASVVCLLNNFYPR |
| | | EAKVQWKVDNALQSGNSQESVTEQDSKDSTYSLSST |
| | | LTLSKADYEKHKVYACEVTHQGLSSPVTKSFNRGEC |
| 10 | Heavy chain_B7- | EVQLEQSGPELVKPGTSVKISCKTSGYTFTEYTMHW |
| | H3(7C4)_huBMA03 | VKQSHGKSLEWIGGINPNNGGTTYNQIFKNKATLTV |
| | 1.2.1_VhVl_FcKo | DKSSSTAYMELRSLTSEDSAVYYCARRGYHVSSWYF |
| | | DVWGAGTTVTVSSASTKGPSVFPLAPSSKSTSGGTAA |
| | | LGCLVKDYFPEPVTVSWNSGALTSGVHTFPAVLQSS |
| | | GLYSLSSVVTVPSSSLGTQTYICNVNHKPSNTKVDKK |
| | | VEPKSCDKTHTCPPCPAPPVAGPSVFLFPPKPKDTL |
| | | MISRTPEVTCVVVGVSHEDPEVKFNWYVDGVEVHN |
| | | AKTKPREEQYNSTYRVVSVLTVLHQDWLNGKEYKC |
| | | KVSNKQLPSPIEKTISKAKGQPREPQVYTLPPSRDEL |
| | | TKNQVSLTCLVKGFYPSDIAVEWESNGQPENNYKTT |
| | | PPVLDSDGSFFLYSKLTVDKSRWQQGNVFSCSVMH |
| | | EALHNHYTQKSLSLSPGKSGQVQLVQSGAEVKKPGA |
| | | SVKVSCKASGYTFTSYVMHWVRQAPGQGLEWIGYI |
| | | NPYNDVTKYNEKFKGRVTMTRDTSTSTVYMELSSLR |
| | | SEDTAVYYCARGSYYDYDGFVYWGQGTLVTVSSGGG |
| | | GSGGGGSGGGGSEIVLTQSPATLSLSPGERATLSCSA |
| | | TSSVSYMHWYQQKPGLAPRRWIYDTSKLASGIPDRF |
| | | SGSGSGTDFTLTISRLEPEDFAVYYCQQWSSNPLTFG |
| | | AGTKLELK |
| 11 | Light chain_B7-H3 | DIVLTQSTAIMSASPGEKVTMTCSASSSVSYMHWYQ |
| | (7C4)_kappa | QKSGTSPKRWIYDTSKLASGVPARFSGSGSGTSYSLT |
| | | ISSMETEDSATYYCLQWHSNPLTFGAGTKLELKRTV |
| | | AAPSVFIFPPSDEQLKSGTASVVCLLNNFYPREAKVQ |
| | | WKVDNALQSGNSQESVTEQDSKDSTYSLSSTLTLSK |
| | | ADYEKHKVYACEVTHQGLSSPVTKSFNRGEC |
| 12 | Heavy chain_huEng | QVQLVQSGAEVKKPGASVKVSCKASGYTFTSYWMH |
| | (Kro-22.2) Fc | WVRQAPGQGLEWIGNIYPGSGSTFYDEKFKGRATLT |
| | silenced optimized | VDTSISTAYMELSRLRSDDTAVYYCTRRNYVTGFDY |
| | (see FIG. 12) | WGQGTTVTVSSASTKGPSVFPLAPSSKSTSGGTAALG |
| | | CLVKDYFPEPVTVSWNSGALTSGVHTFPAVLQSSGL |
| | | YSLSSVVTVPSSSLGTQTYICNVNHKPSNTKVDKKVE |
| | | PKSCDKTHTCPPCPAPEAAGGPSVFLFPPKPKDTLYI |
| | | TREPEVTCVVVDVSHEDPEVKFNWYVDGVEVHNAK |
| | | TKPREEQYNSTYRVVSVLTVLHQDWLNGKEYKCKV |
| | | SNKALPAPIEKTISKAKGQPREPQVYTLPPSRDELTK |
| | | NQVSLTCLVKGFYPSDIAVEWESNGQPENNYKTTPP |
| | | VLDSDGSFFLYSKLTVDKSRWQQGNVFSCSVMHEA |
| | | LHNHYTQKSLSLSPGK |
| 13 | LC + L (Light | DIQLTQSPSFLSASVGDRVTITCKSSQSVLYSSNQKNY |
| | chain)_huEng (Kro- | LAWYQQKPGKAPKLLIYWASTRESGVPSRFSGSGSG |
| | 22.6)_kappa_ | TEFTLTISSLQPEDFATYYCHQYLSSYTFGQGTKLEIK |
| | CD28(hu9.3.8.V1) | RTVAAPSVFIFPPSDEQLKSGTASVVCLLNNFYPREA |
| | optimized (see | KVQWKVDNALQSGNSQESVTEQDSKDSTYSLSSTLT |
| | FIG. 12) | LSKADYEKHKVYACEVTHQGLSSPVTKSFNRGECAS |
| | | GGGGSGGGGSGGGGSGGSQVQLVQSGGGVVQPGQS |
| | | LRLSCAASGFSLSDYGVHWVRQAPGKGLEWLGVIW |
| | | AGGGTNYNSALQSRFTISKDNSKNTVYLQMNSLRAE |
| | | DTAVYYCARDKGYSYYYSMDYWGQGTTVTVSSGGG |
| | | GSGGGGSGGGGSGGSDIQMTQSPSTLSASVGDRVTI |
| | | TCRASESVEYYVTSLMQWYQQKPGKAPKLLIFAASN |
| | | VESGVPSRFSGSGSGTEFTLTISSLQPDDFATYYCQQS |
| | | RKVPYTFGQGTKLEIK |
| 24 | Optimized anti- | QVQLVQSGGGVVQPGQSLRLSCAASGFSLSDYGVH |
| | CD28 scFv | WVRQAPGKGLEWLGVIWAGGGTNYNSALQSRFTIS |
| | | KDNSKNTVYLQMNSLRAEDTAVYYCARDKGYSYYYS |
| | | MDYWGQGTTVTVSSGGGGSGGGGSGGGGSGGSDIQ |
| | | MTQSPSTLSASVGDRVTITCRASESVEYYVTSLMQW |
| | | YQQKPGKAPKLLIFAASNVESGVPSRFSGSGSGTEFT |
| | | LTISSLQPDDFATYYCQQSRKVPYTFGQGTKLEIK |

TABLE 2
Endoglin Antibody sequences:

| SEQ | | | | |
|---|---|---|---|---|
| ID | Antibody | | Sequence | |
| NO: | Clone | Region | type | Sequence |
| 14 | Endoglin | HCDR1 | amino | SYWMH |
| | humanized | | acid | |
| | AB Clone | | | |
| | Kro22.2 | | | |
| 15 | Endoglin | HCDR2 | amino | NIYPGSGSTFYDEKFKG |
| | humanized | | acid | |
| | AB Clone | | | |
| | Kro22.2 | | | |
| 16 | Endoglin | HCDR3 | amino | RNYVTGFDY |
| | humanized | | acid | |
| | AB Clone | | | |
| | Kro22.2 | | | |
| 17 | Endoglin | Heavy | amino | QVQLVQSGAEVKKPGASVKVSCKASG |
| | humanized | chain | acid | YTFTSYWMHWVRQAPGQGLEWIGNI |
| | AB Clone | variable | | YPGSGSTFYDEKFKGRATLTVDTSIST |
| | Kro22.2 | (full) | | AYMELSRLRSDDTAVYYCTRRNYVTG |
| | | | | FDYWGQGTTVTVSS |
| 18 | Endoglin | LCDR1 | amino | KSSQSVLYSSNQKNYLA |
| | humanized | | acid | |
| | AB Clone | | | |
| | Kro22.6 | | | |
| 19 | Endoglin | LCDR2 | amino | WASTRES |
| | humanized | | acid | |
| | AB Clone | | | |
| | Kro22.6 | | | |
| 20 | Endoglin | LCDR3 | amino | HQYLSSYT |
| | humanized | | acid | |
| | AB Clone | | | |
| | Kro22.6 | | | |
| 21 | Endoglin | Light | amino | DIQLTQSPSFLSASVGDRVTITCKSSQS |
| | humanized | chain | acid | VLYSSNQKNYLAWYQQKPGKAPKLLI |
| | AB Clone | variable | | YWASTRESGVPSRFSGSGSGTEFTLTI |
| | Kro22.6 | (full) | | SSLQPEDFATYYCHQYLSSYTFGQGTK |
| | | | | LEIK |
| 22 | Endoglin | Heavy | nucleic | CAGGTTCAGCTGGTTCAGTCTGGCGC |
| | humanized | chain | acid | CGAAGTGAAGAAACCTGGCGCCTCTG |
| | AB Clone | variable | | TGAAGGTGTCCTGCAAGGCCAGCGGC |
| | Kro22.2 | (full) | | TACACCTTTACCAGCTACTGGATGCA |
| | | | | CTGGGTCCGACAGGCTCCAGGACAAG |
| | | | | GCCTGGAATGGATCGGCAACATCTAC |
| | | | | CCTGGCAGCGGCAGCACCTTCTACGA |
| | | | | CGAGAAGTTCAAGGGCAGAGCCACAC |
| | | | | TGACCGTGGACACCAGCATCAGCACC |
| | | | | GCCTACATGGAACTGAGCCGGCTGAG |
| | | | | ATCCGATGACACCGCCGTGTACTACT |
| | | | | GCACCAGACGGAATTACGTGACCGGC |
| | | | | TTCGACTATTGGGGCCAGGGCACAAC |
| | | | | CGTGACCGTTAGCTCT |
| 23 | Endoglin | Light | nucleic | GACATCCAGCTGACTCAGAGCCCTAG |
| | humanized | chain | acid | CTTTCTGAGCGCCAGCGTGGGCGACA |
| | AB Clone | variable | | GAGTGACCATCACCTGTAAAAGCAGC |
| | Kro22.6 | (full) | | CAGAGCGTGCTGTACTCCAGCAACCA |
| | | | | GAAGAACTACCTGGCCTGGTATCAGC |
| | | | | AGAAGCCCGGCAAGGCTCCTAAGCTG |
| | | | | CTGATCTACTGGGCCAGCACCAGAGA |
| | | | | AAGCGGCGTGCCAAGCAGATTTTCTG |
| | | | | GCAGCGGCTCTGGCACCGAGTTCACC |
| | | | | CTGACCATATCTAGCCTGCAGCCTGA |
| | | | | GGACTTCGCCACCTACTACTGCCACC |
| | | | | AGTACCTGAGCAGCTACACCTTTGGC |
| | | | | CAGGGCACCAAGCTGGAAATCAAG |

### Examples

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

The examples show:

Example 1: A bispecific costimulatory (“BiCo”) antibody having a binding specificity to CD28 and to Endoglin.

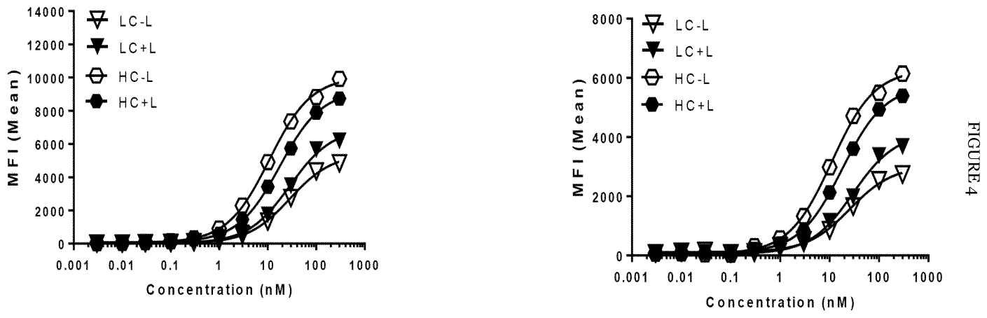

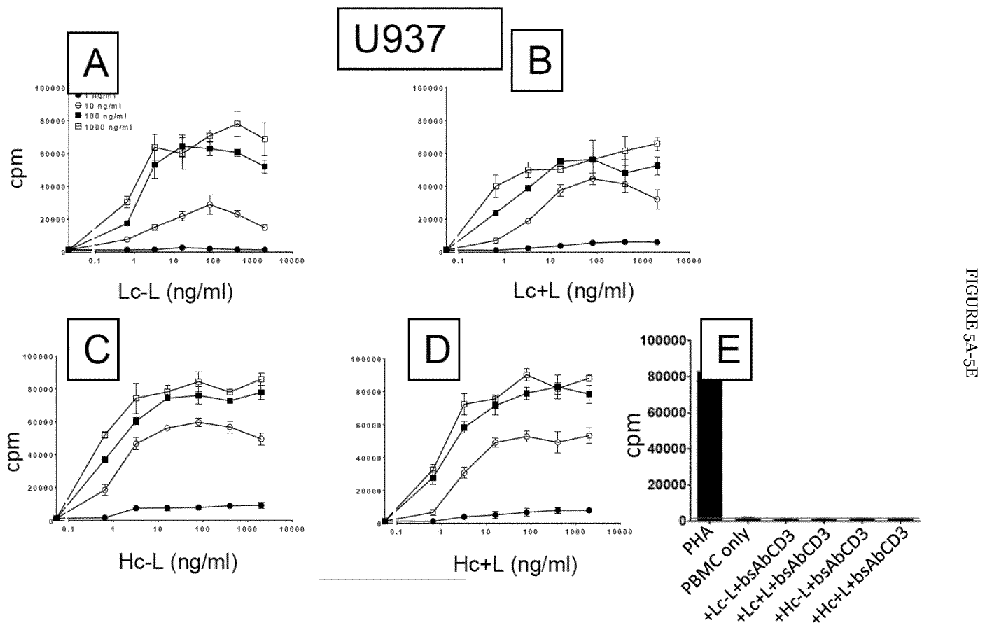

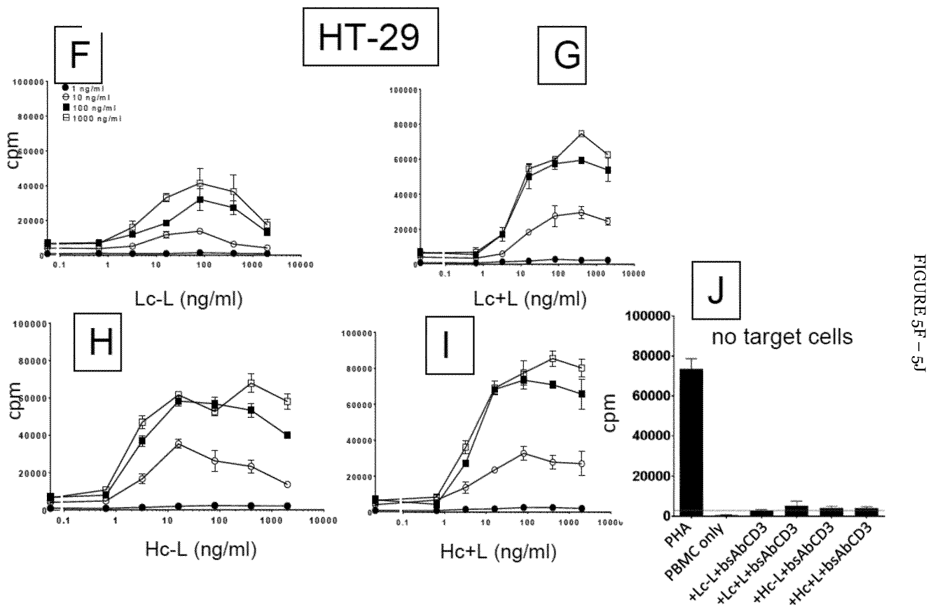

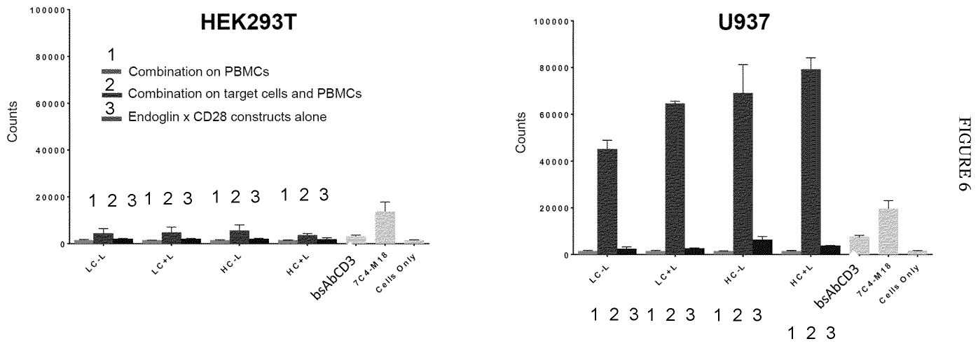

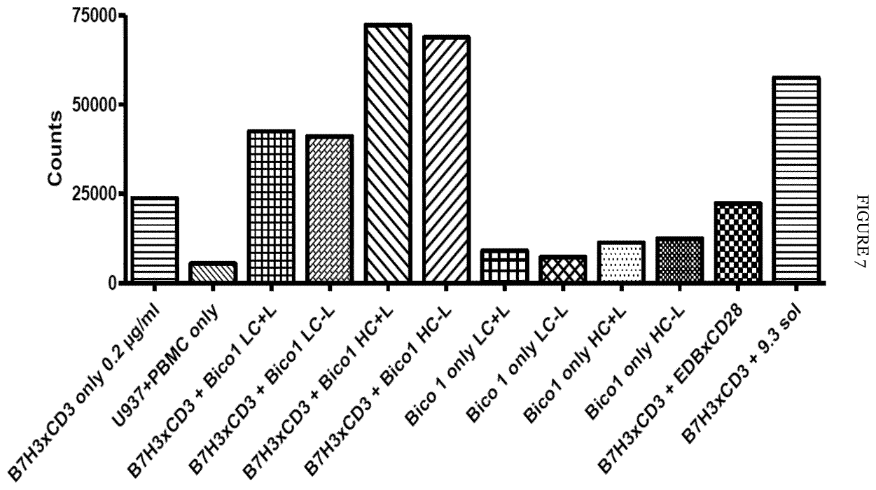

The IgG based molecules used for this invention are—in part—based on the constructs described by Coloma & Morrison (IgGsc-format), where a single chain moiety has been fused after the C-terminus of a normal IgG antibody heavy chain [22]. In addition, the inventors used constructs where the two single chains have been fused to the C-terminus of the light chain. The inventors further added short and long linkers at the fusion sites to allow for variable affinities of the CD28 binding moieties (see FIG. 3).

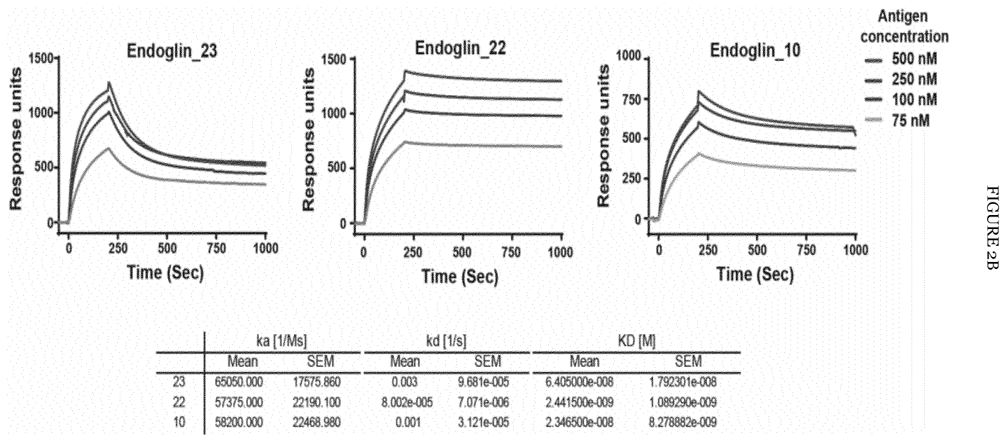

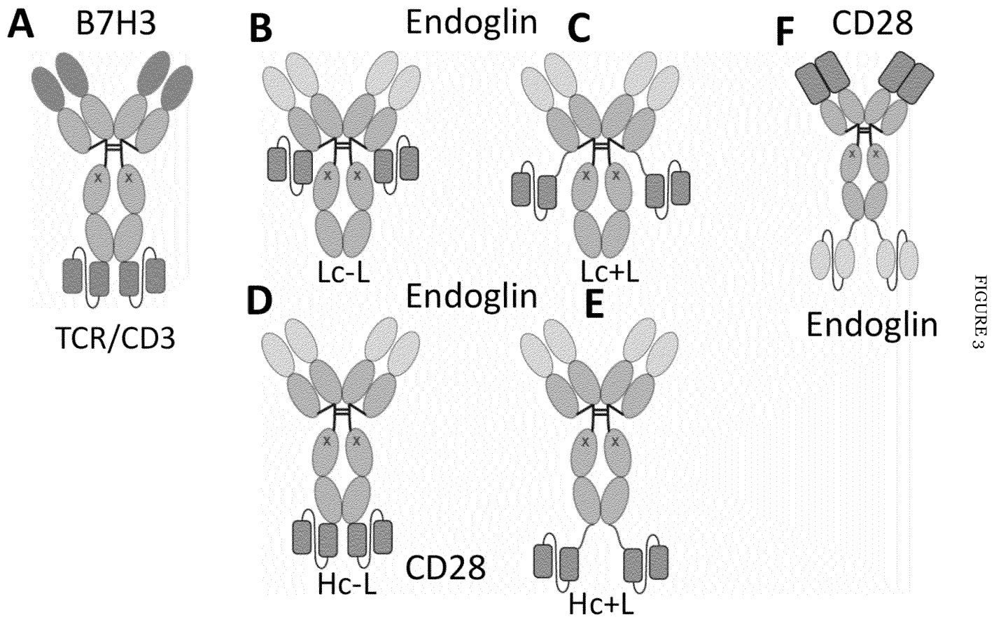

As an example, the TAA antibody in the costimulatory constructs depicted in FIG. 3 is directed against endoglin (CD105), that is expressed on cancer associated fibroblasts as well as on some leukemic cells and, most importantly, on the vasculature of many solid tumors [23]. This reagent has been selected from a panel of newly raised antibodies based on its superior affinity (FIG. 2). The used endoglin clone was sequenced, and the sequences are shown in Table 2. The clone is characterized by an improved binding affinity.

The BiCos were evaluated in combination with a B7H3xTCR/CD3 bsAb (bsAbCD3) directed against a particular epitope of the TCR/CD3 complex providing an attenuated “first signal” for T cell activation. This resulted in a largely CD28 dependent activation and, consequently, in an increased specificity of the combination as outlined above.

The antibodies were tested in co-cultures of human monocyte depleted PBMC (peripheral blood mononuclear cell) cultures and irradiated tumor target cells that exhibited (i) a high or (ii) a low or (iii) undetectable expression of endoglin. Monocyte depleted PBMC were used to prevent binding of the BiCo variants to endoglin expressed on activated cells of the monocytic lineage.

In a first round of experiments it is shown that all BiCo variants greatly enhanced bsAbCD3-induced activation of T cells within PBMC cultures in a target cell restricted manner. Target cell restriction at rather high antibody concentrations was demonstrated by a lack of activity either (i) in the absence of target cells or even more specifically (ii) in the presence of target cells that lack endoglin, the target antigen for the BiCos but express B7H3, the target antigen for the bsAbCD3. Importantly, it was clearly demonstrated in these experiments that only the combination of the two bsAb effectively induces T cell activation, which—due to the demonstrated target cell restriction—is thus dependent on the presence of two target antigens. Neither the bsAbCD3 nor any of the BiCos exerted activity on their own.

In a second round of experiments it is likewise shown that in the presence of target cells BiCo molecules directed to a non-expressed target antigen (extradomain B of fibronectin, EDB) do not exhibit costimulatory activity. To the contrary, the monospecific, bivalent CD28 antibody (present within the BiCo constructs as a C-terminal single chain moiety) exerts striking costimulatory activity irrespective of the expression of any particular antigen on the target cells.

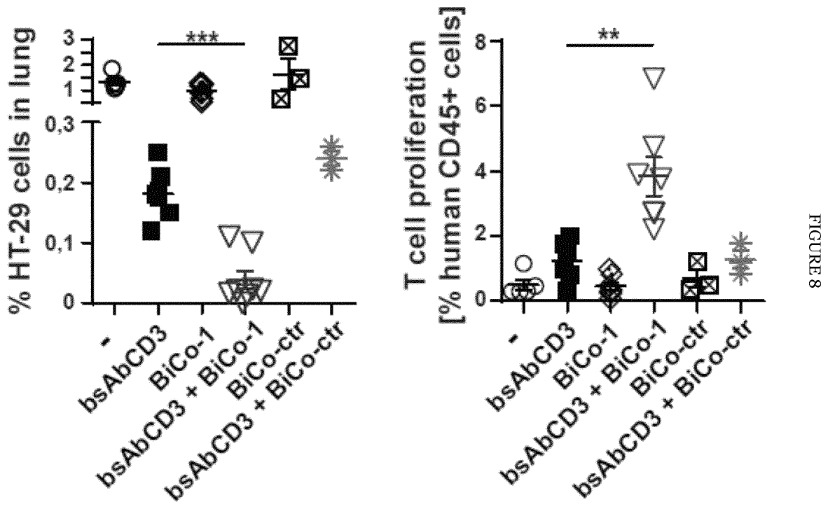

Thus, target cell restriction demonstrated as outlined above are unexpected. This is most strikingly illustrated by the unrestricted costimulatory activity of a particular endoglin targeting BiCo variant Hc+L, that contains the CD28 binding part as an N-terminal Fab2- rather than a C-terminal single chain moiety (see FIGS. 3F and 7).

Finally, it was demonstrated that the BiCo variant Lc+L largely enhanced the anti-tumor activity of bsAbCD3 in immunocompromised mice adoptively transferred with human PBMC (FIG. 8).

Example 2: A bispecific costimulatory (“BiCo-2”) antibody having a binding specificity to CD28 and to B7H3.

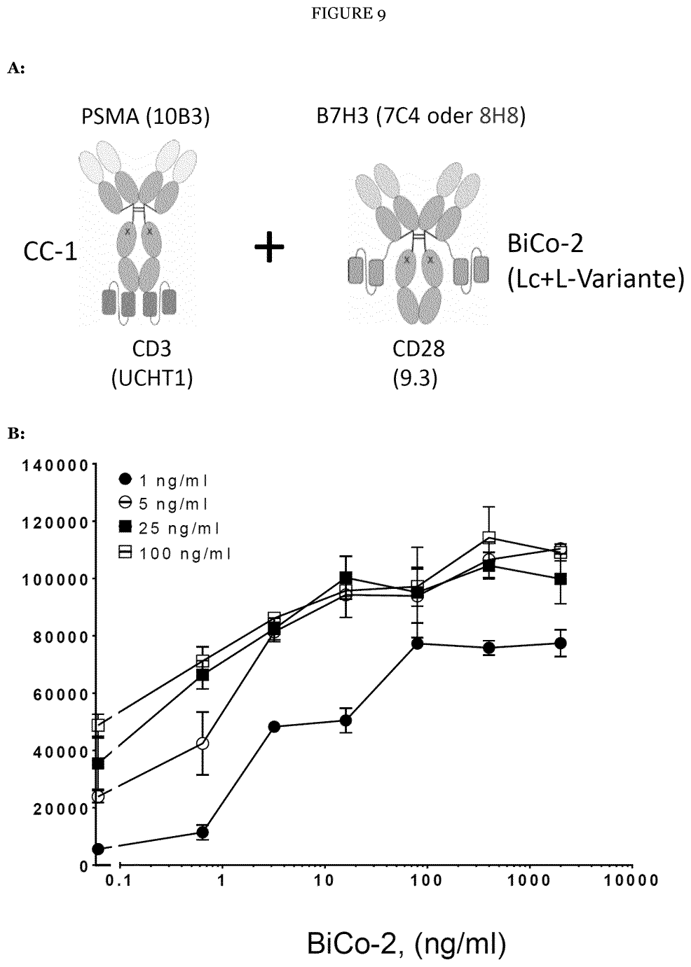

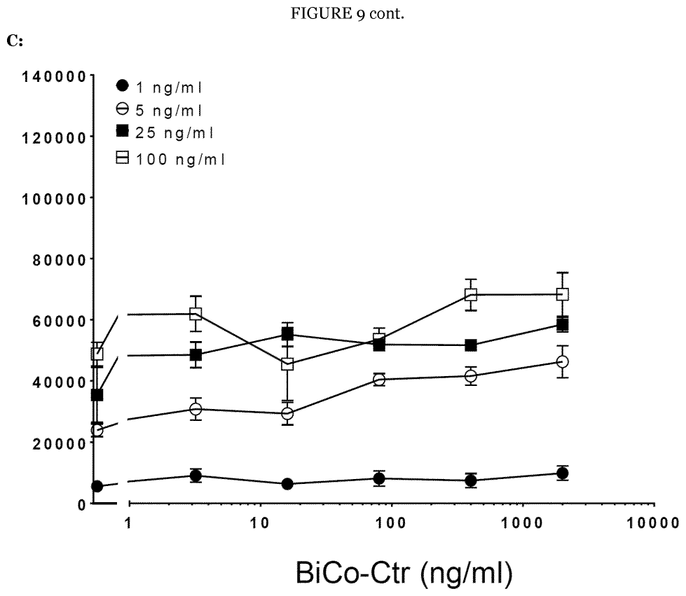

The architecture of the BiCo-2 antibody is shown in FIG. 9A. The binding functionality to B7H3 of the BiCo-2 construct is derived from the antibody clone 7C4 as disclosed in WO 2021/099347 (incorporated herein by reference in its entirety). The bispecific CC-1 construct is described in WO/2017/121905 (incorporated herein by reference in its entirety).

Monocyte depleted PBMC were incubated in a 96 well tissue culture plate (2x10exp5 per well) with irradiated LNCaP prostate carcinoma cells (0,5x10exp5 per well) that express the target antigens PSMA and B7H3 and the indicated antibody concentrations. After 2 days 3 H thymidine (0.5 μCi per well) was added and after additional 20 hrs the plate was harvested, and the radioactivity incorporated into cells was determined using a Micro Beta scintillation counter.

Interestingly, the BiCo-2 construct enhances T cell proliferation induced by CC-1 considerably, while an otherwise identical control antibody directed to an unrelated target antigen does not. The BiCo-2 effect is most pronounced at low CC-1 concentrations (1 ng/ml) where proliferation depends almost completely on BiCo-1. At higher CC-1 concentrations significant T-cell proliferation is induced by CC-1 alone.

Example 3: A further bispecific costimulatory (“BiCo-3”) antibody having a binding specificity to CD28 and to fibroblast activation protein (FAB.

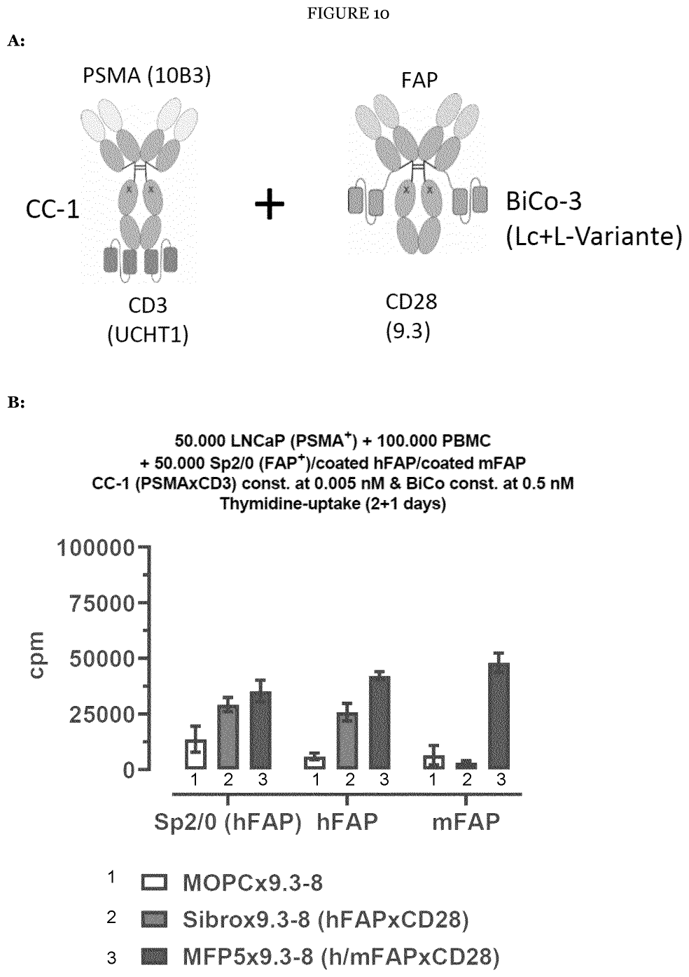

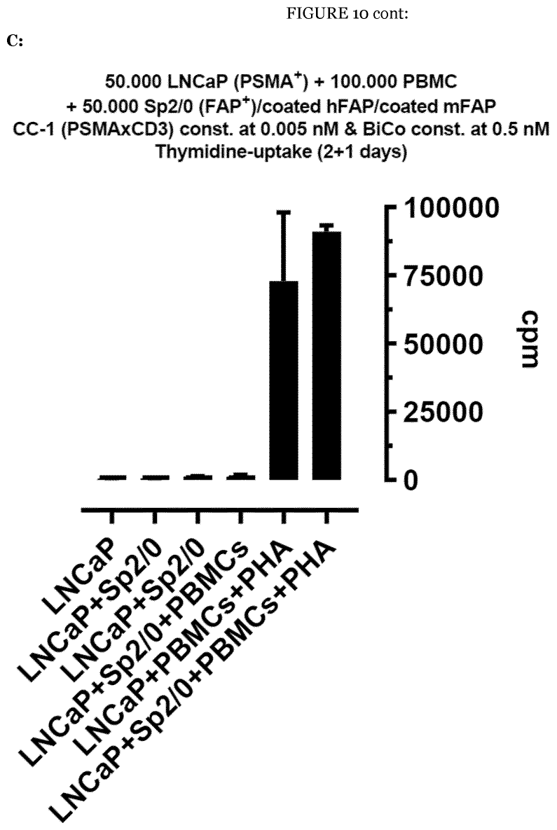

The architecture of the BiCo-3 antibody is shown in FIG. 10A. The binding functionality to FAB of the BiCo-3 construct is derived from the antibody clone Sibrox9.3-8 for hFAB and MFP5x9.3—for the mFAB. The bispecific CC-1 construct is described in WO/2017/121905 (incorporated herein by reference in its entirety).

Human monocyte depleted PBMC (10exp5 per well) were incubated with FAPxCD28 variants at 0,1 82 g/ml and a PSMAxCD3 antibody at 1 ng/ml together with irradiated Sp2/0 cell transfected with human FAP (0,5x10exp5 per well) and irradiated human LNCaP cells expressing PSMA (0,5x10exp5 per well). Alternatively, only irradiated LNCaP cells were used in wells previously coated with 1 μg/ml recombinant human or mouse FAP. After 2 days 3 H thymidine (0.5 μCi per well) was added and after additional 20 hrs the plate was harvested and the radioactivity incorporated into cells was determined using a Micro Beta scintillation counter. Results are depicted in FIG. 10B and C.

The results indicate that the addition of BiCo molecules with FAPxCD28 specificity markedly and specifically enhances T cell proliferation induced by a low concentration of CC-1 even if the different target antigens are presented on separate cells or as coated proteins.

Example 4: Target cell restriction is only possible with a construct having an scFv anti-CD28 in combination with a F(ab) target cell specific antibody in one bispecific costimulatory antibody construct.

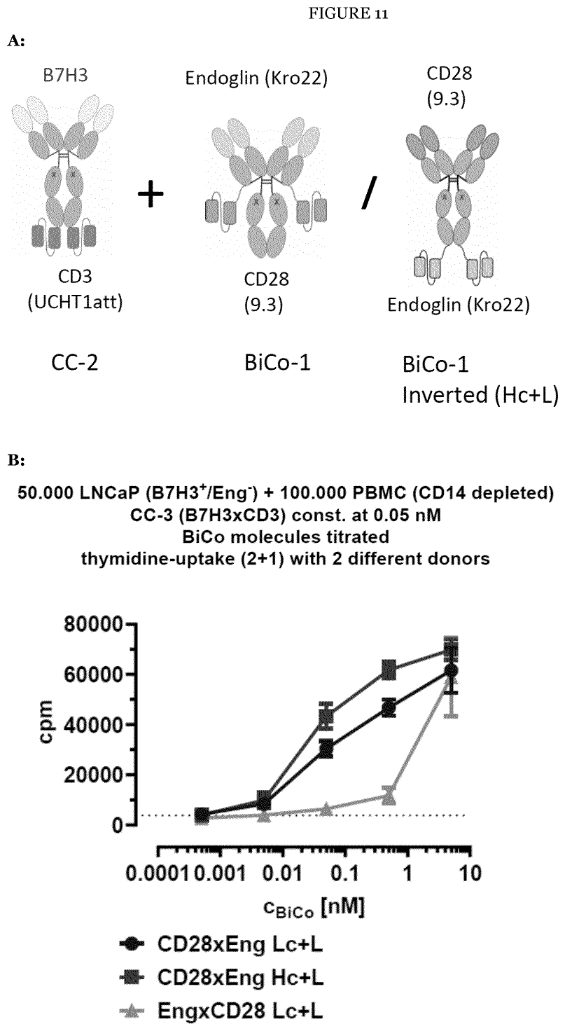

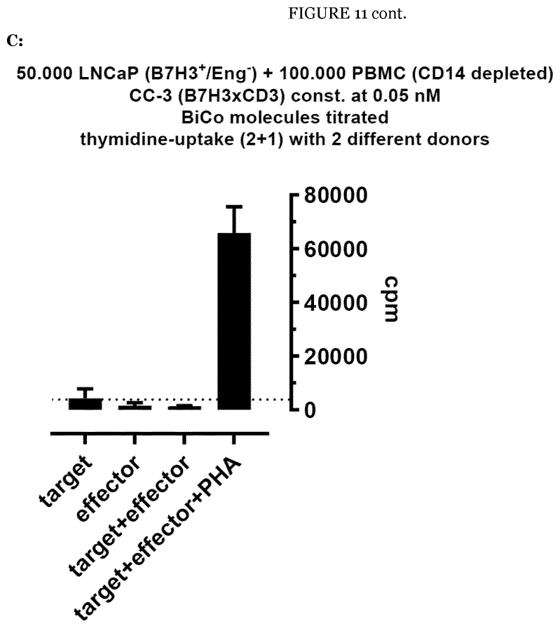

Monocyte depleted PBMC (10exp5 per well) were incubated in a 96 well tissue culture plate with irradiated LNCaP cells (0,5x10exp5 per well), CC-3 (10 ng/ml) and bispecific co-stimulators at the indicated concentrations. After 2 days 3 H thymidine (0.5μCi per well) was added and after additional 20 hrs the plate was harvested, and the radioactivity incorporated into cells was determined using a Micro Beta scintillation counter. Results are depicted in FIG. 11.

Inverted molecules with a natural, n-terminal CD28 binding-part exert marked co-stimulation together with CC-3 although no endoglin to which they might bind is present. For BiCo-1 this type of unspecific and undesired co-stimulation is far less pronounced and detected only at very high antibody concentrations. This result shows that the BiCo architecture as disclosed herein is advantageous over other known bispecific formats.

Example 5: Optimization of BiCo-1 Sequence

The sequence modification for the CD28 scFv (heavy light chain sequencers) as listed in FIG. 12 were introduced, CHO cells were transfected with the respective constructs and proteins were purified by protein A affinity—and size exclusion chromatography using a Superdex S 200 column (A and B). In C % aggregates and production rates of the original—and the modified proteins are compared.

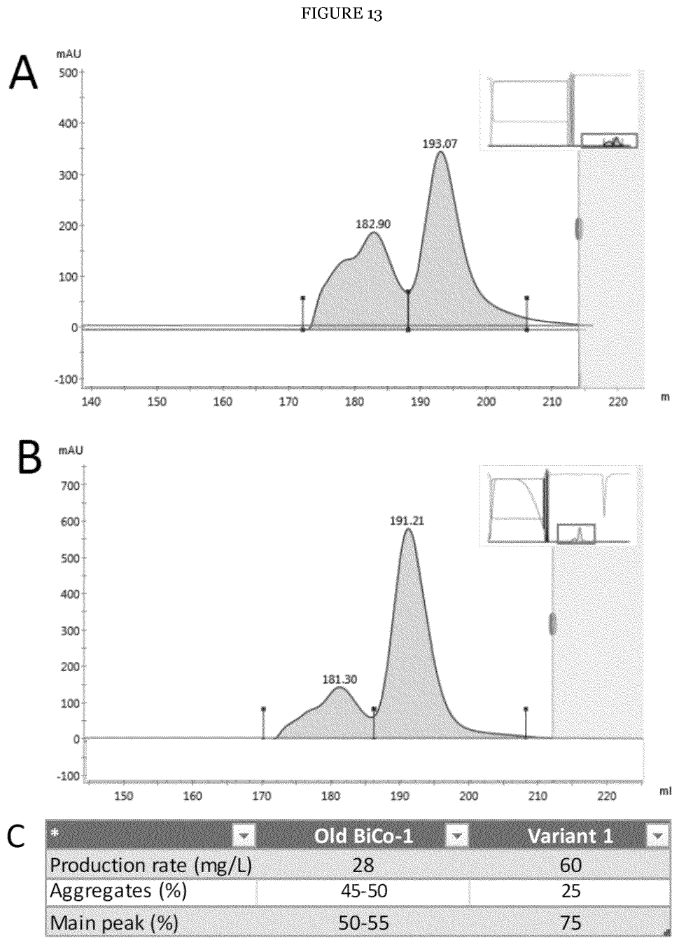

The results of FIG. 13 show that the optimized molecule exhibits a lower aggregation tendency and an improved producibility.

To compare the new variant of the BiCo with the original molecule monocyte depleted PBMC were incubated with irradiated U937 cells expressing B7H3 and endoglin, a bispecific B7H3xCD3 antibody and the old as well as the optimized variant (v1=HV) of the BiCo-1 antibody with endoglinxCD28-specificity. After 2 days 3 H thymidine (0.5 μCi per well) was added and after additional 20 hrs the plate was harvested, and the radioactivity incorporated into cells was determined using a Micro Beta scintillation counter.

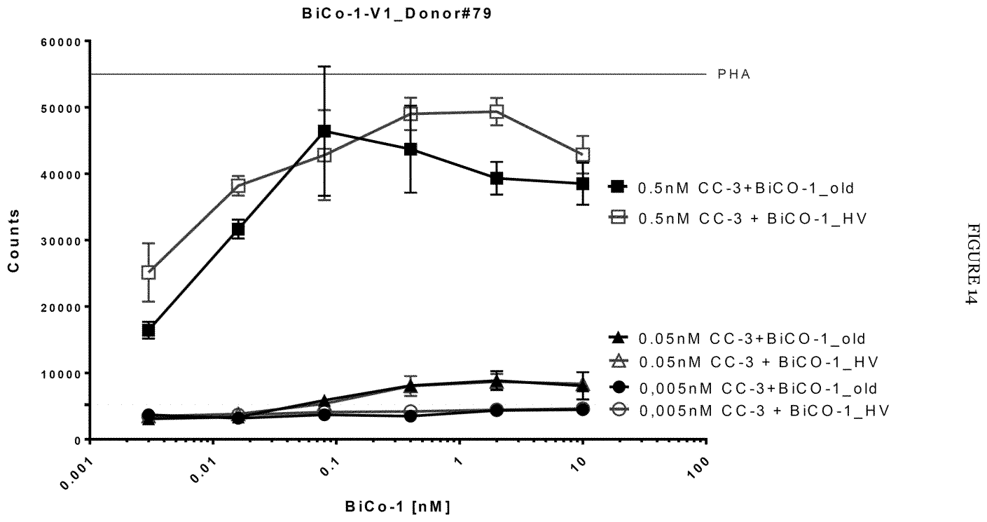

The results shown in FIG. 14, indicate that the T cell proliferation induced by the two BiCo-1 variants was indistinguishable. The sequence variant therefore has comparable biological activity while being less prone to aggregation.

References

The references are:
1. Murphy K, Weaver C (eds.) Janeway's immunobiology, 10th edition. 2016
2. Bretscher P. The two-signal model of lymphocyte activation twenty-one years later. Immunol Today 1992. 13:74
3. Chambers CA, Allison JP. Costimulation in T cell responses. Curr Opin Immunol 1997. 9: 396:
4. Wolf H, Müller Y, Salmen S, Wilmanns W and Jung G. Induction of anergy in resting human T-lymphocytes by immobilized anti-CD3 antibodies. Eur J Immuno 11994. 24:1410
5. Radvanyi LG, Shi Y, Vaziri H, Sharma A, Dhala R, Mills GB, Miller RG. CD28 costimulation inhibits TCR-induced apoptosis during a primary T cell response. J Immunol 1996. 156:1788
6. Slaney CY, Wang P, Darcy PK, Kershaw MH. CARs versus BiTEs: A Comparison between T Cell-Redirection Strategies for Cancer Treatment. Cancer Discov 2018. 8:924.
7. Gross G, Eshhar Z (2016) Therapeutic Potential of T Cell Chimeric Antigen Receptors (CARs) in Cancer Treatment: Counteracting Off-Tumor Toxicities for Safe CAR T Cell Therapy. Annu Rev Pharmacol Toxicol 2016. 56: 59
8. Riethmuller G. Symmetry breaking: bispecific antibodies, the beginnings, and 50 years on. Cancer Immun 2012. 12:12
9. Schuster SJ, Bishop MR, Tam CS, et al. Tisagenlecleucel in Adult Relapsed or Refractory Diffuse Large B-Cell Lymphoma. N Engl J Med 2019; 380:45
10. Beatty GL, O'Hara M. Chimeric antigen receptor-modified T cells for the treatment of solid tumors: Defining the challenges and next steps. Pharmacol Ther 2016. 166: 30
11. Gross G, Waks T, Eshhar Z. Expression of immunoglobulin-T-cell receptor chimeric molecules as functional receptors with antibody-type specificity. Proc Natl Acad Sci USA 1989. 86:10024
12. Perez P, Hoffman RW, Shaw S, Bluestone JA, Segal DM. Specific targeting of cytotoxic T cells by anti-T3 linked to anti-target cell antibody. Nature 1985. 316:354
13. Staerz UD, Kanagawa O, Bevan MJ. Hybrid antibodies can target sites for attack by T cells. Nature 1985. 314: 628
14. Maude SL, Barrett D, Teachey DT, Grupp SA. Managing cytokine release syndrome associated with novel T cell-engaging therapies. Cancer J 2014. 20:119
15. Pang Y, Hou X, Yang C, Liu Y, Jiang G. Advances on chimeric antigen receptor-modified T-cell therapy for oncotherapy. Mol Cancer 2018. 17: 91
16. Jung G, Ledbetter JA, Muller-Eberhard HJ. Induction of cytotoxicity in resting human T lymphocytes bound to tumor cells by antibody heteroconjugates. Proc Natl Acad Sci USA 1987. 84:4611
17. Hunig T (2012) The storm has cleared: lessons from the CD28 superagonist TGN1412 trial. Nat Rev Immunol 12: 317-318
18. Ayyar BV, Arora S, O'Kennedy R. Coming-of-Age of Antibodies in Cancer Therapeutics. Trends Pharmacol Sci 2016. 37:1009
19. Manger B, Weiss A, Weyand C, Goronzy J, Stobo JD. T cell activation: differences in the signals required for IL 2 production by nonactivated and activated T cells. J Immunol 1985. 135:3669
20. Weiss A, Manger B, Imboden J. Synergy between the T3/antigen receptor complex and Tp44 in the activation of human T cells. J Immunol. 1986. 137:819
21. Parker KR, Migliorini D, Perkey E, Yost KE, Bhaduri A, Bagga P, Haris M, Wilson NE, Liu F, Gabunia K, Scholler J, Montine TJ, Bhoj VG, Reddy R, Mohan S, Maillard I, Kriegstein AR, June CH, Chang HY, Posey AD Jr, Satpathy AT. Single-Cell Analyses Identify Brain Mural Cells Expressing CD19 as Potential Off-Tumor Targets for CAR-T Immunotherapies. Cell. 2020. 183:126
22. Coloma MJ, Morrison SL. Design and production of novel tetravalent bispecific antibodies. Nat Biotechnol 1997. 15:159
23. Schoonderwoerd MJA, Goumans MTH, Hawinkels LJAC. Endoglin: Beyond the Endothelium. Biomolecules 2020. 10:289

1-30. (canceled).

31. A method for treatment of a disease in a subject, comprising administering a binding molecule which is at least bispecific comprising at least two first antigen binding sites and at least one second antigen binding site, wherein
(i) the at least two first antigen binding sites are capable of specifically binding to an epitope of T-cell-specific surface glycoprotein CD28 and wherein each are provided as an antigen binding fragment of an antibody and which is not a Fab, F(ab′)2 or IgG; and
(ii) the at least one second antigen binding site is capable of binding to an epitope of an antigenic target protein expressed on or in a cell associated with the disease in the subject and wherein the at least one second antigen binding site(s) is derived from an antibody or antibody like molecule and comprises a Fab, F(ab′)2 or IgG.

32. The method of claim 31, wherein the at least two first antigen binding sites are each provided as a single chain Fv (scFv).

33. The method of claim 31, wherein the binding molecule when contacted with a first cell that is a CD28 positive immune cell (such as a T-cell) in absence of a second cell expressing the antigenic target protein, does not induce CD28 signaling.

34. The method of claim 31, wherein the antigenic target protein is selected from a protein expressed on cells associated with a proliferative disorder, a protein or other molecule associated with a pathogenic organism.

35. The method of claim 31, wherein the antigenic target protein is endoglin, FAB or B7H2.

36. The method of claim 31, wherein each one of the at least two first antigen binding sites is directly connected with the at least one second antigen binding site.

37. The method of claim 36, wherein the binding molecule comprises an equal number of first and second antigen binding sites, and wherein one of the at least two first antigen binding sites is terminally connected (via a peptide bond) to the light chain (or alternatively the heavy chain) of one of the at least two second antigen binding sites, and wherein the other of the at least two first antigen binding sites is terminally connected (via a peptide bond) to the light chain (or alternatively the heavy chain) of the other of the at least two second antigen binding sites.

38. The method of claim 31, comprising at least two second antigen binding sites.

39. The method of claim 31, wherein the binding molecule comprises exactly, and not more than, two first antigen binding sites, and exactly, and not more than, one or two second antigen binding sites.

40. The method of any one of claim 31, wherein the at least two first antigen binding sites bind the same epitope on CD28.

41. The method of claim 31, wherein at least one of the at least two first antigen binding sites and the at least one second antigen binding site are linked to each other by a protein-linker comprising one or more antibody-derived human constant domains.

42. The method of claim 31, wherein the at least two first antigen binding sites comprise an antibody heavy chain sequence and an antibody light chain sequence, each derived from, and competitively binding to the same antigen as, the C-terminal binding site comprised in an antibody sequence shown in SEQ ID NO: 2, 3, 4, 5, 7, 8 or 9.

43. The method of claim 31, wherein the at least one second antigen binding site comprises an antibody heavy chain sequence and an antibody light chain sequence, each derived from, and competitively binding to the same antigen as, the N-terminal binding site comprised in an antibody composed of SEQ ID NO: 1 and 2.

44. The method of claim 31, wherein the binding molecule specifically binds to an immune cell and a cell associated with the disease, wherein the immune cell is an immune cell involved with a cell-mediated immune response.

45. The method of claim 31, wherein the immune cell is a cell expressing CD28 and CD3 and a TCR.

46. The method of claim 31, wherein the subject is characterized in that CD3/TCR signalling is activated by treatment, or endogenously in response to a disease-associated antigen.

47. The method of claim 31, wherein the treatment further comprises stimulation and/or activation of immune cells towards cells associated with the disease.

48. The method of claim 31, comprising two antibody heavy chain sequences, and two antibody light chain sequences, and wherein
(iii) One of the at least two first antigen binding sites is covalently connected to a C-terminal end of one of the two antibody light chain sequences, and the other of the at least two first antigen binding sites is covalently connected to a C-terminal end of the other of the two antibody light chain sequences; or
(iv) One of the at least two first antigen binding sites is covalently connected to a C-terminal end of one of the two antibody heavy chain sequences, and the other of the at least two first antigen binding sites is covalently connected to a C-terminal end of the other of the two antibody heavy chain sequences.

49. The method of claim 48, wherein the binding sites are connected either without a peptide linker or with a short peptide linker having not more than 5 amino acids, or via a long peptide linker having at least 6, preferably up to 50 amino acids, wherein further preferably the peptide linker is a (GGGGS)ₙ linker, and n is larger or equal to two.

50. The method of claim 31, wherein the treatment comprises the sequential or concomitant administration of (i) a further binding molecule which is bispecific and which is capable of specifically binding to and activating a T cell, such as via binding to CD3 and/or a T cell receptor (TCR); or any other reagent capable of providing or enhancing signals for T cell activation such as (ii) genetically modified immune cells (heterologous or autologous T-cell) expressing an antigen receptor, such as a chimeric antigen receptor (CAR), which receptor is capable of specifically binding the antigenic target protein or (iii) vaccines providing antigenic structures from infectious agents or cancer cells (TAA or TSA) or (iv) reagents that block suppressive “second signals”, via checkpoint molecules such as PD1.

51. The method of claim 50, wherein the further binding molecule is at least bispecific and comprises at least one third antigen binding site and at least one fourth antigen binding site, wherein
(v) The least one third antigen binding site is capable of specifically binding to CD3 and/or a T cell receptor (TCR) (CD3/TCR); and
(vi) The at least one fourth antigen binding site is capable of specifically binding to an epitope of a further antigenic target protein expressed on or in a cell associated with the disease in the subject.

52. The method of claim 51, wherein the antigenic target protein and the further antigenic target protein are (i) identical or (ii) different but in close spatial proximity to each other, such as being expressed on the same cell associated with the disease or located in the same diseases tissue such as being expressed in the same tumor environment.

53. The method of claim 31, wherein the disease is a proliferative disease, preferably selected from a cancer disease, such as a cancer, for example lung cancer, breast cancer, colorectal cancer, gastric cancer, hepatocellular carcinoma, pancreatic cancer, ovarian cancer, melanoma, myeloma, kidney cancer, head and neck cancer, Hodgkin lymphoma, bladder cancer or prostate cancer, in particular one selected from the list consisting of: melanoma, lung cancer (such as non-small cell lung cancer), bladder cancer (such as urothelial carcinoma), kidney cancer (such as renal cell carcinoma), head and neck cancer (such as squamous cell cancer of the head and neck) and Hodgkin lymphoma. Preferably, the proliferative disease is melanoma, or lung cancer (such as non-small cell lung cancer), preferably a cancer positive for an expression of the target antigenic protein.

54. An isolated binding molecule, wherein the isolated binding molecule is the binding molecule as recited in claim 31.

55. An isolated nucleic acid encoding for an isolated binding molecule of claim 54.

56. A recombinant host cell comprising an isolated binding molecule of claim 54.

57. A pharmaceutical composition comprising an isolated binding molecule of claim 54, together with a pharmaceutically acceptable carrier and/or excipient.

58. The pharmaceutical composition of claim 57, further comprising an isolated further binding molecule:
(a) which is bispecific and which is capable of specifically binding to and activating a T cell, such as via binding to CD3 and/or a T cell receptor (TCR); or
(b) which is at least bispecific and comprises at least one third antigen binding site and at least one fourth antigen binding site, wherein
(i) the least one third antigen binding site is capable of specifically binding to CD3 and/or a T cell receptor (TCR) (CD3/TCR); and
the at least one fourth antigen binding site is capable of specifically binding to an epitope of a further antigenic target protein expressed on or in a cell associated with the disease in the subject.

59. A kit of packages or pharmaceutical compositions, the kit comprising in separate containers:
(i) an isolated binding molecule recited in claim 31, an isolated nucleic acid encoding the isolated binding molecule, and/or a recombinant host cell comprising such nucleic acid or isolated binding molecule; and
(ii) an isolated further binding molecule recited in claim 31, an isolated nucleic acid encoding the isolated further binding molecule, and/or a recombinant host cell comprising such nucleic acid or isolated further binding molecule.
